(19) United States
(12) Patent Application Publication
(10) Pub. No.: US 2020/0223889 A1
(43) Pub. Date: Jul. 16, 2020

(54) Bicyclic Compounds Capable of Binding to Melanocortin 4 Receptor

(71) Applicant: Novo Nordisk A/S, Bagsvaerd (DK)

(72) Inventors: Kilian Waldemar Conde Frieboes, Maaloev (DK); Christian Wenzel Tornoee, Lyngby (DK); Line Marie Nielsen, Frederiksberg (DK)

(21) Appl. No.: 16/492,838

(22) PCT Filed: Mar. 15, 2018
(86) PCT No.: PCT/EP2018/056482

§ 371 Date: Sep. 10, 2019

(30) Foreign Application Priority Data
Mar. 15, 2017 (EP) 17161142.9
Nov. 28, 2017 (EP) 17204100.6

(51) Int. Cl. C07K 7/64; A61P 3/04

(52) U.S. Cl. CPC C07K 7/64; A61K 38/00; A61P 3/04

(57) ABSTRACT

The present invention relates to novel peptide compounds which are effective as melanocortin 4 receptor agonists, to the use of the compounds in medicine, to methods of treatment comprising administration of the compounds to patients in need thereof, and to the use of the compounds for the manufacture of 5 medicaments. The compounds of the invention are of particular interest in relation to the treatment of obesity or overweight as well as a variety of diseases or conditions associated with obesity.

### INCORPORATION-BY-REFERENCE OF THE SEQUENCE LISTING

The Sequence Listing, entitled “Sequence Listing”, is 59.9 kilobytes, was created on 5 Mar. 2018 and is incorporated herein by reference.

### BACKGROUND

Obesity is a well-known risk factor for the development of common diseases such as atherosclerosis, hypertension, type 2 diabetes, dyslipidaemia, coronary heart disease, gallbladder disease, osteoarthritis, premature death, certain types of cancer and various other malignancies. It also causes considerable problems through reduced motility and decreased quality of life. In the industrialized Western world the prevalence of obesity has increased significantly in the past few decades. Only a few pharmacological treatments are available to date, SAXENDA (liraglutide), a GLP-1 receptor agonist from Novo Nordisk, BELVIQ (Lorcaserin), a 5-HT2C agonist from Arena, CONTRAVE (naltrexone/bupropion) a combination product of bupropion and naltraxone from Orexigen and orlistat from Roche and GlaxoSmithKline, works by reducing fat uptake from the gut. Because obesity represents a very high risk factor in serious and even fatal common diseases, its treatment should be a high public health priority and there is a need for pharmaceutical compounds useful in the treatment of obesity. Pro-opiomelanocortin (POMC) is the precursor of the melanocortin family of peptides, which include alpha-, beta- and gamma-melanocyte stimulating hormone (MSH) peptides and adrenocorticotropic hormone (ACTH), as well as other peptides such as beta-endorphin. POMC is expressed in neurons of the central and peripheral nervous system and in the pituitary. Several of the melanocortin peptides, including ACTH and alpha-MSH (a-MSH), have been shown to have appetite-suppressing activity when administered to rats by intracerebroventricular (icy) injection (Vergoni et al. (1990) Eur J Pharmacol 179(3):347-355).

Five melanocortin receptor subtypes have been identified: melanocortin 1, 2, 3, 4 and 5 receptor (from herein on also referred to as MC1R, MC2R, MC3R, MC4R and MC5R, respectively). MC1R, MC2R and MC5R are mainly expressed in peripheral tissues, whereas MC3R and MC4R are mainly centrally expressed. MC3R is also expressed in several peripheral tissues. In addition to being involved in energy homeostasis, MC3R have also been suggested to be involved in several inflammatory diseases. It has been suggested that MC5R is involved in exocrine secretion and in inflammation. MC4R have been shown to be involved in the regulation of body weight and feeding behaviour, as MC4R knock-out mice develop obesity (Huszar et al. (1997) Cell 88(1):131-141) and common variants in the MC4R locus have been found to be associated with fat mass, weight and risk of obesity (Loos et al. (2008) Nat Genet 40(6):768-775). Furthermore, studies with mice showed that overexpression in the mouse brain of the melanocortin receptor antagonists agouti protein and agouti-related protein (AGRP), led to the development of obesity (Barsh et al. (1999) Ann NY Acad Sci 885:143-152). Moreover, icy injection of a C-terminal fragment of AGRP increases feeding and antagonizes the inhibitory effect of a-MSH on food intake. MC4R agonists could serve as anorectic drugs and/or energy expenditure increasing drugs and be useful in the treatment of obesity or obesity-related diseases, as well as in the treatment of other diseases, disorders or conditions which may be ameliorated by activation of MC4R or in genetic disorders such as POMC deficiency (Kuhnen et al. (2016) N Engl J Med 375(3):240-246). Oppositely, MC4R antagonists may be useful in the treatment of cachexia or anorexia, of wasting in frail elderly patients, chronic pain, neuropathy and neurogenic inflammation.

The use of peptides as melanocortin receptor modulators is disclosed in a number of patent documents, e.g. WO03/006620, U.S. Pat. No. 5,731,408, WO98/27113, and US2016022764 and in the literature, e.g. Odagami et al. (2006) Bioorg Med Chem Lett 16(14):3723-3726. Setmelanotide (RM493) is a MC4R agonist which is currently being tested in clinical trial for use in the treatment of rare genetic disorders of obesity (Kuhnen et al. (2016) N Engl J Med 375(3):240-246. It remains a challenge to provide melanocortin receptor agonists which are highly potent and have an appropriate selectivity towards MC4R as compared to other melanocortin receptor subtypes.

### SUMMARY

The present invention relates to novel compounds which are capable of acting as melanocortin 4 receptor (MC4R) agonists.

In one aspect, the present invention relates to a bicyclic compound having the general Formula I:

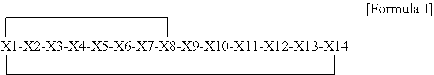

wherein

X1 and X8 are joined and X1 and X14 are joined;

X1 is Cys, HCys or Pen;

X2 is Ala, Pro, Hyp, THAZ, Aib, D-Ala, βAla, Sar, D-Pro, Val, D-Val, ACBC, GABA, ACP, Glu, Lys, D-Lys, Arg, AZE, PIP, OXA, Gly or absent;

X3 is His, Pro or Hyp;

X4 is D-Phe or Phe;

X5 is Arg, Lys, HArg, His, Dab, Dap, Cit, Orn, Arg(NO₂), N-MeArg, 4cis-GuaPro, 4trans-GuaPro, AGP, AcLys, Gln or Lys(Me)₂;

X6 is Trp, 2-Nal, 4-CN-Phe, 3,4-DiCl-Phe, 3,4-DiMeO-Phe or 1-Nal;

X7 is Ala, Glu, Gly, D-Ala, Arg or absent;

X8 is Cys, HCys or Pen;

X9 is Ala, Pro, Hyp, THAZ, Aib, D-Ala, βAla, Sar, D-Pro, Val, D-Val, ACBC, GABA, Arg, ACP, Glu, Lys, AZE, PIP, Orn, Gly, D-Lys or absent;

X10 is His, Pro, Hyp, Phe, Glu, Lys, D-Lys, Tyr, Ala, D-Ala, Asp, Arg or Orn;

X11 is D-Phe, Phe, Glu, Lys, Lys(CH₂COOH)₂, D-Lys, D-Ala, Ala, Arg, D-Arg or Asp;

X12 is Arg, Lys, HArg, His, Dab, Dap, Cit, Ala, Orn, Arg(NO₂), N-MeArg, 4cis-GuaPro, 4trans-GuaPro, AGP, Glu, Ala, Orn, D-Lys, Lys(Me)₂, Asn or absent;

X13 is Trp, Arg, 2-Nal, 4-CN-Phe, 3,4-DiCl-Phe, 3,4-DiMeO-Phe, 1-Nal, Ala, Phe or absent; X14 is Ala, Glu, Gly, D-Ala, Arg, Phe or absent;

including all enantiomers and diastereomers thereof, or a pharmaceutically acceptable salt of any of the foregoing.

In one aspect X1 and X8 are joined by a covalent bond such as a disulphide bond (Sₓ₁—Sₓ₈) or by a methylene bridge (Sₓ₁—CH₂—Sₓ₈) wherein Sₓ₁ and Sₓ₈ represent the sulfur atom in the X1 and X8 amino acid residue side chains, respectively. In one aspect X1 and X14 are joined by a covalent bond such as an amide bond between the alpha amine of X1 and the alpha carboxylic group of X14.

The invention further relates to the manufacture of compounds of the invention, use of compounds of the invention in medicine, such as (but not limited to) the treatment of obesity or overweight, to pharmaceutical compositions comprising compounds of the invention as well as an injection device with content thereof, and to the use of compounds of the invention for the manufacture of medicaments.

### BRIEF DESCRIPTION OF DRAWINGS

FIG. 1 shows mass spectrometry data for the compounds disclosed herein.

FIG. 2 shows daily and cumulative food intake, and relative body weight in diet-induced obese mice treated once daily with vehicle or compound as indicated. Data are mean±SEM.

### BRIEF DESCRIPTION OF THE SEQUENCES

SEQ ID NOs:1-121 represent the sequences of chem. 1-121.

### DESCRIPTION

Compounds of the present invention are capable of binding to melanocortin 4 receptor (MC4R) and can serve as MC4R agonists and are thus suited for the treatment of states and diseases which can be treated by stimulating MC4R activity. In particular, compounds of the present invention are believed to be suited for the treatment of diseases or states via activation of MC4R.

In one aspect the compounds of the invention are suitable for the treatment of obesity or overweight.

The invention further relates to the manufacture of compounds of the invention, use of compounds of the invention in medicine, to pharmaceutical compositions comprising compounds of the invention as well as an injection device with content thereof, and to the use of compounds of the invention for the manufacture of medicaments.

In one aspect of the present invention, the compound is a peptide.

In one such aspect the compound is a bicyclic peptide.

Bicyclic peptides as described herein can be peptides having two links between amino acid residues of the peptide which are not present in non-cyclic peptides. The bicyclic peptides may also or alternatively be described as peptide structures with two macrocyclic rings, so-called loops. In one aspect, bicyclic peptides of the invention comprise a bridging link between the side chains of two amino acids of the peptide and a link between the amino terminus and the carboxyl terminus of the peptide if represented as a linear peptide, i.e. such bicyclic peptide does not contain a free amino terminus or a free carboxyl terminus. In one aspect, the bridging link between the side chains of two amino acids is formed as a disulfide bond (for example between two cysteine residues) or alternatively as a methylene bridge (for example between two penicillamine residues).

In one aspect the bicyclic peptides of the invention are highly selective for MC4R.

In one aspect the bicyclic compounds of the invention are described according to Formula I:

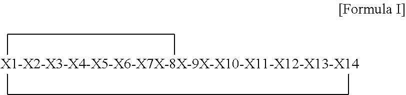

wherein

X1 and X8 are joined and X1 and X14 are joined;

X1 is Cys, HCys or Pen;

X2 is Ala, Pro, Hyp, THAZ, Aib, D-Ala, βAla, Sar, D-Pro, Val, D-Val, ACBC, GABA, ACP, Glu, Lys, D-Lys, Arg, AZE, PIP, OXA, Gly or absent;

X3 is His, Pro or Hyp;

X4 is D-Phe or Phe;

X5 is Arg, Lys, HArg, His, Dab, Dap, Cit, Orn, Arg(NO₂), N-MeArg, 4cis-GuaPro, 4trans-GuaPro, AGP, AcLys, Gln or Lys(Me)₂;

X6 is Trp, 2-Nal, 4-CN-Phe, 3,4-DiCl-Phe, 3,4-DiMeO-Phe, or 1-Nal;

X7 is Ala, Glu, Gly, D-Ala, Arg or absent;

X8 is Cys, HCys or Pen;

X9 is Ala, Pro, Hyp, THAZ, Aib, D-Ala, βAla, Sar, D-Pro, Val, D-Val, ACBC, GABA, Arg, ACP, Glu, Lys, AZE, PIP, Orn, Gly, D-Lys or absent;

X10 is His, Pro, Hyp, Phe, Glu, Lys, D-Lys, Tyr, Ala, D-Ala, Asp, Arg or Orn;

X11 is D-Phe, Phe, Glu, Lys, Lys(CH₂COOH)₂, D-Lys, D-Ala, Ala, Arg, D-Arg or Asp;

X12 is Arg, Lys, HArg, His, Dab, Dap, Cit, Ala, Orn, Arg(NO₂), N-MeArg, 4cis-GuaPro, 4trans-GuaPro, AGP, Glu, Ala, Orn, D-Lys, Lys(Me)₂, Asn or absent;

X13 is Trp, Arg, 2-Nal, 4-CN-Phe, 3,4-DiCl-Phe, 3,4-DiMeO-Phe, 1-Nal, Ala, Phe or absent;

X14 is Ala, Glu, Gly, D-Ala, Arg, Phe or absent;

including all enantiomers and diastereomers thereof, or a pharmaceutically acceptable salt of any of the foregoing.

In one embodiment X1 and X8 are joined by B1 as shown in Formula II

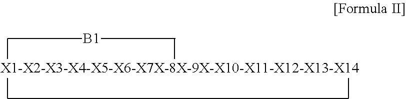

wherein X1 to X14 are as defined for Formula I,

wherein B1 is selected from a group consisting of:

*—CH₂—S—S—CH₂—**;

*—CH₂—S—S—(CH₂)₂—**;

*—(CH₂)₂—S—S—CH₂—**;

*—(CH₂)₂—S—S—(CH₂)₂—**;

*—CH₂—S—S—C(CH₃)₂—**;

*—C(CH₃)₂—S—S—CH₂—**;

*—C(CH₃)₂—S—S—C(CH₃)₂—**;

*—(CH₂)₂—S—S—C(CH₃)₂—**;

*—C(CH₃)₂—S—S—(CH₂)₂—**;

*—CH₂—S—(CH₂)_{z}—S—CH₂—**;

*—CH₂—S—(CH₂)_{z}—S—(CH₂)₂—**;

*—(CH₂)₂—S—(CH₂)_{z}—S—CH₂—**;

*—(CH₂)₂—S—(CH₂)_{z}—S—(CH₂)₂—**;

*—CH₂—S—(CH₂)_{z}—S—C(CH₃)₂—**;

*—C(CH₃)₂—S—(CH₂)_{z}—S—CH₂—**;

*—C(CH₃)₂—S—(CH₂)_{z}—S—C(CH₃)₂—**;

*—(CH₂)₂—S—(CH₂)_{z}—S—C(CH₃)₂—**; and

*—C(CH₃)₂—S—(CH₂)_{z}—S—(CH₂)₂—**

wherein

z is 1, 2, 3, 4 or 5; and wherein * and ** designates the backbone amino acid alpha-carbon atoms of X1 and X8, respectively.

In a preferred embodiment z is 1.

In one embodiment X1 and X8 are covalently joined by B1, wherein B1 comprises a disulphide bond (—S—S—) or a methylene bridge (—S—CH₂—S—).

X1 and X14 are joined by a covalent bond such as an amide bond between the alpha amine of X1 and the alpha carboxylic group of X14.

Certain of the residues designated X1 to X14 may be absent. In one such embodiment for example X2 and X7 can be absent and the bicyclic compound would consequently comprise a combination of the following amino acid residues:

X1 is Cys, HCys or Pen;

X3 is His, Pro or Hyp;

X4 is D-Phe or Phe;

X5 is Arg, Lys, HArg, His, Dab, Dap, Cit, Orn, Arg(NO₂), N-MeArg, 4cis-GuaPro, 4trans-GuaPro, AGP, AcLys, Gln or Lys(Me)₂;

X6 is Trp, 2-Nal, 4-CN-Phe, 3,4-DiCl-Phe, 3,4-DiMeO-Phe or 1-Nal;

X8 is Cys, HCys or Pen;

X9 is Ala, Pro, Hyp, THAZ, Aib, D-Ala, βAla, Sar, D-Pro, Val, D-Val, ACBC, GABA, Arg, ACP, Glu, Lys, AZE, PIP, Orn, Gly, D-Lys or absent;

X10 is His, Pro, Hyp, Phe, Glu, Lys, D-Lys, Tyr, Ala, D-Ala, Asp, Arg or Orn;

X11 is D-Phe, Phe, Glu, Lys, Lys(CH₂COOH)₂, D-Lys, D-Ala, Arg, D-Arg, Asp or Ala;

X12 is Arg, Lys, HArg, His, Dab, Dap, Cit, Ala, Orn, Arg(NO₂), N-MeArg, 4cis-GuaPro, 4trans-GuaPro, AGP, Glu, Ala, Orn, D-Lys, Lys(Me)₂, Asn or absent;

X13 is Trp, Arg, 2-Nal, 4-CN-Phe, 3,4-DiCl-Phe, 3,4-DiMeO-Phe, 1-Nal, Ala, Phe or absent;

X14 is Ala, Glu, Gly, D-Ala, Arg, Phe or absent.

In another embodiment for example X7 and X9 can be absent and the bicyclic compound would comprise a combination of the following amino acid residues:

X1 is Cys, HCys or Pen;

X2 is Ala, Pro, Hyp, THAZ, Aib, D-Ala, βAla, Sar, D-Pro, Val, D-Val, ACBC, GABA, ACP, Glu, Lys, D-Lys, Arg, AZE, PIP, OXA, Gly or absent;

X3 is His, Pro or Hyp;

X4 is D-Phe or Phe;

X5 is Arg, Lys, HArg, His, Dab, Dap, Cit, Orn, Arg(NO₂), N-MeArg, 4cis-GuaPro, 4trans-GuaPro, AGP, AcLys, Gln or Lys(Me)₂;

X6 is Trp, 2-Nal, 4-CN-Phe, 3,4-DiCl-Phe, 3,4-DiMeO-Phe or 1-Nal;

X8 is Cys, HCys or Pen;

X10 is His, Pro, Hyp, Phe, Glu, Lys, D-Lys, Tyr, Ala, D-Ala, Asp, Arg or Orn;

X11 is D-Phe, Phe, Glu, Lys, Lys(CH₂COOH)₂, D-Lys, D-Ala, Arg, D-Arg, Asp or Ala;

X12 is Arg, Lys, HArg, His, Dab, Dap, Cit, Ala, Orn, Arg(NO₂), N-MeArg, 4cis-GuaPro, 4trans-GuaPro, AGP, Glu, Ala, Orn, D-Lys, Lys(Me)₂, Asn or absent;

X13 is Trp, Arg, 2-Nal, 4-CN-Phe, 3,4-DiCl-Phe, 3,4-DiMeO-Phe, 1-Nal, Ala, Phe or absent;

X14 is Ala, Glu, Gly, D-Ala, Arg, Phe or absent.

Generally, in one embodiment, when a residue in Formula I is absent it means that it is replaced by a bond. For example, if X2 is absent it is replaced by a bond connecting X1 and X3.

Also or alternatively, in other embodiments, if X14 is absent the second cyclic bond (the one shown in Formula I between X1 and X14) will be established between X1 and X13. Likewise, if X13 and X14 are both absent the second cyclic bond will be established between X1 and X12. And similarly, if X12, X13, and X14 are all absent, the second cyclic bond will be formed between X1 and X11.

In one aspect of the present invention, the compound is a selective agonist of MC4R. In this context, if a compound is significantly more potent as a MC4R agonist than as a MC1R, MC3R and/or MC5R agonist, it is deemed to be a selective MC4R agonist. Selectively for MC4R over MC1R is desirable since MC1R is known to be associated with skin pigmentation.

The binding affinity of a compound of the present invention with respect to MC1R, MC3R, MC5R and MC4R may be determined by comparing the Ki value determined in a binding assay as described in Example 2 herein.

Compounds of the invention that act as MC4R agonists could have a positive effect on insulin sensitivity, on drug abuse (by modulating the reward system) and/or on hemorrhagic shock. In particular compounds of the invention may be used in the prevention and treatment of overweight or obesity. Furthermore, MC4R agonists have antipyretic effects, and have been suggested to be involved in peripheral nerve regeneration. MC4R agonists are also known to reduce stress response. In addition to treating drug abuse, treating or preventing hemorrhagic shock, and reducing stress response, compounds of the invention may also be of value in treating alcohol abuse, treating stroke, treating ischemia and protecting against neuronal damage.

As already indicated, in all of the therapeutic methods or indications disclosed above, the compound of the present invention may be administered alone. However, it may also be administered in combination with one or more additional therapeutically active agents, substances or compounds, either sequentially or concomitantly.

Compounds of the invention comprise compounds that are believed to be well-suited for administration twice daily, once daily, one every second day, twice-weekly or once-weekly administration by a suitable route of administration, such as one of the routes disclosed herein.

A typical dosage of a compound of the invention when employed in a method according to the present invention is in the range of from about 0.001 to about 100 mg/kg body weight per day, preferably from about 0.01 to about 10 mg/kg body weight, more preferably from about 0.01 to about 5 mg/kg body weight per day, e.g. from about 0.05 to about 10 mg/kg body weight per day or from about 0.03 to about 5 mg/kg body weight per day administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art. Compounds of the invention may conveniently be formulated in unit dosage form using techniques well known to those skilled in the art.

A typical unit dosage form intended for oral or parenteral administration may suitably contain from about 0.05 to about 1000 mg, preferably from about 0.05 to about 500 mg, such as from about 0.1 to about 200 mg of a compound of the invention.

As described above, compounds of the present invention may be administered or applied in combination with one or more additional therapeutically active compounds or substances, and suitable additional compounds or substances may be selected, for example, from anti-diabetic agents, anti-hyperlipidemic agents, anti-obesity agents, anti-hypertensive agents and agents for the treatment of complications resulting from, or associated with, diabetes.

Suitable anti-diabetic agents include insulin, insulin derivatives or analogues, GLP-1 (glucagon like peptide-1) derivatives or analogues such as those disclosed in WO98/08871 (Novo Nordisk A/S), which is incorporated herein by reference, or other GLP-1 analogues such as semaglutide (Novo Nordisk), exenatide (Byetta, Eli Lilly/Amylin; AVE0010, Sanofi-Aventis), taspoglutide (Roche), albiglutide (Syncria, GlaxoSmithKline), amylin, amylin analogues (e.g. Symlin™/Pramlintide) as well as orally active hypoglycemic agents.

Suitable orally active hypoglycemic agents include: metformin, imidazolines; sulfonylureas; biguanides; meglitinides; oxadiazolidinediones; thiazolidinediones; insulin sensitizers; α-glucosidase inhibitors; agents acting on the ATP-dependent potassium channel of the pancreatic β-cells, e.g. potassium channel openers such as those disclosed in WO97/26265, WO99/03861 and WO00/37474 (Novo Nordisk A/S) which are incorporated herein by reference; potassium channel openers such as ormitiglinide; potassium channel blockers such as nateglinide or BTS-67582; glucagon receptor antagonists such as those disclosed in WO99/01423 and WO00/39088 (Novo Nordisk A/S and Agouron Pharmaceuticals, Inc.), all of which are incorporated herein by reference; GLP-1 receptor agonists such as those disclosed in WO00/42026 (Novo Nordisk A/S and Agouron Pharmaceuticals, Inc.), which are incorporated herein by reference; amylin analogues (agonists on the amylin receptor); DPP-IV (dipeptidyl peptidase-IV) inhibitors; PTPase (protein tyrosine phosphatase) inhibitors; glucokinase activators, such as those described in WO02/08209 (Hoffmann La Roche); inhibitors of hepatic enzymes involved in stimulation of gluconeogenesis and/or glycogenolysis; glucose uptake modulators; GSK-3 (glycogen synthase kinase-3) inhibitors; compounds modifying lipid metabolism, such as anti-hyperlipidemic agents and anti-lipidemic agents; compounds lowering food intake; as well as PPAR (peroxisome proliferator-activated receptor) agonists and RXR (retinoid X receptor) agonists such as ALRT-268, LG-1268 or LG-1069. Further examples of suitable additional therapeutically active substances include thiazolidinedione insulin sensitizers, e.g. troglitazone, ciglitazone, pioglitazone, rosiglitazone, isaglitazone, darglitazone, englitazone, CS-011/CI-287 or T 174, or the compounds disclosed in WO97/41097 (DRF-2344), WO97/41119, WO97/41120, WO00/41121 and WO98/45292 (Dr. Reddy's Research Foundation), the contents of all of which are incorporated herein by reference. Additional examples of suitable additional therapeutically active substances include insulin sensitizers, e.g. GI 262570, YM-440, MCC-555, JTT-501, AR-H039242, KRP-297, GW-409544, CRE-16336, AR-H049020, LY510929, MBX-102, CLX-0940, GW-501516 and the compounds disclosed in WO99/19313 (NN622/DRF-2725), WO00/50414, WO00/63191, WO00/63192 and WO00/63193 (Dr. Reddy's Research Foundation), and in WO00/23425, WO 00/23415, WO00/23451, WO00/23445, WO00/23417, WO00/23416, WO 00/63153, WO00/63196, WO00/63209, WO00/63190 and WO 00/63189 (Novo Nordisk A/S), the contents of all of which are incorporated herein by reference. Still further examples of suitable additional therapeutically active substances include: α-glucosidase inhibitors, e.g. voglibose, emiglitate, miglitol or acarbose; glycogen phosphorylase inhibitors, e.g. the compounds described in WO97/09040 (Novo Nordisk A/S); glucokinase activators; agents acting on the ATP-dependent potassium channel of the pancreatic β-cells, e.g. tolbutamide, glibenclamide, glipizide, glicazide, BTS-67582 or repaglinide;

Other suitable additional therapeutically active substances include anti-hyperlipidemic agents and anti-lipidemic agents, e.g. cholestyramine, colestipol, clofibrate, gemfibrozil, lovastatin, pravastatin, simvastatin, probucol or dextrothyroxine.

Further agents which are suitable as additional therapeutically active substances include anti-obesity agents and appetite-regulating agents. Such substances may be selected from the group consisting of CART (cocaine amphetamine regulated transcript) agonists, NPY (neuropeptide Y receptor 1 and/or 5) antagonists, MC3 antagonists, orexin receptor antagonists, TNF (tumor necrosis factor) agonists, CRF (corticotropin releasing factor) agonists, CRF BP (corticotropin releasing factor binding protein) antagonists, urocortin agonists, neuromedin U analogues (agonists on the neuromedin U receptor subtypes 1 and 2), P3 adrenergic agonists such as CL-316243, AJ-9677, GW-0604, LY362884, LY377267 or AZ-40140, MCH (melanocyte-concentrating hormone) antagonists, CCK (cholecystokinin) agonists, serotonin reuptake inhibitors (e.g. fluoxetine, seroxat or citalopram), serotonin and norepinephrine reuptake inhibitors, 5HT (serotonin) agonists, 5HT6 agonists, 5HT2c agonists such as APD356 (U.S. Pat. No. 6,953,787), bombesin agonists, galanin antagonists, growth hormone, growth factors such as prolactin or placental lactogen, growth hormone releasing compounds, TRH (thyrotropin releasing hormone) agonists, UCP 2 or 3 (uncoupling protein 2 or 3) modulators, chemical uncouplers, leptin agonists, DA (dopamine) agonists (bromocriptin, doprexin), lipase/amylase inhibitors, TR 13 agonists, adrenergic CNS stimulating agents, AGRP (agouti-related protein) inhibitors, histamine H3 receptor antagonists such as those disclosed in WO00/42023, WO00/63208 and WO 00/64884, the contents of all of which are incorporated herein by reference, exendin-4 analogues, GLP-1 analogues, ciliary neurotrophic factor, amylin analogues, peptide YY₃₋₃₆ (PYY3-36) (Batterham et al. Nature 418, 650-654 (2002)), PYY3-36 analogues, NPY Y2 receptor agonists, NPY Y4 receptor agonists and substances acting as combined NPY Y2 and NPY Y4 agonists, FGF21 and analogues thereof, μ-opioid receptor antagonists, oxyntomodulin or analogues thereof.

Further suitable anti-obesity agents are bupropion (antidepressant), topiramate (anticonvulsant), ecopipam (dopamine D1/D5 antagonist) and naltrexone (opioid antagonist), and combinations thereof. Combinations of these anti-obesity agents would be e.g.: phentermine+topiramate, bupropion sustained release (SR)+naltrexone SR, zonisamide SR and bupropion SR. Among embodiments of suitable anti-obesity agents for use in a method of the invention as additional therapeutically active substances in combination with a compound of the invention are leptin and analogues or derivatives of leptin.

Additional embodiments of suitable anti-obesity agents are serotonin and norepinephrine reuptake inhibitors, e.g. sibutramine.

Other embodiments of suitable anti-obesity agents are lipase inhibitors, e.g. orlistat.

Still further embodiments of suitable anti-obesity agents are adrenergic CNS stimulating agents, e.g. dexamphetamine, amphetamine, phentermine, mazindol, phendimetrazine, diethylpropion, fenfluramine or dexfenfluramine. Other examples of suitable additional therapeutically active compounds include anti-hypertensive agents. Examples of anti-hypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. In certain embodiments of the uses and methods of the present invention, the compound of the present invention may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In the case, in particular, of administration of a compound of the invention, optionally in combination with one or more additional therapeutically active compounds or substances as disclosed above, for a purpose related to treatment or prevention of obesity or overweight, i.e. related to reduction or prevention of excess adiposity, it may be of relevance to employ such administration in combination with surgical intervention for the purpose of achieving weight loss or preventing weight gain, e.g. in combination with bariatric surgical intervention. The administration of a compound of the invention (optionally in combination with one or more additional therapeutically active compounds or substances as disclosed above) may take place for a period prior to carrying out the bariatric surgical intervention in question and/or for a period of time subsequent thereto. In many cases it may be preferable to begin administration of a compound of the invention after bariatric surgical intervention has taken place.

The compounds of the present invention can be a soluble MC4 receptor agonist, for example with solubility of at least 0.2 mmol/l, at least 0.5 mmol/l, at least 2 mmol/l, at least 4 mmol/l, at least 8 mmol/l, at least 10 mmol/l, or at least 15 mmol/l. determined at a suitable pH, such as pH 7.5, or a pH in the range of 6.0-8.0.

In the present context, if not stated otherwise, the terms “soluble”, “solubility”, “soluble in aqueous solution”, “aqueous solubility”, “water soluble”, “water-soluble”, “water solubility” and “water-solubility”, refer to the solubility of a compound in water or in an aqueous salt or aqueous buffer solution, for example a 10 mM phosphate solution, or in an aqueous solution containing other compounds, but no organic solvents.

In some embodiments the compounds of the present invention have high MC4R potency and higher MC4R selectivity compared to previously disclosed peptides in the art. In particular the compounds as disclosed herein may be highly selective for the MC4R as compared to the MC1R.

The term “obesity” implies an excess of adipose tissue. When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity. In this context, obesity is best viewed as any degree of excess adipose tissue that imparts a health risk. The distinction between normal and obese individuals can only be approximated, but the health risk imparted by obesity is probably a continuum with increasing adipose tissue. However, in the context of the present invention, individuals with a body mass index (BMI=body weight in kilograms divided by the square of the height in meters) between 25 and 30 are to be regarded as overweight and above 30 are to be regarded as obese.

In the present context, the term “agonist” is intended to indicate a compound (ligand) that activates the receptor type in question.

In the present context, the term “antagonist” is intended to indicate a compound (ligand) that blocks, neutralizes or counteracts the effect of an agonist.

In the present context, the term “pharmaceutically acceptable salt” is intended to indicate a salt which is not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acids, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. (1977) 66, 2, which is incorporated herein by reference. Examples of relevant metal salts include lithium, sodium, potassium and magnesium salts, and the like. Examples of alkylated ammonium salts include methylammonium, dimethylammonium, trimethylammonium, ethylammonium, hydroxyethylammonium, diethylammonium, butylammonium and tetramethylammonium salts, and the like.

As used herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

As used herein, the term “solvate” refers to a complex of defined stoichiometry formed between a solute (in casu, a compound according to the present invention) and a solvent. Solvents may include, by way of example, water, ethanol, or acetic acid.

The amino acids of the compounds of the invention include coded amino acids as well as non-coded amino acids. The coded amino acids are defined by IUPAC (first table in section 3AA-1): www.chem.qmul.ac.uk/iupac/AminoAcid/AA1n2.html#AA1, which gives structure, trivial name, systematic name, one- and three-letter symbols for the 20 coded amino acids. The one and three-letter symbols are also shown below:

| | |
|---|---|
| Ala (A) | Met (M) |
| Cys (C) | Asn (N) |
| Asp (D) | Pro (P) |
| Glu (E) | Gln (Q) |
| Phe (F) | Arg (R) |
| Gly (G) | Ser (S) |
| His (H) | Thr (T) |
| Ile (I) | Val (V) |
| Lys (K) | Trp (W) |
| Leu (L) | Tyr (Y) |

The following additional amino acid abbreviations when used in the present context have the following meanings:

| | | Chemical structure of | |
|---|---|---|---|
| | | the residue with | |
| Abbreviation | Name(s) of the amino acid | attachment points | Chem. no. |
| 1-Nal | 1-naphthylalanine | 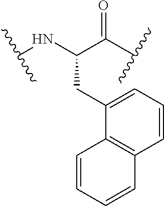 [SMILES: *N[C@@H](Cc1cccc2ccccc12)C(*)=O] | 122 |
| 2-Nal | 2-naphthylalanine | 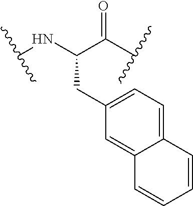 [SMILES: *N[C@@H](Cc1ccc2ccccc2c1)C(*)=O] | 123 |
| Dab | Diamino butyric acid | 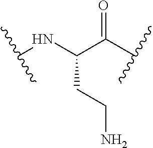 [SMILES: *N[C@@H](CCN)C(*)=O] | 124 |
| Dap | Diamino propionic acid | 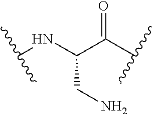 [SMILES: *N[C@@H](CN)C(*)=O] | 125 |
| Cit | Citrulline | 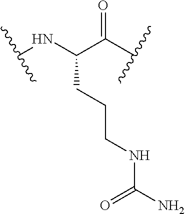 [SMILES: *N[C@@H](CCCNC(N)=O)C(*)=O] | 126 |
| 3,4-DiCl-Phe | 3,4-Dichloro-phenylalanine | 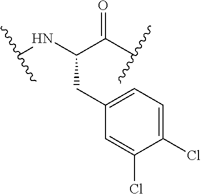 [SMILES: *N[C@@H](Cc1ccc(Cl)c(Cl)c1)C(*)=O] | 127 |
| Pen | Penicillamine | 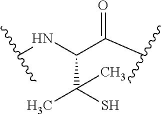 [SMILES: *N[C@H](C(*)=O)C(C)(C)S] | 128 |
| THAZ | Thiazolidine-4-carboxylic acid | 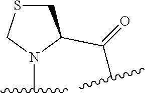 [SMILES: *C(=O)[C@@H]1CSCN1*] | 129 |
| OXA | Oxazolidine-4-carboxylic acid | 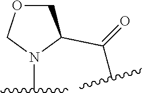 [SMILES: *C(=O)[C@@H]1COCN1*] | 130 |
| Hyp | Hydroxyproline | 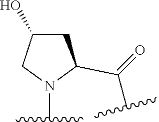 [SMILES: *C(=O)[C@@H]1C[C@@H](O)CN1*] | 131 |
| HCys | Homocysteine | 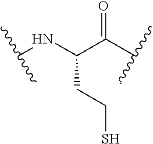 [SMILES: *N[C@@H](CCS)C(*)=O] | 132 |
| Orn | Ornithine | 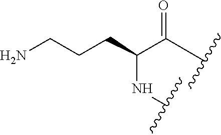 [SMILES: *N[C@@H](CCCN)C(*)=O] | 133 |
| Arg(NO₂) | Omega nitro arginine | 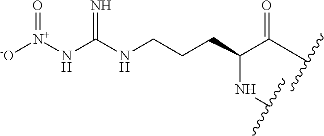 | 134 |
| N-MeArg | N-methyl arginine | 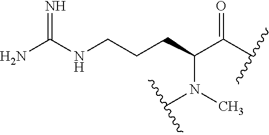 [SMILES: *C(=O)[C@H](CCCNC(=N)N)N(*)C] | 135 |
| 4cis-GuaPro | (2S,4S)-4- guanidinopyrrolidine-2- carboxylic acid | 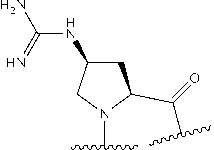 [SMILES: *C(=O)[C@@H]1C[C@H](NC(=N)N)CN1*] | 136 |
| 4trans- GuaPro | (2S,4R)-4- guanidinopyrrolidine-2- carboxylic acid | 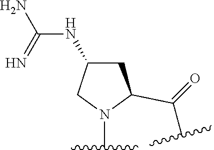 [SMILES: *C(=O)[C@@H]1C[C@@H](NC(=N)N)CN1*] | 137 |
| AcLys | N-acetyl-lysine | 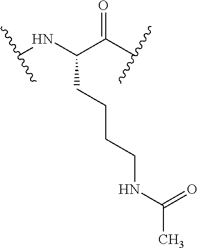 [SMILES: *N[C@@H](CCCCNC(C)=O)C(*)=O] | 138 |
| Lys(Me)₂ | N,N-dimethyl-lysine | 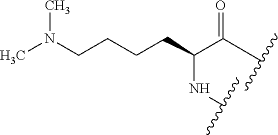 [SMILES: *N[C@@H](CCCCN(C)C)C(*)=O] | 139 |
| 4-CN-Phe | 4-cyano-phenylalanine | 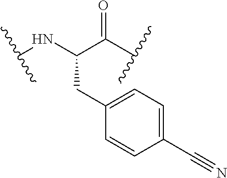 [SMILES: *N[C@@H](Cc1ccc(C#N)cc1)C(*)=O] | 140 |
| 3,4-DiMeO- Phe | 3,4-dimethoxy- phenylalanine | 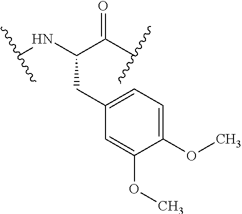 [SMILES: *N[C@@H](Cc1ccc(OC)c(OC)c1)C(*)=O] | 141 |
| Lys(CH₂COOH)₂ | N,N-bis(carboxymethyl)- lysine | 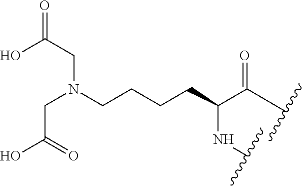 [SMILES: *N[C@@H](CCCCN(CC(=O)O)CC(=O)O)C(*)=O] | 142 |
| PIP | Piperidine-2-carboxylic acid | 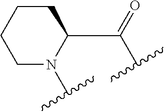 [SMILES: *C(=O)[C@@H]1CCCCN1*] | 143 |
| ACP | 4-aminopiperidine-4- carboxylic acid | 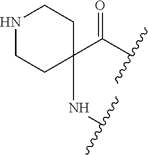 [SMILES: *NC1(C(*)=O)CCNCC1] | 144 |
| AZE | Azetidine-2-carboxylic acid | 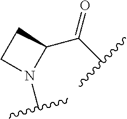 [SMILES: *C(=O)[C@@H]1CCN1*] | 145 |
| AGP | 2-amino-3-guanidino- propionic acid | 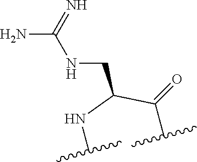 [SMILES: *N[C@@H](CNC(=N)N)C(*)=O] | 146 |
| ACBC | 1-amino cyclobutane carboxylic acid | 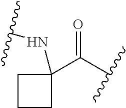 [SMILES: *NC1(C(*)=O)CCC1] | 147 |
| Aib | 2-amino isobutyric acid | 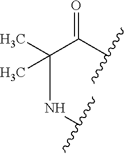 [SMILES: *NC(C)(C)C(*)=O] | 148 |
| Sar | Sarcosine | 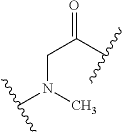 [SMILES: *C(=O)CN(*)C] | 149 |
| βAla | Beta alanine | 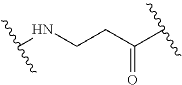 [SMILES: *NCCC(*)=O] | 150 |
| GABA | Gamma amino butyric acid | 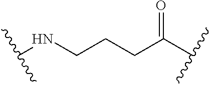 [SMILES: *NCCCC(*)=O] | 151 |
| HArg | Homo-arginine | 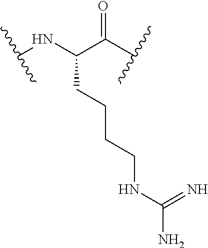 [SMILES: *N[C@@H](CCCCNC(=N)N)C(*)=O] | 152 |

In the present context, common rules for peptide nomenclature based on the three letter amino acid code apply, unless exceptions are specifically indicated. Briefly, the central portion of the amino acid structure is represented by the three letter code (e.g. Ala, Lys) and L-configuration is assumed, unless D-configuration is specifically indicated by “D-” followed by the three letter code (e.g. D-Ala, D-Lys). A substituent at the amino group replaces one hydrogen atom and its name is placed before the three letter code, whereas a C-terminal substituent replaces the carboxylic hydroxy group and its name appears after the three letter code. For example, “acetyl-Gly-Gly-NH₂” represents CH₃—C(═O)—NH—CH₂—C(═O)—NH—CH₂—C(═O)—NH₂. Unless indicated otherwise, amino acids with additional amino or carboxy groups in the side chains (such as Lys, Orn, Dap, Glu, Asp and others) are connected to their neighboring groups by amide bonds formed at the alpha amino group and the alpha carboxylic group.

The term “residue” with reference to an amino acid or amino acid derivative means a radical derived from the corresponding alpha-amino acid by eliminating the hydroxyl of the carboxy group and one hydrogen of the alpha amino group.

The term “joined” encompasses the linking of amino acid residues through covalent bonding, including disulfide bonding; methylene bonding (also referred to as methylene bridging), hydrogen bonding and electrostatic bonding.

When two amino acids are said to be covalently bonded, it is intended to indicate that functional groups in the side chains of the two respective amino acids have reacted to form a covalent bond.

Upon formation of a covalent bond between amino acid side chains, the compound may become cyclized. In case where the compound is already cyclic it may become bicyclic.

Such a (bi)cyclic polypeptide may be referred to by a structural formula or by using the short-hand notation “c[ ]” for cyclic compounds and c[c[ ]] for bicyclic compounds.

Bicyclic peptides may thus be referred to using the following short-hand notation c[c[Cys-His-D-Phe-Arg-Trp-Glu-Cys]-His-D-Phe-Arg-Trp-Glu] (Chem. 1), indicating—in the present example—that the Cys residues in position 1 and the Cys residue in position 7 are joined and that the Cys residue in position 1 and the Glu residue in position 12 are joined.

Unless otherwise indicated a Cys-Cys bond is a disulphide bond. For example, as an alternative to a regular disulphide bond the Cys residues may be joined by a methylene bridge.

The term “methylene bridge” as used herein refers to a methylene unit bound to the two sulphur atoms of two cysteine units thus bridging the two cysteines (R1-S—CH₂—S—R2, where R1 and R2 represent the remainder of the amino acid residue side chains).

As already mentioned, one aspect of the present invention provides a pharmaceutical composition comprising a compound as disclosed herein.

The term “composition” is used interchangeably with the term “formulation”. Appropriate embodiments of such formulations will often contain a compound as disclosed herein in a concentration of from 10⁻³ mg/ml to 50 mg/ml, such as, e.g., from 10⁻¹ mg/ml to 10 mg/ml.

The formulation may further comprise a buffer system, preservative(s), tonicity agent(s), chelating agent(s), stabilizer(s) and/or surfactant(s). In one embodiment of the invention the pharmaceutical formulation is an aqueous formulation, i.e. formulation comprising water, and the term “aqueous formulation” in the present context may normally be taken to indicate a formulation comprising at least 50% by weight (w/w) of water. Such a formulation is typically a solution or a suspension. An aqueous formulation of the invention in the form of an aqueous solution will normally comprise at least 50% (w/w) of water. Likewise, an aqueous formulation of the invention in the form of an aqueous suspension will normally comprise at least 50% (w/w) of water.

In another embodiment, a pharmaceutical composition of the invention may be a freeze-dried (i.e. lyophilized) formulation intended for reconstitution by the physician or the patient via addition of solvents and/or diluents prior to use.

In a further embodiment, a pharmaceutical composition of the invention may be a dried formulation (e.g. freeze-dried or spray-dried) ready for use without any prior dissolution.

In a further embodiment, the invention relates to a pharmaceutical composition comprising an aqueous solution of a compound of the present invention, and a buffer, wherein the compound of the invention is present in a concentration of 0.1-10 mg/ml or above, and wherein the formulation has a pH from about 2.0 to about 10.0.

The pH of a composition of the invention will typically be in the range of 2.0 to 10.0.

In one such embodiment, the pH of the composition has a value selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10.0.

In a further embodiment, the buffer in a buffered pharmaceutical composition of the invention may comprise one or more buffer substances selected from the group consisting of sodium acetate, sodium carbonate, citrates, glycylglycine, histidine, glycine, lysine, arginine, sodium dihydrogen phosphate, disodium hydrogen phosphate, sodium phosphate, tris(hydroxymethyl)aminomethane (TRIS), bicine, tricine, malic acid, succinates, maleic acid, fumaric acid, tartaric acid and aspartic acid. Each one of these specific buffers constitutes an alternative embodiment of the invention.

In another embodiment, a pharmaceutical composition of the invention may comprise a pharmaceutically acceptable preservative, e.g. one or more preservatives selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, thiomerosal, bronopol, benzoic acid, imidurea, chlorohexidine, sodium dehydroacetate, chlorocresol, ethyl p-hydroxybenzoate, benzethonium chloride and chlorphenesine (3-(4-chlorophenoxy)propane-1,2-diol). Each one of these specific preservatives constitutes an alternative embodiment of the invention. In a further embodiment of the invention the preservative is present in a concentration from 0.1 mg/ml to 20 mg/ml. In still further embodiments of such a pharmaceutical composition of the invention, the preservative is present in a concentration in the range of 0.1 mg/ml to 5 mg/ml, a concentration in the range of 5 mg/ml to 10 mg/ml, or a concentration in the range of 10 mg/ml to 20 mg/ml. The use of a preservative in pharmaceutical compositions is well known to the skilled person. For convenience, reference is made in this respect to Remington: The Science and Practice of Pharmacy, 20^{th} edition, 2000. In a further embodiment of the invention the formulation further comprises a tonicity-adjusting agent, i.e. a substance added for the purpose of adjusting the tonicity (osmotic pressure) of a liquid formulation (notably an aqueous formulation) or a reconstituted freeze-dried formulation of the invention to a desired level, normally such that the resulting, final liquid formulation is isotonic or substantially isotonic. Suitable tonicity-adjusting agents may be selected from the group consisting of salts (e.g. sodium chloride), sugars and sugar alcohols (e.g. mannitol), amino acids (e.g. glycine, histidine, arginine, lysine, isoleucine, aspartic acid, tryptophan or threonine), alditols (e.g. glycerol (glycerine), 1,2-propanediol (propyleneglycol), 1,3-propanediol or 1,3-butanediol), polyethyleneglycols (e.g. PEG 400) and mixtures thereof.

Any sugar, such as a mono-, di- or polysaccharide, or a water-soluble glucan, including for example fructose, glucose, mannose, sorbose, xylose, maltose, lactose, sucrose, trehalose, dextran, pullulan, dextrin, cyclodextrin, soluble starch, hydroxyethyl starch or carboxymethylcellulose-sodium, may be used; in one embodiment, sucrose may be employed. Sugar alcohols (polyols derived from mono-, di-, oligo- or polysaccharides) include, for example, mannitol, sorbitol, inositol, galactitol, dulcitol, xylitol, and arabitol. In one embodiment, the sugar alcohol employed is mannitol. Sugars or sugar alcohols mentioned above may be used individually or in combination. There is no fixed limit to the amount used, as long as the sugar or sugar alcohol is soluble in the liquid composition and does not adversely affect the stabilizing effects achieved using the methods of the invention. In one embodiment, the concentration of sugar or sugar alcohol is between about 1 mg/ml and about 150 mg/ml.

In further embodiments, the tonicity-adjusting agent is present in a concentration of from 1 mg/ml to 50 mg/ml, such as from 1 mg/ml to 7 mg/ml, from 8 mg/ml to 24 mg/ml, or from 25 mg/ml to 50 mg/ml. A pharmaceutical composition of the invention containing any of the tonicity-adjusting agents specifically mentioned above constitutes an embodiment of the invention. The use of a tonicity-adjusting agent in pharmaceutical compositions is well known to the skilled person. For convenience, reference is made to Remington: The Science and Practice of Pharmacy, 20^{th} edition, 2000.

In a still further embodiment of a pharmaceutical composition of the invention, the formulation further comprises a chelating agent. Suitable chelating agents may be selected, for example, from salts of ethylenediaminetetraacetic acid (EDTA), citric acid, and aspartic acid, and mixtures thereof. The concentration of chelating agent will suitably be in the range from 0.1 mg/ml to 5 mg/ml, such as from 0.1 mg/ml to 2 mg/ml or from 2 mg/ml to 5 mg/ml.

A pharmaceutical composition of the invention containing any of the chelating agents specifically mentioned above constitutes an embodiment of the invention. The use of a chelating agent in pharmaceutical compositions is well known to the skilled person. For convenience, reference is made to Remington: The Science and Practice of Pharmacy, 20^{th} edition, 2000.

In another embodiment of a pharmaceutical composition of the invention, the formulation further comprises a stabilizer. The use of a stabilizer in pharmaceutical compositions is well known to the skilled person. For convenience, reference is made to Remington: The Science and Practice of Pharmacy, 20^{th} edition, 2000.

More particularly, particularly useful compositions of the invention include stabilized liquid pharmaceutical compositions whose therapeutically active components include a compound that possibly exhibits aggregate formation during storage in liquid pharmaceutical formulations. By “aggregate formation” is meant the formation of oligomers, which may remain soluble, or large visible aggregates that precipitate from the solution, as the result of a physical interaction between the peptide molecules. The term “during storage” refers to the fact that a liquid pharmaceutical composition or formulation, once prepared, is not normally administered to a subject immediately. Rather, following preparation, it is packaged for storage, whether in a liquid form, in a frozen state, or in a dried form for later reconstitution into a liquid form or other form suitable for administration to a subject. By “dried form” is meant the product obtained when a liquid pharmaceutical composition or formulation is dried by freeze-drying (i.e., lyophilization; see, for example, Williams and Polli (1984) J. Parenteral Sci. Technol. 38: 48-59), by spray-drying [see, e.g., Masters (1991) in Spray-Drying Handbook (5th edn.; Longman Scientific and Technical, Essex, U.K.), pp. 491-676; Broadhead et al. (1992) Drug Devel. Ind. Pharm. 18: 1169-1206; and Mumenthaler et al. (1994) Pharm. Res. 11: 12-20], or by air-drying [see, e.g., Carpenter and Crowe (1988) Cryobiology 25: 459-470; and Roser (1991) Biopharm. 4: 47-53]. Aggregate formation by a compound of the present invention during storage of a liquid pharmaceutical composition can adversely affect biological activity of that peptide, resulting in loss of therapeutic efficacy of the pharmaceutical composition. Furthermore, aggregate formation may cause other problems, such as blockage of tubing, membranes or pumps when the peptide-containing pharmaceutical composition is administered using an infusion system.

A pharmaceutical composition of the invention may further comprise an amount of an amino acid base sufficient to decrease aggregate formation by a compound of the present invention during storage of the composition. By “amino acid base” is meant an amino acid, or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids for use in preparing a composition of the invention are those carrying a charged side chain, such as arginine, lysine, aspartic acid and glutamic acid. Any stereoisomer (i.e., L, D, or mixtures thereof) of a particular amino acid (e.g. methionine, histidine, arginine, lysine, isoleucine, aspartic acid, tryptophan or threonine, and mixtures thereof) or combinations of these stereoisomers, may be present in the pharmaceutical compositions of the invention so long as the particular amino acid is present either in its free base form or its salt form. In one embodiment, the L-stereoisomer of an amino acid is used. Compositions of the invention may also be formulated with analogues of these amino acids. By “amino acid analogue” is meant a derivative of a naturally occurring amino acid that brings about the desired effect of decreasing aggregate formation by a compound of the present invention during storage of liquid pharmaceutical compositions of the invention. Suitable arginine analogues include, for example, aminoguanidine, ornithine and N-monoethyl-L-arginine. Suitable methionine analogues include ethionine and buthionine, and suitable cysteine analogues include S-methyl-L-cysteine. As with the amino acids per se, amino acid analogues are incorporated into compositions of the invention in either their free base form or their salt form. In a further embodiment of the invention, the amino acids or amino acid analogues are incorporated in a concentration which is sufficient to prevent or delay aggregation of a compound of the present invention.

In a particular embodiment of the invention, methionine (or another sulfur-containing amino acid or amino acid analogue) may be incorporated in a composition of the invention to inhibit oxidation of methionine residues to methionine sulfoxide when a compound of the present invention acting as the therapeutic agent is a peptide comprising at least one methionine residue susceptible to such oxidation. The term “inhibit” in this context refers to minimization of accumulation of methionine-oxidized species over time. Inhibition of methionine oxidation results in increased retention of a compound of the present invention in its proper molecular form. Any stereoisomer of methionine (L or D) or combinations thereof can be used. The amount to be added should be an amount sufficient to inhibit oxidation of methionine residues such that the amount of methionine sulfoxide is acceptable to regulatory agencies. Typically, this means that no more than from about 10% to about 30% of forms of a compound of the present invention wherein methionine is sulfoxidated are present. In general, this can be achieved by incorporating methionine in the composition such that the ratio of added methionine to methionine residues ranges from about 1:1 to about 1000:1, such as from about 10:1 to about 100:1.

In a further embodiment of the invention the composition further comprises a stabilizer selected from high-molecular-weight polymers and low-molecular-weight compounds. Thus, for example, the stabilizer may be selected from substances such as polyethylene glycol (e.g. PEG 3350, Sigma-Aldrich), polyvinyl alcohol (PVA), polyvinylpyrrolidone, carboxy-/hydroxycellulose and derivatives thereof (e.g. HPC or HPMC), cyclodextrins, sulfur-containing substances such as monothioglycerol, thioglycolic acid and 2-methylthioethanol, and various salts (e.g. sodium chloride). A pharmaceutical composition of the invention containing any of the stabilizers specifically mentioned above constitutes an embodiment of the invention.

Pharmaceutical compositions of the present invention may also comprise additional stabilizing agents which further enhance stability of a therapeutically active compound therein. Stabilizing agents of particular interest in the context of the present invention include, but are not limited to: methionine and EDTA, which protect the peptide against methionine oxidation; and surfactants, notably nonionic surfactants which protect the polypeptide against aggregation or degradation associated with freeze-thawing or mechanical shearing.

Thus, in a further embodiment of the invention, the pharmaceutical composition comprises a surfactant, particularly a nonionic surfactant. Examples thereof include ethoxylated castor oil, polyglycolyzed glycerides, acetylated monoglycerides, sorbitan fatty acid esters, polyoxypropylene-polyoxyethylene block polymers (e.g. poloxamers such as PLURONIC F68, poloxamer 188 and 407, Triton X-100), polyoxyethylene sorbitan fatty acid esters, polyoxyethylene and polyethylene derivatives such as alkylated and alkoxylated derivatives (Tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g. phosphatidyl-serine, phosphatidyl-choline, phosphatidyl-ethanolamine, phosphatidyl-inositol, diphosphatidyl-glycerol and sphingomyelin), derivatives of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkyl ester and alkyl ether derivatives of lysophosphatidyl and phosphatidylcholines, e.g. lauroyl and myristoyl derivatives of lysophosphatidylcholine, dipalmitoylphosphatidylcholine, and modifications of the polar head group, i.e. cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (e.g. galactopyranoside), sphingoglycolipids (e.g. ceramides, gangliosides), dodecylphosphocholine, hen egg lysolecithin, fusidic acid derivatives (e.g. sodium tauro-dihydrofusidate, etc.), long-chain fatty acids (e.g. oleic acid or caprylic acid) and salts thereof, acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no. [577-11-7]), docusate calcium, CAS registry no. [128-49-4]), docusate potassium, CAS registry no. [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-aryl-sulfonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (e.g. Tetronic's), which are tetrafunctional block copolymers derived from sequential addition of propylene oxide and ethylene oxide to ethylenediamine. The surfactant may also be selected from imidazoline derivatives and mixtures thereof. A pharmaceutical composition of the invention containing any of the surfactants specifically mentioned above constitutes an embodiment of the invention.

The use of a surfactant in pharmaceutical compositions is well-known to the skilled person. For convenience, reference is made to Remington: The Science and Practice of Pharmacy, 20^{th} edition, 2000.

Additional ingredients may also be present in a pharmaceutical composition of the present invention. Such additional ingredients may include, for example, wetting agents, emulsifiers, antioxidants, bulking agents, metal ions, oleaginous vehicles, proteins (e.g. human serum albumin, gelatine or other proteins) and a zwitterionic species (e.g. an amino acid such as betaine, taurine, arginine, glycine, lysine or histidine). Such additional ingredients should, of course, not adversely affect the overall stability of the pharmaceutical formulation of the present invention.

Pharmaceutical compositions (formulations) containing a compound according to the present invention may be administered to a patient in need of such treatment at several sites, for example at topical sites (e.g. skin and mucosal sites), at sites which bypass absorption (e.g. via administration in an artery, in a vein or in the heart), and at sites which involve absorption (e.g. in the skin, under the skin, in a muscle or in the abdomen). Administration of pharmaceutical compositions (formulations) according to the invention to patients in need thereof may be via several routes of administration. These include, for example, lingual, sublingual, buccal, in the mouth, oral, in the stomach and intestine, nasal, pulmonary (for example through the bronchioles and alveoli or a combination thereof), epidermal, dermal, transdermal, vaginal, rectal, ocular (for example through the conjunctiva) and parenteral.

Parenteral administration may be performed by subcutaneous, intramuscular, intraperitoneal or intravenous injection by means of a syringe, for example a syringe in the form of a pen device. Alternatively, parenteral administration can be performed by means of an infusion pump.

A further option is administration of a composition of the invention which is a liquid (typically aqueous) solution or suspension in the form of a nasal or pulmonary spray. As a still further option, a pharmaceutical composition of the invention can be adapted to transdermal administration (e.g. by needle-free injection or via a patch, such as an iontophoretic patch) or transmucosal (e.g. buccal) administration.

Compositions of the present invention may be administered in various dosage forms, for example in the form of solutions, suspensions, emulsions, microemulsions, multiple emulsion, foams, salves, pastes, plasters, ointments, tablets, coated tablets, rinses, capsules (e.g. hard gelatine capsules or soft gelatine capsules), suppositories, rectal capsules, drops, gels, sprays, powder, aerosols, inhalants, eye drops, ophthalmic ointments, ophthalmic rinses, vaginal pessaries, vaginal rings, vaginal ointments, injection solutions, in situ-transforming solutions (for example in situ gelling, in situ setting, in situ precipitating or in situ crystallizing), infusion solutions or implants. Compositions of the invention may further be compounded in, or bound to, e,g. via covalent, hydrophobic or electrostatic interactions, a drug carrier, drug delivery system or advanced drug delivery system in order to further enhance the stability of the compound of the present invention, increase bioavailability, increase solubility, decrease adverse effects, achieve chronotherapy well known to those skilled in the art, and increase patient compliance, or any combination thereof. Examples of carriers, drug delivery systems and advanced drug delivery systems include, but are not limited to: polymers, for example cellulose and derivatives; polysaccharides, for example dextran and derivatives, starch and derivatives; poly(vinyl alcohol); acrylate and methacrylate polymers; polylactic and polyglycolic acid and block co-polymers thereof; polyethylene glycols; carrier proteins, for example albumin; gels, for example thermogelling systems, such as block co-polymeric systems well known to those skilled in the art; micelles; liposomes; microspheres; nanoparticulates; liquid crystals and dispersions thereof; L2 phase and dispersions thereof well known to those skilled in the art of phase behavior in lipid-water systems; polymeric micelles; multiple emulsions (self-emulsifying, self-microemulsifying); cyclodextrins and derivatives thereof; and dendrimers. Compositions of the present invention are useful in the formulation of solids, semisolids, powders and solutions for pulmonary administration of a compound of the present invention, using, for example, a metered dose inhaler, dry powder inhaler or a nebulizer, all of which are devices well known to those skilled in the art.

The term “stabilized formulation” refers to a formulation with increased physical stability, increased chemical stability or increased physical and chemical stability. The term “physical stability” in the context of a formulation containing a compound of the present invention refers to the tendency of the compound to form biologically inactive and/or insoluble aggregates as a result of exposure to thermo-mechanical stresses and/or interaction with interfaces and surfaces that are destabilizing, such as hydrophobic surfaces and interfaces. Physical stability of aqueous peptide/protein formulations is evaluated by means of visual inspection and/or turbidity measurements after exposing the formulation, filled in suitable containers (e.g. cartridges or vials), to mechanical/physical stress (e.g. agitation) at different temperatures for various time periods. Visual inspection of formulations is performed in a sharp focused light with a dark background. The turbidity of a formulation is characterized by a visual score ranking the degree of turbidity, for instance on a scale from 0 to 3 (in that a formulation showing no turbidity corresponds to a visual score 0, whilst a formulation showing visual turbidity in daylight corresponds to visual score 3). A formulation is normally classified physically unstable with respect to aggregation when it shows visual turbidity in daylight. Alternatively, the turbidity of a formulation can be evaluated by simple turbidity measurements well-known to the skilled person.

The term “chemical stability” of a pharmaceutical formulation as used herein refers to chemical covalent changes in compound structure leading to formation of chemical degradation products with potentially lower biological potency and/or potentially increased immunogenicity compared to the original molecule. Various chemical degradation products can be formed depending on the type and nature of the starting molecule and the environment to which it is exposed. Elimination of chemical degradation can most probably not be completely avoided and gradually increasing amounts of chemical degradation products may often be seen during storage and use of compound formulations, as is well known to the person skilled in the art. A commonly encountered degradation process is deamidation, a process in which the side-chain amide group in glutaminyl or asparaginyl residues is hydrolysed to form a free carboxylic acid. Other degradation pathways involve formation of higher molecular weight transformation products wherein two or more molecules of the starting substance are covalently bound to each other through transamidation and/or disulfide interactions, leading to formation of covalently bound dimer, oligomer or polymer degradation products (see, e.g., Stability of Protein Pharmaceuticals, Ahern. T. J. & Manning M. C., Plenum Press, New York 1992). Oxidation (of for instance methionine residues) may be mentioned as another variant of chemical degradation. The chemical stability of a formulation may be evaluated by measuring the amounts of chemical degradation products at various time-points after exposure to different environmental conditions (in that the formation of degradation products can often be accelerated by, e.g., increasing temperature). The amount of each individual degradation product is often determined by separation of the degradation products depending on molecule size and/or charge using various chromatographic techniques (e.g. SEC-HPLC and/or RP-HPLC).

Hence, as outlined above, a “stabilized formulation” refers to a formulation with increased physical stability, increased chemical stability, or increased physical and chemical stability. In general, a pharmaceutical composition must be stable during use and storage (in compliance with recommended use and storage conditions) until the expiry date is reached.

A pharmaceutical composition of the invention should preferably be stable for more than 2 weeks of usage and for more than 2 years of storage, more preferably for more than 4 weeks of usage and for more than 2 years of storage, desirably for more than 4 weeks of usage and for more than 3 years of storage, and most preferably for more than 6 weeks of usage and for more than 3 years of storage.

In one embodiment the present invention relates to an injection device with content thereof. In some embodiments the pharmaceutical composition of the invention is intended for use and/or contained in an injection device. In some embodiments, the injection device is a disposable, pre-filled, multi-dose pen of the FlexTouch® type (supplier Novo Nordisk A/S, Denmark). In some embodiments the injection device is a single shot device.

In some embodiments the injection device is a fixed dose device, such as one configured to deliver multiple predetermined doses of drug, sometimes referred to as a multiple fixed dose device or a fixed dose, multi-shot device.

The term “EC₅₀” as used herein refers to the molar concentration of an agonist, which produces 50% of the maximum possible response for that agonist. By way of example, a test compound which, at a concentration of 72 nM, produces 50% of the maximum possible response for that compound as determined in a cAMP assay in an MC4R cell expression system has an EC₅₀ of 72 nM. Unless otherwise specified, the molar concentration associated with an EC_{K} determination is in nanomoles per liter (nM).

The term “Kᵢ (nM)” as used herein refers to the equilibrium inhibitor dissociation constant representing the molar concentration of a competing compound that binds to half the binding sites of a receptor at equilibrium in the absence of radioligand or other competitors. Generally, the numeric value of the Ki is inversely correlated to the affinity of the compound for the receptor, such that if the Ki is low, the affinity is high. Ki may be determined using the equation (Math. 1) of Cheng and Prusoff (Cheng Y., Prusoff W. H., Biochem. Pharmacol. 22: 3099-3108, 1973):

$$Ki = \frac{{EC}_{50}}{1 + \frac{[ ligand ]}{{K}_{D}}} \qquad [ Math . 1 ]$$

where “ligand” is the concentration of radioligand and K_{D} is an inverse measure of receptor affinity for the radioligand which produces 50% receptor occupancy by the radioligand. Unless otherwise specified, the molar concentration associated with a Ki determination is in nM. Ki may be expressed in terms of specific receptors (e.g., MC1R, MC3R, MC4R or MC5R) and specific ligands (e.g., a-MSH or the compounds as disclosed herein).

The present invention encompasses variants of the bicyclic compounds of the invention which may comprise 1, 2 or, 3 amino acid substitutions and/or deletions and/or insertions in the specific sequences disclosed herein. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Substitutions may be, but are not limited to, conservative substitutions. Preferred variants include those in which instead of the amino acid which appears in the sequence comprises a structural analog of the amino acid.

All references, including publications, patent applications and patents, cited herein are hereby incorporated by reference in their entirety and to the same extent as if each reference were individually and specifically indicated to be incorporated by reference and were set forth in its entirety herein (to the maximum extent permitted by law).

The citation and incorporation of patent documents herein is done for convenience only, and does not reflect any view of the validity, patentability and/or enforceability of such patent documents.

Headings and sub-headings are used herein for convenience only, and should not be construed as limiting the invention in any way.

Unless otherwise indicated the term “about” means+/−10%.

The use of any and all examples, or exemplary language (including “for instance”, “for example”, “e.g.” and “such as”) in the present specification is intended merely to better illuminate the invention, and does not pose a limitation on the scope of the invention unless otherwise indicated. No language in the specification should be construed as indicating any non-claimed element as being essential to the practice of the invention.

The present invention includes all modifications and equivalents of the subject matter recited in the claims appended hereto, as permitted by applicable law.

### EMBODIMENTS

The invention is further described by the following non-limiting embodiments:
1. A bicyclic MC4R agonist compound comprising a sequence represented by the following amino acid residues Cys-X-His-D-Phe-His-Trp-Cys, wherein X is Pro or THAZ.
2. A bicyclic compound according to Formula I:

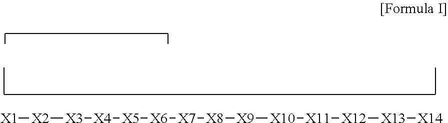

wherein
X1 and X8 are joined and X1 and X14 are joined;
X1 is Cys, HCys or Pen;
X2 is Ala, Pro, Hyp, THAZ, Aib, D-Ala, βAla, Sar, D-Pro, Val, D-Val, ACBC, GABA, ACP, Glu, Lys, D-Lys, Arg, AZE, PIP, OXA, Gly or absent;
X3 is His, Pro or Hyp;
X4 is D-Phe or Phe;
X5 is Arg, Lys, HArg, His, Dab, Dap, Cit, Orn, Arg(NO₂), N-MeArg, 4cis-GuaPro, 4trans-GuaPro, AGP, AcLys, Gln or Lys(Me)₂;
X6 is Trp, 2-Nal, 4-CN-Phe, 3,4-DiCl-Phe, 3,4-DiMeO-Phe or 1-Nal;
X7 is Ala, Glu, Gly, D-Ala, Arg or absent;
X8 is Cys, HCys or Pen;
X9 is Ala, Pro, Hyp, THAZ, Aib, D-Ala, βAla, Sar, D-Pro, Val, D-Val, ACBC, GABA, Arg, ACP, Glu, Lys, AZE, PIP, Orn, Gly, D-Lys or absent;
X10 is His, Pro, Hyp, Phe, Glu, Lys, D-Lys, Tyr, Ala, D-Ala, Asp, Arg or Orn;
X11 is D-Phe, Phe, Glu, Lys, Lys(CH₂COOH)₂, D-Lys, D-Ala, Ala, Arg or Asp;
X12 is Arg, Lys, HArg, His, Dab, Dap, Cit, Ala, Orn, Arg(NO₂), N-MeArg, 4cis-GuaPro, 4trans-GuaPro, AGP, Glu, Ala, Orn, D-Lys, Lys(Me)₂, Asn or absent;
X13 is Trp, Arg, 2-Nal, 4-CN-Phe, 3,4-DiCl-Phe, 3,4-DiMeO-Phe, 1-Nal, Ala, Phe or absent;
X14 is Ala, Glu, Gly, D-Ala, Arg, Phe or absent;
including all enantiomers and diastereomers thereof, or a pharmaceutically acceptable salt of any of the foregoing.
3. The bicyclic compound according to embodiment 1 or 2 wherein said compound comprises a disulphide bond or a methylene bridge.
4. The compound according to embodiment 2 or 3, wherein X1 and X8 are amino acid residues comprising a sulfur atom.
5. The compound according to embodiment 2 or 3 wherein X1 and X8 are joined by a disulphide bond or a methylene bridge, and X1 and X14 are joined by an amide bond between the alpha amine of X1 and the alpha carboxylic group of X14.
6. The bicyclic compound according to any one of embodiments 1-5 wherein said compound comprises 9-14 amino acid residues.
7. The bicyclic compound according to any one of the preceding embodiments wherein X1 is selected from the group consisting of Cys, HCys and Pen.
8. The bicyclic compound according to any one of the preceding embodiments wherein X2 is selected from the group consisting of THAZ and Pro.
9. The bicyclic compound according to any one of the preceding embodiments wherein X3 is His.
10. The bicyclic compound according to any one of the preceding embodiments wherein X4 is D-Phe.
11. The bicyclic compound according to any one of the preceding embodiments wherein X5 is selected from the group consisting of Arg, His, Dab, and Dap.
12. The bicyclic compound according to any one of the preceding embodiments wherein X6 is Trp.
13. The bicyclic compound according to any one of the preceding embodiments wherein X7 is absent.
14. The bicyclic compound according to any one of the preceding embodiments wherein X8 is selected from the group consisting of Cys, HCys, and Pen.
15. The bicyclic compound according to any one of the preceding embodiments wherein X9 is selected from the group consisting of D-Lys, Lys, Arg, and D-Arg.
16. The bicyclic compound according to any one of the preceding embodiments wherein X10 is selected from the group consisting of Ala, Glu, and D-Ala.
17. The bicyclic compound according to any one of the preceding embodiments wherein X11 is selected from the group consisting of Lys, D-Lys, Arg, and D-Arg.
18. The bicyclic compound according to any one of the preceding embodiments wherein X12 is selected from the group consisting of Ala, D-Ala, and Glu.
19. The bicyclic compound according to any one of the preceding embodiments wherein X13 is selected from the group consisting of Arg and Trp.
20. The bicyclic compound according to any one of the preceding embodiments wherein X14 is absent.
21. The bicyclic compound according to any one of embodiments 1-6 selected from the group consisting of:

| | Chem. (SEQ ID NO) and |
|---|---|
| Structure | Amino acid sequence |
| 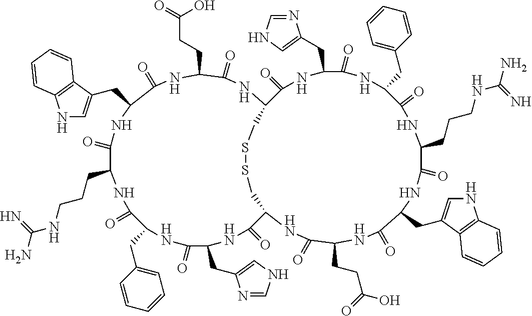 [SMILES: N=C(N)NCCC[C@@H]1NC(=O)[C@@H](Cc2ccccc2)NC(=O)[C@H](Cc2c[nH]cn2)NC(=O)[C@@H]2CSSC[C@H](NC(=O)[C@H](CCC(=O)O)NC(=O)[C@H](Cc3c[nH]c4ccccc34)NC1=O)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N[C@@H](CCC(=O)O)C(=O)N2] | Chem. 1 (SEQ ID NO: 1) c[c[Cys-His-D-Phe-Arg-Trp-Glu-Cys]- His-D-Phe-Arg-Trp-Glu] |
| 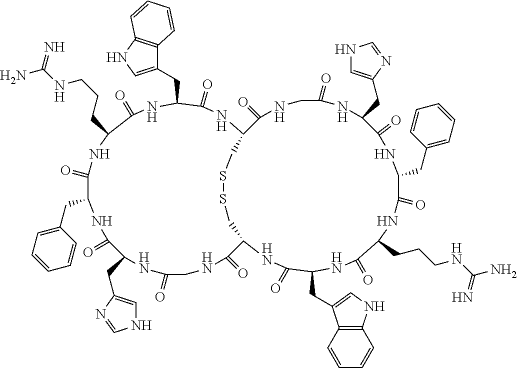 [SMILES: N=C(N)NCCC[C@@H]1NC(=O)[C@@H](Cc2ccccc2)NC(=O)[C@H](Cc2c[nH]cn2)NC(=O)CNC(=O)[C@@H]2CSSC[C@H](NC(=O)[C@H](Cc3c[nH]c4ccccc34)NC1=O)C(=O)NCC(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 2 (SEQ ID NO: 2) c[c[Cys-Gly-His-D-Phe-Arg-Trp-Cys]- Gly-His-D-Phe-Arg-Trp] |
| 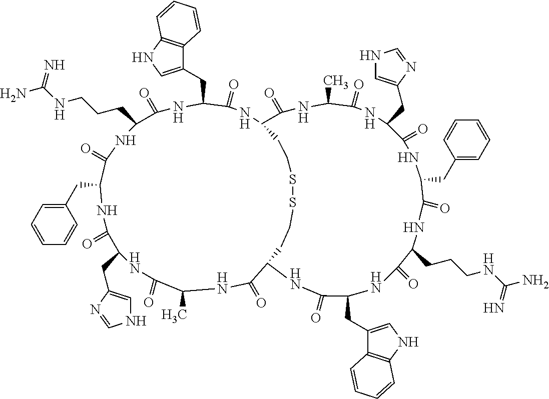 | Chem. 3 (SEQ ID NO: 3) c[c[HCys-Ala-His-D-Phe-Arg-Trp- HCys]-Ala-His-D-Phe-Arg-Trp] |
| 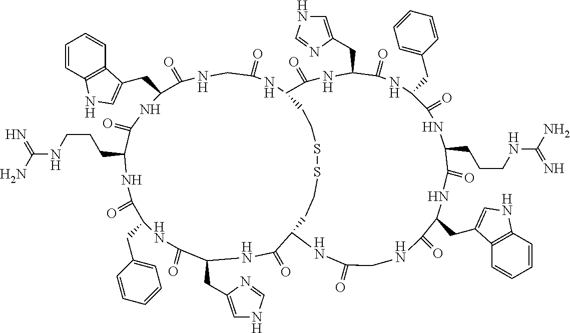 | Chem. 4 (SEQ ID NO: 4) c[c[Cys-His-D-Phe-Arg-Trp-Gly-Cys]- His-D-Phe-Arg-Trp-Gly] |
| 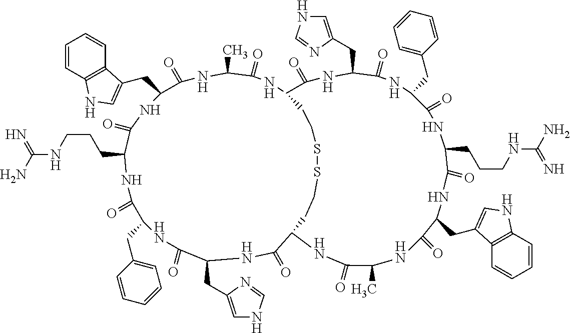 | Chem. 5 (SEQ ID NO: 5) c[c[Cys-His-D-Phe-Arg-Trp-Ala-Cys]- His-D-Phe-Arg-Trp-Ala] |
| 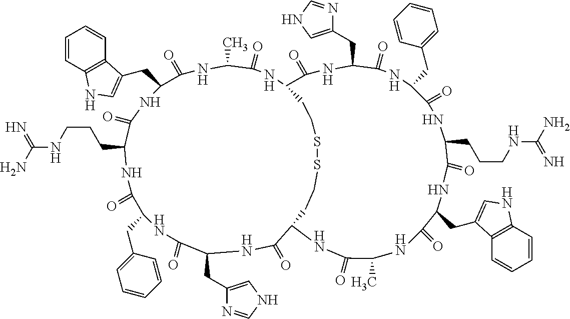 | Chem. 6 (SEQ ID NO: 6) c[c[Cys-His-D-Phe-Arg-Trp-D-Ala- Cys]-His-D-Phe-Arg-Trp-D-Ala] |
| 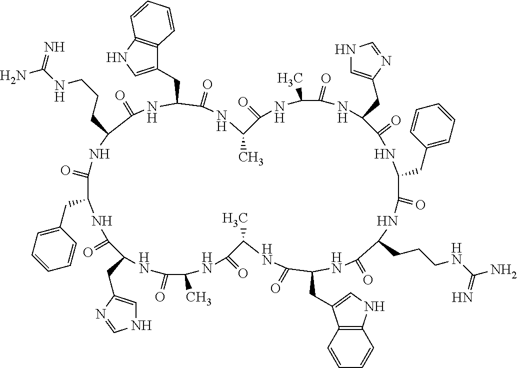 [SMILES: C[C@@H]1NC(=O)[C@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC(=O)[C@H](C)NC(=O)[C@H](C)NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC1=O] | Chem. 7 (SEQ ID NO: 7) c[Ala-Ala-His-D-Phe-Arg-Trp-Ala-Ala- His-D-Phe-Arg-Trp] |
| 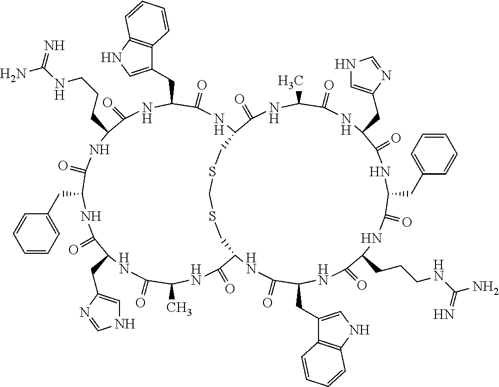 [SMILES: C[C@@H]1NC(=O)[C@@H]2CSCSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 8 (SEQ ID NO: 8) c[c[Cys-Ala-His-D-Phe-Arg-Trp-Cys]- Ala-His-D-Phe-Arg-Trp] Note: methylene bridge |
| 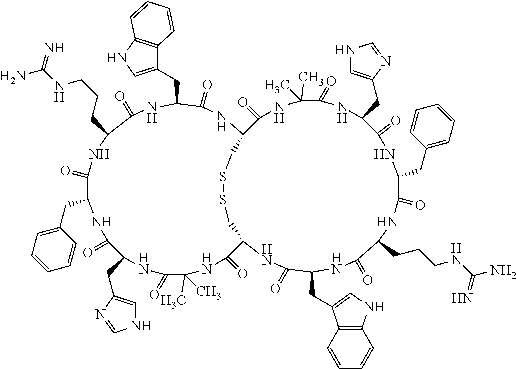 [SMILES: CC1(C)NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)NC(C)(C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 9 (SEQ ID NO: 9) c[c[Cys-Aib-His-D-Phe-Arg-Trp-Cys]- Aib-His-D-Phe-Arg-Trp] |
| 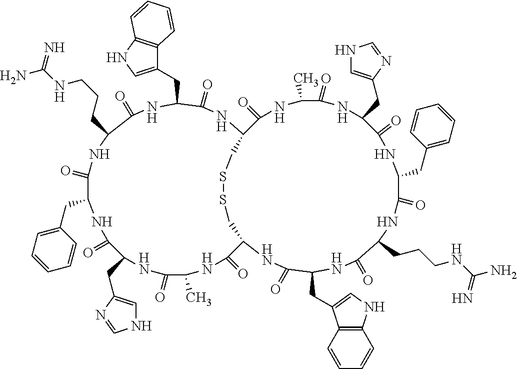 [SMILES: C[C@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 10 (SEQ ID NO: 10) c[c[Cys-D-Ala-His-D-Phe-Arg-Trp- Cys]-D-Ala-His-D-Phe-Arg-Trp] |
| 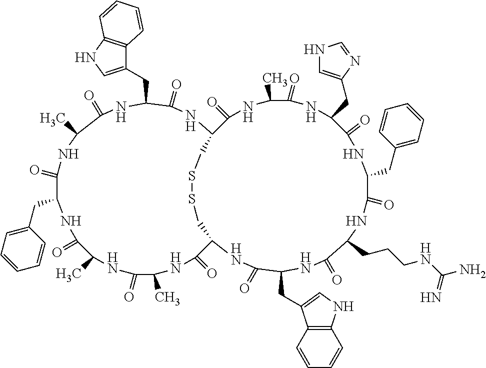 [SMILES: C[C@@H]1NC(=O)[C@H](C)NC(=O)[C@@H]2CSSC[C@H](NC(=O)[C@H](Cc3c[nH]c4ccccc34)NC(=O)[C@H](C)NC(=O)[C@@H](Cc3ccccc3)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 11 (SEQ ID NO: 11) c[[Cys-Ala-His-D-Phe-Arg-Trp-Cys]- Ala-Ala-D-Phe-Ala-Trp] |
| 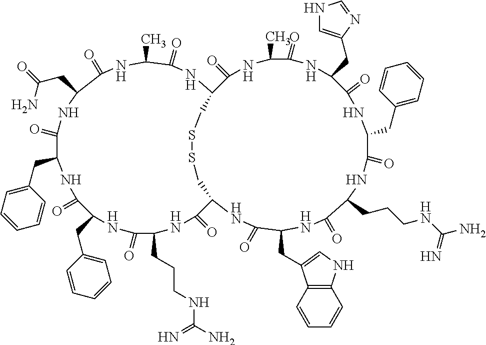 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](CCCNC(=N)N)C(=O)N[C@@H](Cc1ccccc1)C(=O)N[C@@H](Cc1ccccc1)C(=O)N[C@@H](CC(N)=O)C(=O)N[C@@H](C)C(=O)N2] | Chem. 12 (SEQ ID NO: 12) c[c[Cys-Ala-His-D-Phe-Arg-Trp-Cys]- Arg-Phe-Phe-Asn-Ala] |
| 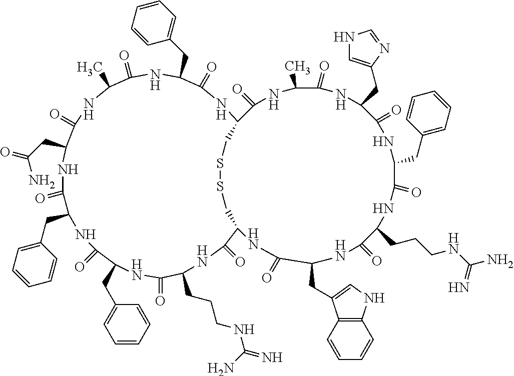 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](CCCNC(=N)N)C(=O)N[C@@H](Cc1ccccc1)C(=O)N[C@@H](Cc1ccccc1)C(=O)N[C@@H](CC(N)=O)C(=O)N[C@@H](C)C(=O)N[C@@H](Cc1ccccc1)C(=O)N2] | Chem. 13 (SEQ ID NO: 13) c[c[Cys-Ala-His-D-Phe-Arg-Trp-Cys]- Arg-Phe-Phe-Asn-Ala-Phe] |
| 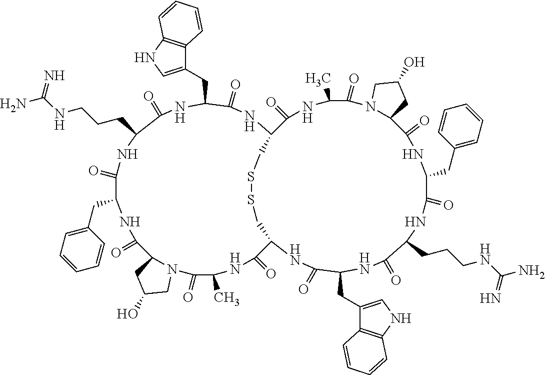 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@@H]3C[C@@H](O)CN3C1=O)C(=O)N[C@@H](C)C(=O)N1C[C@H](O)C[C@H]1C(=O)N[C@H](Cc1ccccc1)C(=O)N[C@@H](CCCNC(=N)N)C(=O)N[C@@H](Cc1c[nH]c3ccccc13)C(=O)N2] | Chem. 14 (SEQ ID NO: 14) c[c[Cys-Ala-Hyp-D-Phe-Arg-Trp- Cys]-Ala-Hyp-D-Phe-Arg-Trp] |
| 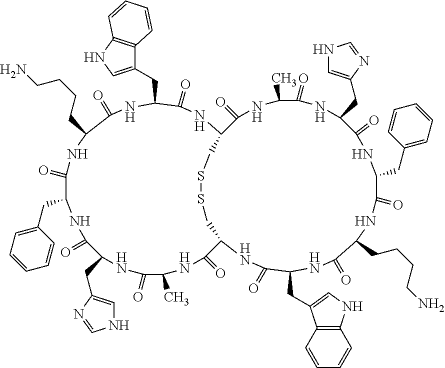 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CCCCN)C(=O)N[C@@H](Cc1c[nH]c3ccccc13)C(=O)N2] | Chem. 15 (SEQ ID NO: 15) c[c[Cys-Ala-His-D-Phe-Lys-Trp-Cys]- Ala-His-D-Phe-Lys-Trp] |
| 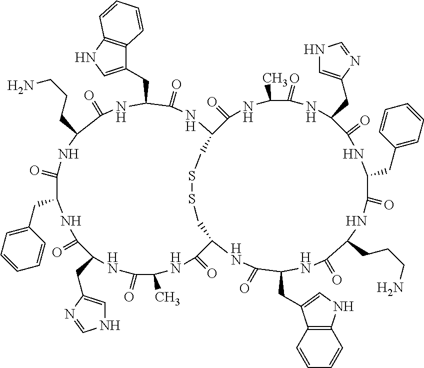 [SMILES: C[C@@H]1NC(=O)[C@@H]2CSSC[C@H](NC(=O)[C@H](Cc3c[nH]c4ccccc34)NC(=O)[C@H](CCCN)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CCCN)C(=O)N[C@@H](Cc1c[nH]c3ccccc13)C(=O)N2] | Chem. 16 (SEQ ID NO: 16) c[c[Cys-Ala-His-D-Phe-Orn-Trp-Cys]- Ala-His-D-Phe-Orn-Trp] |
| 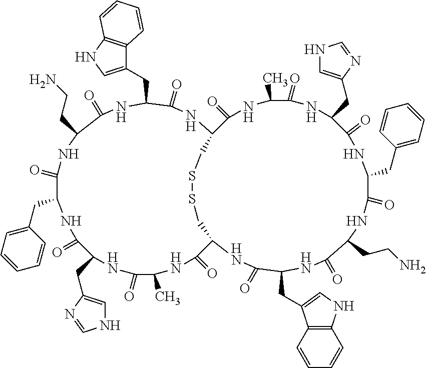 [SMILES: C[C@@H]1NC(=O)[C@@H]2CSSC[C@H](NC(=O)[C@H](Cc3c[nH]c4ccccc34)NC(=O)[C@H](CCN)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CCN)C(=O)N[C@@H](Cc1c[nH]c3ccccc13)C(=O)N2] | Chem. 17 (SEQ ID NO: 17) c[c[Cys-Ala-His-D-Phe-Dab-Trp-Cys]- Ala-His-D-Phe-Dab-Trp] |
| 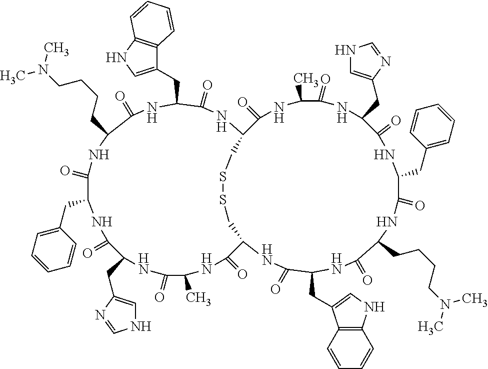 | Chem. 18 (SEQ ID NO: 18) c[c[Cys-Ala-His-D-Phe-Lys(Me)₂-Trp- Cys]-Ala-His-D-Phe-Lys(Me)₂-Trp] |
| 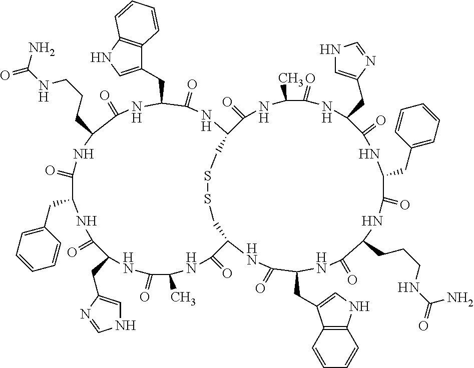 [SMILES: C[C@@H]1NC(=O)[C@@H]2CSSC[C@H](NC(=O)[C@H](Cc3c[nH]c4ccccc34)NC(=O)[C@H](CCCNC(N)=O)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CCCNC(N)=O)C(=O)N[C@@H](Cc1c[nH]c3ccccc13)C(=O)N2] | Chem. 19 (SEQ ID NO: 19) c[c[Cys-Ala-His-D-Phe-Cit-Trp-Cys]- Ala-His-D-Phe-Cit-Trp] |
| 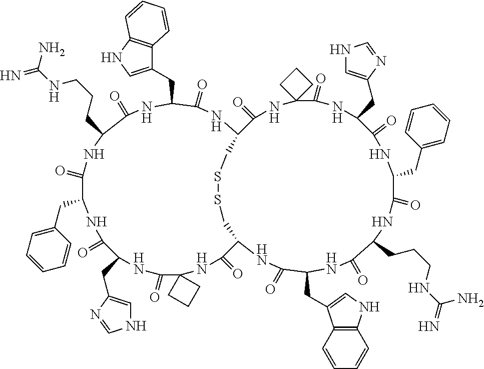 [SMILES: N=C(N)NCCC[C@@H]1NC(=O)[C@@H](Cc2ccccc2)NC(=O)[C@H](Cc2c[nH]cn2)NC(=O)C2(CCC2)NC(=O)[C@@H]2CSSC[C@H](NC(=O)[C@H](Cc3c[nH]c4ccccc34)NC1=O)C(=O)NC1(CCC1)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 20 (SEQ ID NO: 20) c[c[Cys-ACBC-His-D-Phe-Arg-Trp- Cys]-ACBC-His-D-Phe-Arg-Trp] |
| 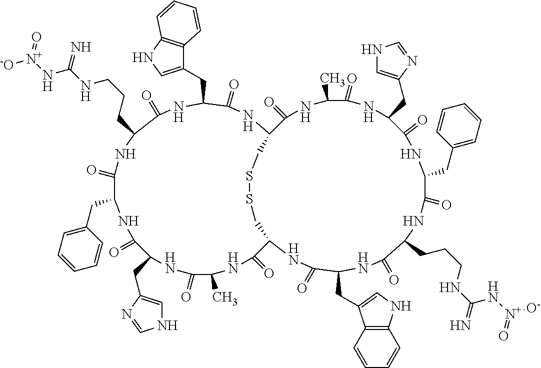 | Chem. 21 (SEQ ID NO: 21) c[c[Cys-Ala-His-D-Phe-Arg(NO₂)-Trp- Cys]-Ala-His-D-Phe-Arg(NO₂)-Trp] |
| 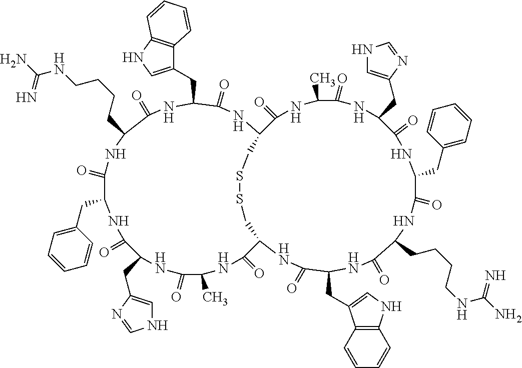 [SMILES: C[C@@H]1NC(=O)[C@@H]2CSSC[C@H](NC(=O)[C@H](Cc3c[nH]c4ccccc34)NC(=O)[C@H](CCCCNC(=N)N)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CCCCNC(=N)N)C(=O)N[C@@H](Cc1c[nH]c3ccccc13)C(=O)N2] | Chem. 22 (SEQ ID NO: 22) c[c[Cys-Ala-His-D-Phe-HArg-Trp- Cys]-Ala-His-D-Phe-HArg-Trp] |
| 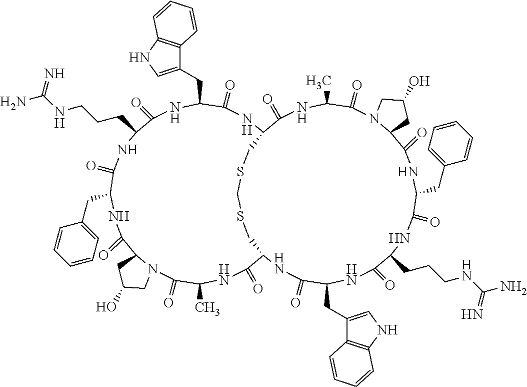 [SMILES: C[C@@H]1NC(=O)[C@@H]2CSCSC[C@H](NC(=O)[C@H](Cc3c[nH]c4ccccc34)NC(=O)[C@H](CCCNC(=N)N)NC(=O)[C@@H](Cc3ccccc3)NC(=O)[C@@H]3C[C@@H](O)CN3C1=O)C(=O)N[C@@H](C)C(=O)N1C[C@H](O)C[C@H]1C(=O)N[C@H](Cc1ccccc1)C(=O)N[C@@H](CCCNC(=N)N)C(=O)N[C@@H](Cc1c[nH]c3ccccc13)C(=O)N2] | Chem. 23 (SEQ ID NO: 23) c[c[Cys-Ala-Hyp-D-Phe-Arg-Trp- Cys]-Ala-Hyp-D-Phe-Arg-Trp] Note: methylene bridge |
| 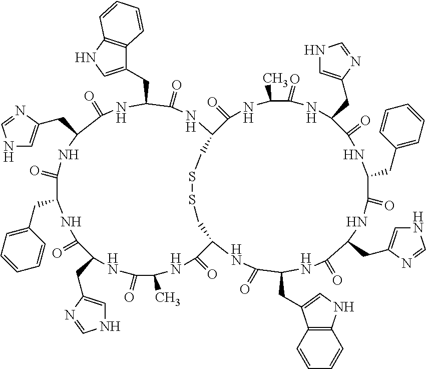 [SMILES: C[C@@H]1NC(=O)[C@@H]2CSSC[C@H](NC(=O)[C@H](Cc3c[nH]c4ccccc34)NC(=O)[C@H](Cc3c[nH]cn3)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 24 (SEQ ID NO: 24) c[c[Cys-Ala-His-D-Phe-His-Trp-Cys]- Ala-His-D-Phe-His-Trp] |
| 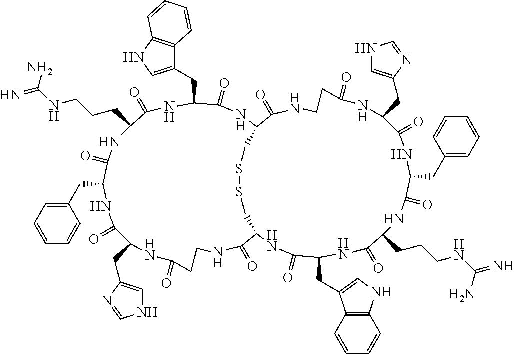 [SMILES: N=C(N)NCCC[C@@H]1NC(=O)[C@@H](Cc2ccccc2)NC(=O)[C@H](Cc2c[nH]cn2)NC(=O)CCNC(=O)[C@@H]2CSSC[C@H](NC(=O)[C@H](Cc3c[nH]c4ccccc34)NC1=O)C(=O)NCCC(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 25 (SEQ ID NO: 25) c[c[Cys-βAla-His-D-Phe-Arg-Trp- Cys]-βAla-His-D-Phe-Arg-Trp] |
| 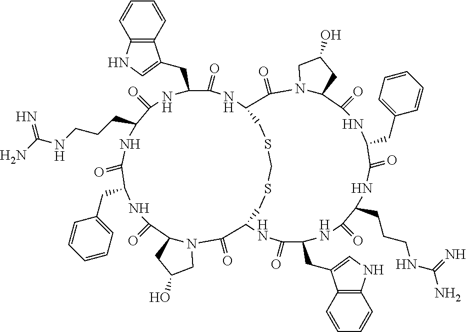 [SMILES: N=C(N)NCCC[C@@H]1NC(=O)[C@@H](Cc2ccccc2)NC(=O)[C@@H]2C[C@@H](O)CN2C(=O)[C@@H]2CSCSC[C@H](NC(=O)[C@H](Cc3c[nH]c4ccccc34)NC1=O)C(=O)N1C[C@H](O)C[C@H]1C(=O)N[C@H](Cc1ccccc1)C(=O)N[C@@H](CCCNC(=N)N)C(=O)N[C@@H](Cc1c[nH]c3ccccc13)C(=O)N2] | Chem. 26 (SEQ ID NO: 26) c[c[Cys-Hyp-D-Phe-Arg-Trp-Cys]- Hyp-D-Phe-Arg-Trp] Note: methylene bridge |
| 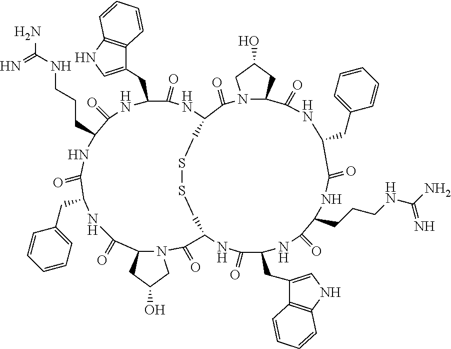 [SMILES: N=C(N)NCCC[C@@H]1NC(=O)[C@@H](Cc2ccccc2)NC(=O)[C@@H]2C[C@@H](O)CN2C(=O)[C@@H]2CSSC[C@H](NC(=O)[C@H](Cc3c[nH]c4ccccc34)NC1=O)C(=O)N1C[C@H](O)C[C@H]1C(=O)N[C@H](Cc1ccccc1)C(=O)N[C@@H](CCCNC(=N)N)C(=O)N[C@@H](Cc1c[nH]c3ccccc13)C(=O)N2] | Chem. 27 (SEQ ID NO: 27) c[c[Cys-Hyp-D-Phe-Arg-Trp-Cys]- Hyp-D-Phe-Arg-Trp] |
| 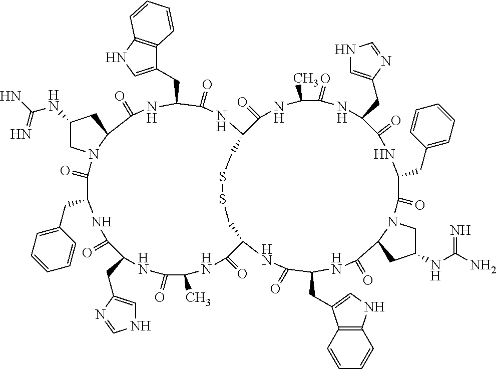 [SMILES: C[C@@H]1NC(=O)[C@@H]2CSSC[C@H](NC(=O)[C@H](Cc3c[nH]c4ccccc34)NC(=O)[C@@H]3C[C@@H](NC(=N)N)CN3C(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N1C[C@H](NC(=N)N)C[C@H]1C(=O)N[C@@H](Cc1c[nH]c3ccccc13)C(=O)N2] | Chem. 28 (SEQ ID NO: 28) c[c[Cys-Ala-His-D-Phe-4trans- GuaPro-Trp-Cys]-Ala-His-D-Phe- 4trans-GuaPro-Trp] |
| 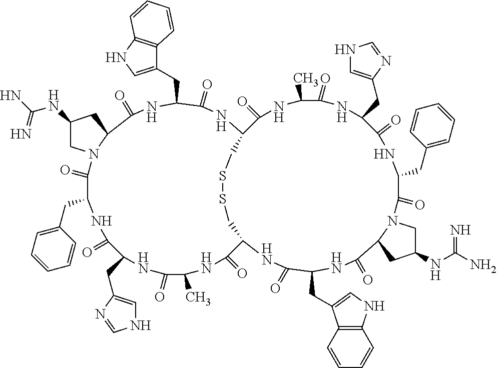 [SMILES: C[C@@H]1NC(=O)[C@@H]2CSSC[C@H](NC(=O)[C@H](Cc3c[nH]c4ccccc34)NC(=O)[C@@H]3C[C@H](NC(=N)N)CN3C(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N1C[C@@H](NC(=N)N)C[C@H]1C(=O)N[C@@H](Cc1c[nH]c3ccccc13)C(=O)N2] | Chem. 29 (SEQ ID NO: 29) c[c[Cys-Ala-His-D-Phe-4cis-GuaPro- Trp-Cys]-Ala-His-D-Phe-4cis-GuaPro- Trp] |
| 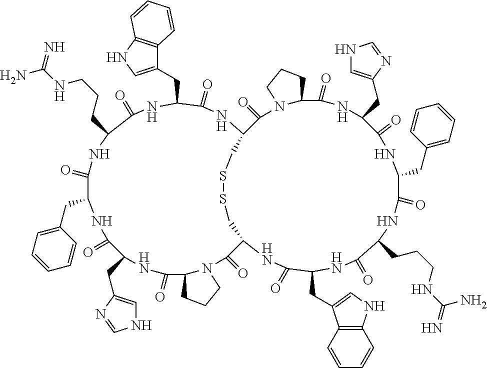 [SMILES: N=C(N)NCCC[C@@H]1NC(=O)[C@@H](Cc2ccccc2)NC(=O)[C@H](Cc2c[nH]cn2)NC(=O)[C@@H]2CCCN2C(=O)[C@@H]2CSSC[C@H](NC(=O)[C@H](Cc3c[nH]c4ccccc34)NC1=O)C(=O)N1CCC[C@H]1C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 30 (SEQ ID NO: 30) c[c[Cys-Pro-His-D-Phe-Arg-Trp-Cys]- Pro-His-D-Phe-Arg-Trp] |
| 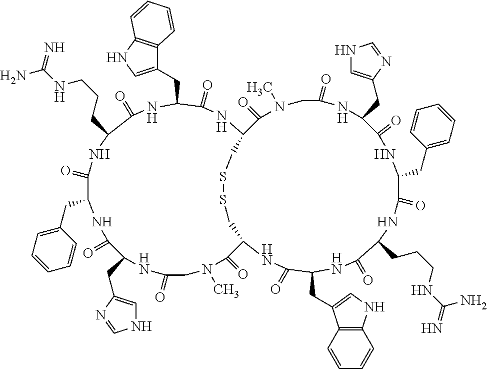 [SMILES: CN1CC(=O)N[C@@H](Cc2c[nH]cn2)C(=O)N[C@H](Cc2ccccc2)C(=O)N[C@@H](CCCNC(=N)N)C(=O)N[C@@H](Cc2c[nH]c3ccccc23)C(=O)N[C@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC(=O)CN(C)C2=O)C1=O] | Chem. 31 (SEQ ID NO: 31) c[c[Cys-Sar-His-D-Phe-Arg-Trp-Cys]- Sar-His-D-Phe-Arg-Trp] |
| 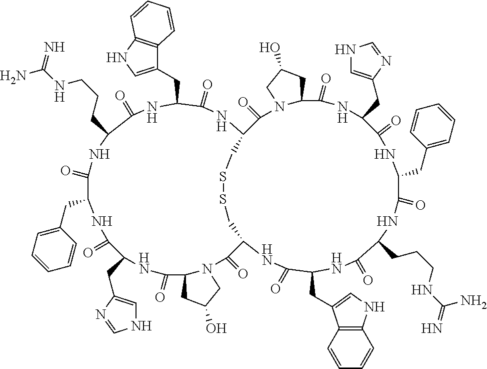 [SMILES: N=C(N)NCCC[C@@H]1NC(=O)[C@@H](Cc2ccccc2)NC(=O)[C@H](Cc2c[nH]cn2)NC(=O)[C@@H]2C[C@@H](O)CN2C(=O)[C@@H]2CSSC[C@H](NC(=O)[C@H](Cc3c[nH]c4ccccc34)NC1=O)C(=O)N1C[C@H](O)C[C@H]1C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 32 (SEQ ID NO: 32) c[c[Cys-Hyp-His-D-Phe-Arg-Trp- Cys]-Hyp-His-D-Phe-Arg-Trp] |
| 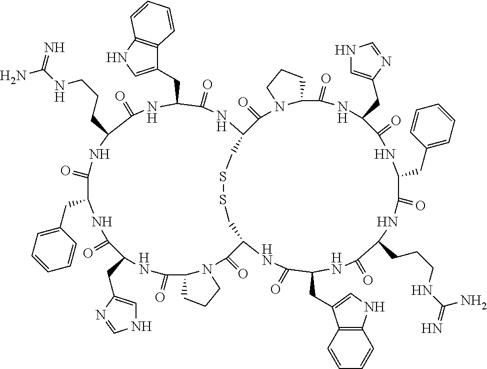 [SMILES: N=C(N)NCCC[C@@H]1NC(=O)[C@@H](Cc2ccccc2)NC(=O)[C@H](Cc2c[nH]cn2)NC(=O)[C@H]2CCCN2C(=O)[C@@H]2CSSC[C@H](NC(=O)[C@H](Cc3c[nH]c4ccccc34)NC1=O)C(=O)N1CCC[C@@H]1C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 33 (SEQ ID NO: 33) c[c[Cys-D-Pro-His-D-Phe-Arg-Trp- Cys]-D-Pro-His-D-Phe-Arg-Trp] |
| 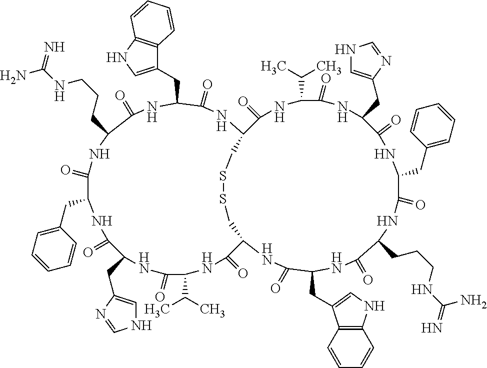 [SMILES: CC(C)[C@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@H](C(C)C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 34 (SEQ ID NO: 34) c[c[Cys-D-Val-His-D-Phe-Arg-Trp- Cys]-D-Val-His-D-Phe-Arg-Trp] |
| 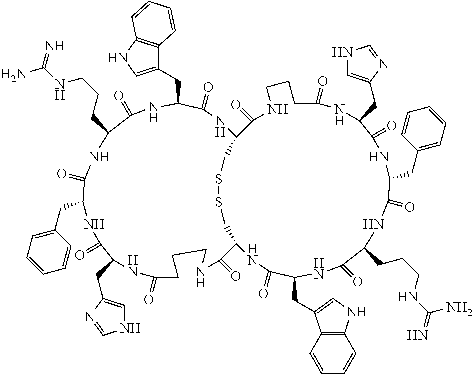 [SMILES: N=C(N)NCCC[C@@H]1NC(=O)[C@@H](Cc2ccccc2)NC(=O)[C@H](Cc2c[nH]cn2)NC(=O)CCCNC(=O)[C@@H]2CSSC[C@H](NC(=O)[C@H](Cc3c[nH]c4ccccc34)NC1=O)C(=O)NCCCC(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 35 (SEQ ID NO: 35) c[c[Cys-GABA-His-D-Phe-Arg-Trp- Cys]-GABA-His-D-Phe-Arg-Trp] |
| 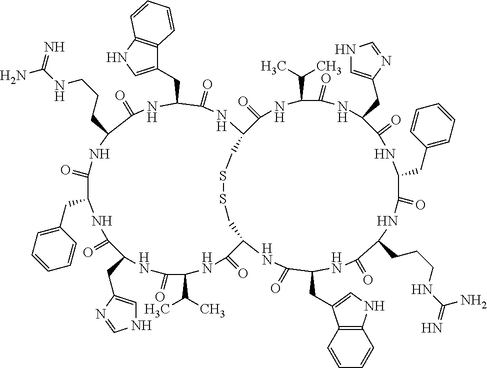 [SMILES: CC(C)[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](C(C)C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 36 (SEQ ID NO: 36) c[c[Cys-Val-His-D-Phe-Arg-Trp-Cys]- Val-His-D-Phe-Arg-Trp] |
| 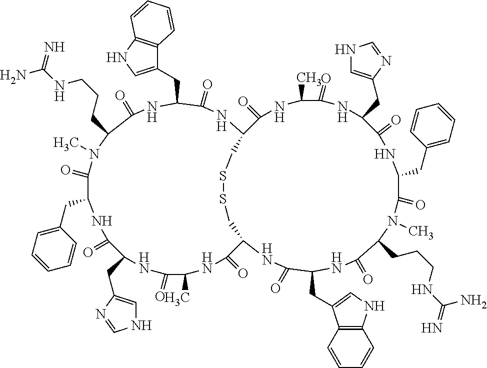 [SMILES: C[C@@H]1NC(=O)[C@@H]2CSSC[C@H](NC(=O)[C@H](Cc3c[nH]c4ccccc34)NC(=O)[C@H](CCCNC(=N)N)N(C)C(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N(C)[C@@H](CCCNC(=N)N)C(=O)N[C@@H](Cc1c[nH]c3ccccc13)C(=O)N2] | Chem. 37 (SEQ ID NO: 37) c[c[Cys-Ala-His-D-Phe-N-MeArg-Trp- Cys]-Ala-His-D-Phe-N-MeArg-Trp] |
| 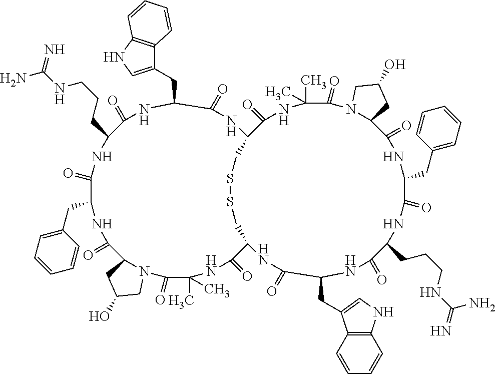 [SMILES: CC1(C)NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@@H]3C[C@@H](O)CN3C1=O)C(=O)NC(C)(C)C(=O)N1C[C@H](O)C[C@H]1C(=O)N[C@H](Cc1ccccc1)C(=O)N[C@@H](CCCNC(=N)N)C(=O)N[C@@H](Cc1c[nH]c3ccccc13)C(=O)N2] | Chem. 38 (SEQ ID NO: 38) c[c[Cys-Aib-Hyp-D-Phe-Arg-Trp- Cys]-Aib-Hyp-D-Phe-Arg-Trp] |
| 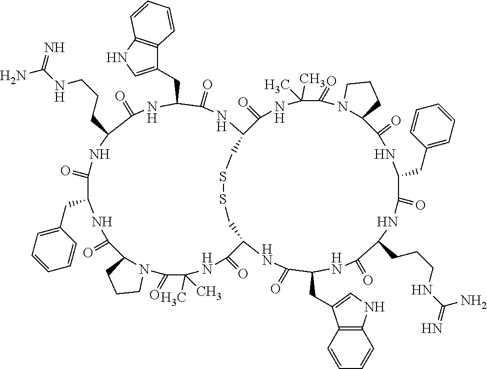 [SMILES: CC1(C)NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@@H]3CCCN3C1=O)C(=O)NC(C)(C)C(=O)N1CCC[C@H]1C(=O)N[C@H](Cc1ccccc1)C(=O)N[C@@H](CCCNC(=N)N)C(=O)N[C@@H](Cc1c[nH]c3ccccc13)C(=O)N2] | Chem. 39 (SEQ ID NO: 39) c[c[Cys-Aib-Pro-D-Phe-Arg-Trp-Cys]- Aib-Pro-D-Phe-Arg-Trp] |
| 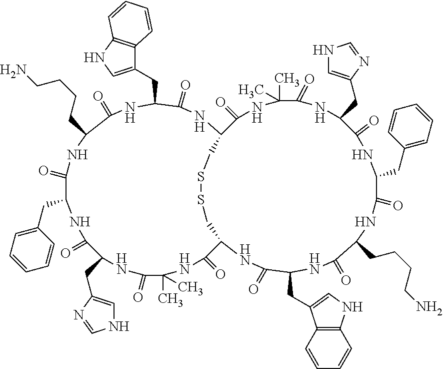 [SMILES: CC1(C)NC(=O)[C@@H]2CSSC[C@H](NC(=O)[C@H](Cc3c[nH]c4ccccc34)NC(=O)[C@H](CCCCN)NC(=O)[C@@H](Cc3ccccc3)NC(=O)[C@H](Cc3c[nH]cn3)NC1=O)C(=O)NC(C)(C)C(=O)N[C@@H](Cc1c[nH]cn1)C(=O)N[C@H](Cc1ccccc1)C(=O)N[C@@H](CCCCN)C(=O)N[C@@H](Cc1c[nH]c3ccccc13)C(=O)N2] | Chem. 40 (SEQ ID NO: 40) c[c[Cys-Aib-His-D-Phe-Lys-Trp-Cys]- Aib-His-D-Phe-Lys-Trp] |
| 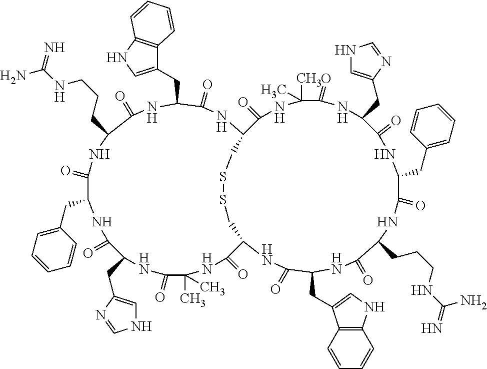 [SMILES: CC1(C)NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)NC(C)(C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 41 (SEQ ID NO: 41) c[c[Cys-Aib-His-D-Phe-Cit-Trp-Cys]- Aib-His-D-Phe-Cit-Trp] |
| 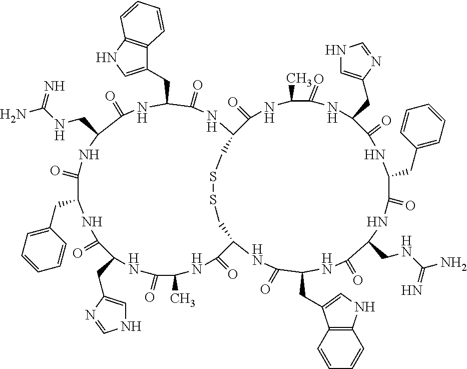 [SMILES: C[C@@H]1NC(=O)[C@@H]2CSSC[C@H](NC(=O)[C@H](Cc3c[nH]c4ccccc34)NC(=O)[C@H](CNC(=N)N)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CNC(=N)N)C(=O)N[C@@H](Cc1c[nH]c3ccccc13)C(=O)N2] | Chem. 42 (SEQ ID NO: 42) c[c[Cys-Ala-His-D-Phe-AGP-Trp- Cys]-Ala-His-D-Phe-AGP-Trp] |
| 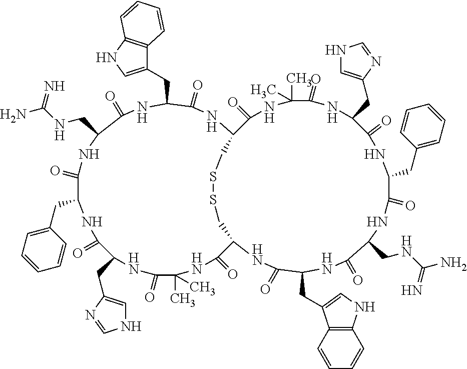 [SMILES: CC1(C)NC(=O)[C@@H]2CSSC[C@H](NC(=O)[C@H](Cc3c[nH]c4ccccc34)NC(=O)[C@H](CNC(=N)N)NC(=O)[C@@H](Cc3ccccc3)NC(=O)[C@H](Cc3c[nH]cn3)NC1=O)C(=O)NC(C)(C)C(=O)N[C@@H](Cc1c[nH]cn1)C(=O)N[C@H](Cc1ccccc1)C(=O)N[C@@H](CNC(=N)N)C(=O)N[C@@H](Cc1c[nH]c3ccccc13)C(=O)N2] | Chem. 43 (SEQ ID NO: 43) c[c[Cys-Aib-His-D-Phe-AGP-Trp- Cys]-Aib-His-D-Phe-AGP-Trp] |
| 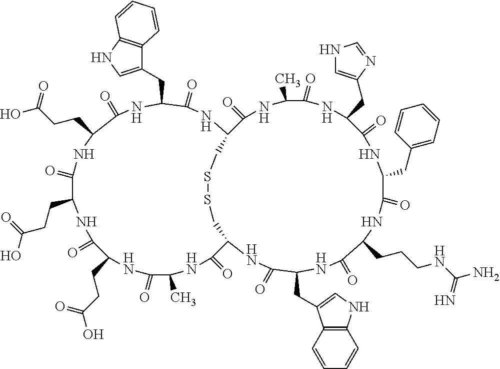 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](CCC(=O)O)C(=O)N[C@@H](CCC(=O)O)C(=O)N[C@@H](CCC(=O)O)C(=O)N[C@@H](Cc1c[nH]c3ccccc13)C(=O)N2] | Chem. 44 (SEQ ID NO: 44) c[c[Cys-Ala-His-D-Phe-Arg-Trp-Cys]- Ala-Glu-Glu-Glu-Trp] |
| 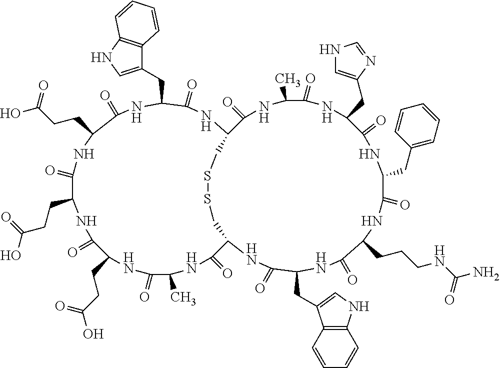 [SMILES: C[C@@H]1NC(=O)[C@@H]2CSSC[C@H](NC(=O)[C@H](Cc3c[nH]c4ccccc34)NC(=O)[C@H](CCCNC(N)=O)NC(=O)[C@@H](Cc3ccccc3)NC(=O)[C@H](Cc3c[nH]cn3)NC1=O)C(=O)N[C@@H](C)C(=O)N[C@@H](CCC(=O)O)C(=O)N[C@@H](CCC(=O)O)C(=O)N[C@@H](CCC(=O)O)C(=O)N[C@@H](Cc1c[nH]c3ccccc13)C(=O)N2] | Chem. 45 (SEQ ID NO: 45) c[c[Cys-Ala-His-D-Phe-Cit-Trp-Cys]- Ala-Glu-Glu-Glu-Trp] |
| 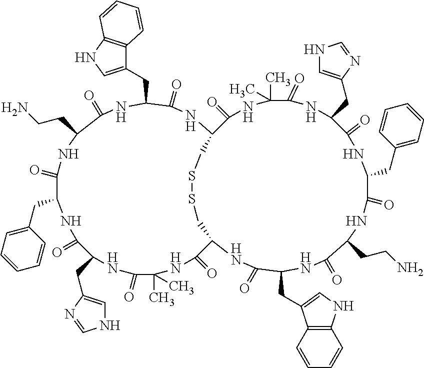 [SMILES: CC1(C)NC(=O)[C@@H]2CSSC[C@H](NC(=O)[C@H](Cc3c[nH]c4ccccc34)NC(=O)[C@H](CCN)NC(=O)[C@@H](Cc3ccccc3)NC(=O)[C@H](Cc3c[nH]cn3)NC1=O)C(=O)NC(C)(C)C(=O)N[C@@H](Cc1c[nH]cn1)C(=O)N[C@H](Cc1ccccc1)C(=O)N[C@@H](CCN)C(=O)N[C@@H](Cc1c[nH]c3ccccc13)C(=O)N2] | Chem. 46 (SEQ ID NO: 46) c[c[Cys-Aib-His-D-Phe-Dab-Trp- Cys]-Aib-His-D-Phe-Dab-Trp] |
| 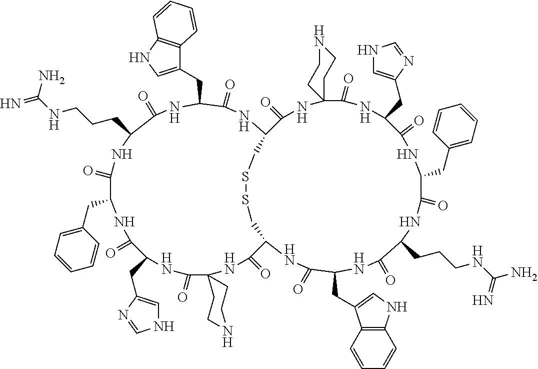 [SMILES: N=C(N)NCCC[C@@H]1NC(=O)[C@@H](Cc2ccccc2)NC(=O)[C@H](Cc2c[nH]cn2)NC(=O)C2(CCNCC2)NC(=O)[C@@H]2CSSC[C@H](NC(=O)[C@H](Cc3c[nH]c4ccccc34)NC1=O)C(=O)NC1(CCNCC1)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 47 (SEQ ID NO: 47) c[c[Cys-ACP-His-D-Phe-Arg-Trp- Cys]-ACP-His-D-Phe-Arg-Trp] |
| 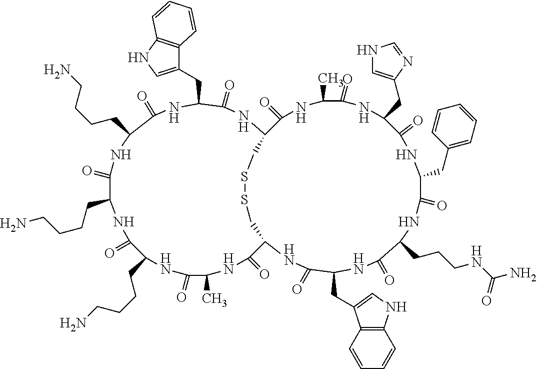 [SMILES: C[C@@H]1NC(=O)[C@@H]2CSSC[C@H](NC(=O)[C@H](Cc3c[nH]c4ccccc34)NC(=O)[C@H](CCCNC(N)=O)NC(=O)[C@@H](Cc3ccccc3)NC(=O)[C@H](Cc3c[nH]cn3)NC1=O)C(=O)N[C@@H](C)C(=O)N[C@@H](CCCCN)C(=O)N[C@@H](CCCCN)C(=O)N[C@@H](CCCCN)C(=O)N[C@@H](Cc1c[nH]c3ccccc13)C(=O)N2] | Chem. 48 (SEQ ID NO: 48) c[c[Cys-Ala-His-D-Phe-Cit-Trp-Cys]- Ala-Lys-Lys-Lys-Trp] |
| 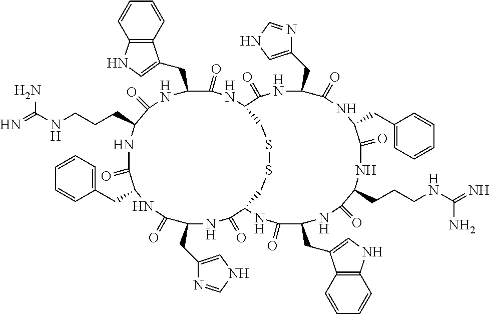 [SMILES: N=C(N)NCCC[C@@H]1NC(=O)[C@@H](Cc2ccccc2)NC(=O)[C@H](Cc2c[nH]cn2)NC(=O)[C@@H]2CSSC[C@H](NC(=O)[C@H](Cc3c[nH]c4ccccc34)NC1=O)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 49 (SEQ ID NO: 49) c[c[Cys-His-D-Phe-Arg-Trp-Cys]-His- D-Phe-Arg-Trp] |
| 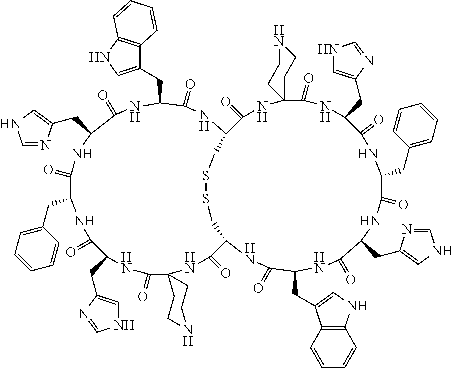 [SMILES: O=C1N[C@H]2CSSC[C@H](NC(=O)[C@H](Cc3c[nH]c4ccccc34)NC(=O)[C@H](Cc3c[nH]cn3)NC(=O)[C@@H](Cc3ccccc3)NC(=O)[C@H](Cc3c[nH]cn3)NC(=O)C3(CCNCC3)NC2=O)C(=O)NC2(CCNCC2)C(=O)N[C@@H](Cc2c[nH]cn2)C(=O)N[C@H](Cc2ccccc2)C(=O)N[C@@H](Cc2c[nH]cn2)C(=O)N[C@H]1Cc1c[nH]c2ccccc12] | Chem. 50 (SEQ ID NO: 50) c[c[Cys-ACP-His-D-Phe-His-Trp-Cys]- ACP-His-D-Phe-His-Trp] |
| 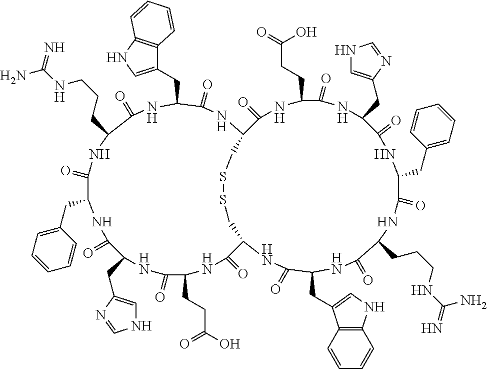 [SMILES: N=C(N)NCCC[C@@H]1NC(=O)[C@@H](Cc2ccccc2)NC(=O)[C@H](Cc2c[nH]cn2)NC(=O)[C@H](CCC(=O)O)NC(=O)[C@@H]2CSSC[C@H](NC(=O)[C@H](Cc3c[nH]c4ccccc34)NC1=O)C(=O)N[C@@H](CCC(=O)O)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 51 (SEQ ID NO: 51) c[c[Cys-Glu-His-D-Phe-Arg-Trp-Cys]- Glu-His-D-Phe-Arg-Trp] |
| 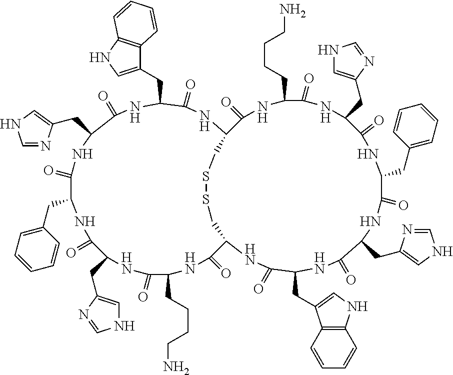 [SMILES: NCCCC[C@@H]1NC(=O)[C@@H]2CSSC[C@H](NC(=O)[C@H](Cc3c[nH]c4ccccc34)NC(=O)[C@H](Cc3c[nH]cn3)NC(=O)[C@@H](Cc3ccccc3)NC(=O)[C@H](Cc3c[nH]cn3)NC1=O)C(=O)N[C@@H](CCCCN)C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 52 (SEQ ID NO: 52) c[c[Cys-Lys-His-D-Phe-His-Trp-Cys]- Lys-His-D-Phe-His-Trp] |
| 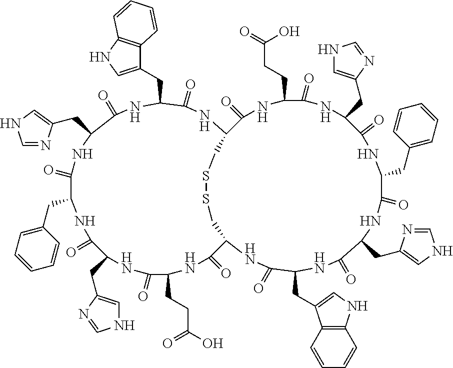 [SMILES: O=C(O)CC[C@@H]1NC(=O)[C@@H]2CSSC[C@H](NC(=O)[C@H](Cc3c[nH]c4ccccc34)NC(=O)[C@H](Cc3c[nH]cn3)NC(=O)[C@@H](Cc3ccccc3)NC(=O)[C@H](Cc3c[nH]cn3)NC1=O)C(=O)N[C@@H](CCC(=O)O)C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 53 (SEQ ID NO: 53) c[c[Cys-Glu-His-D-Phe-His-Trp-Cys]- Glu-His-D-Phe-His-Trp] |
| 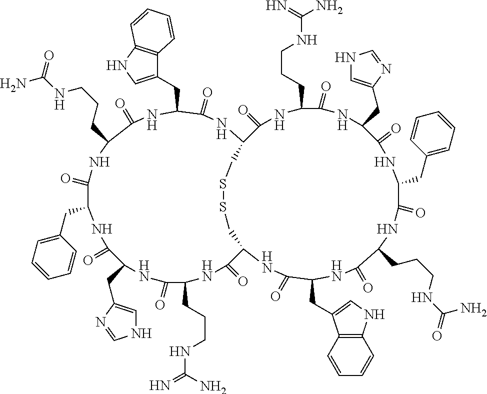 [SMILES: N=C(N)NCCC[C@@H]1NC(=O)[C@@H]2CSSC[C@H](NC(=O)[C@H](Cc3c[nH]c4ccccc34)NC(=O)[C@H](CCCNC(N)=O)NC(=O)[C@@H](Cc3ccccc3)NC(=O)[C@H](Cc3c[nH]cn3)NC1=O)C(=O)N[C@@H](CCCNC(=N)N)C(=O)N[C@@H](Cc1c[nH]cn1)C(=O)N[C@H](Cc1ccccc1)C(=O)N[C@@H](CCCNC(N)=O)C(=O)N[C@@H](Cc1c[nH]c3ccccc13)C(=O)N2] | Chem. 54 (SEQ ID NO: 54) c[c[Cys-Arg-His-D-Phe-Cit-Trp-Cys]- Arg-His-D-Phe-Cit-Trp] |
| 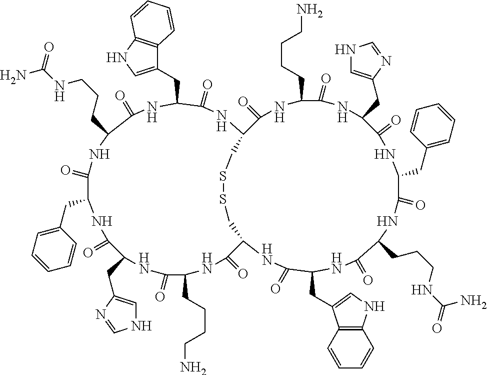 [SMILES: NCCCC[C@@H]1NC(=O)[C@@H]2CSSC[C@H](NC(=O)[C@H](Cc3c[nH]c4ccccc34)NC(=O)[C@H](CCCNC(N)=O)NC(=O)[C@@H](Cc3ccccc3)NC(=O)[C@H](Cc3c[nH]cn3)NC1=O)C(=O)N[C@@H](CCCCN)C(=O)N[C@@H](Cc1c[nH]cn1)C(=O)N[C@H](Cc1ccccc1)C(=O)N[C@@H](CCCNC(N)=O)C(=O)N[C@@H](Cc1c[nH]c3ccccc13)C(=O)N2] | Chem. 55 (SEQ ID NO: 55) c[c[Cys-Lys-His-D-Phe-Cit-Trp-Cys]- Lys-His-D-Phe-Cit-Trp] |
| 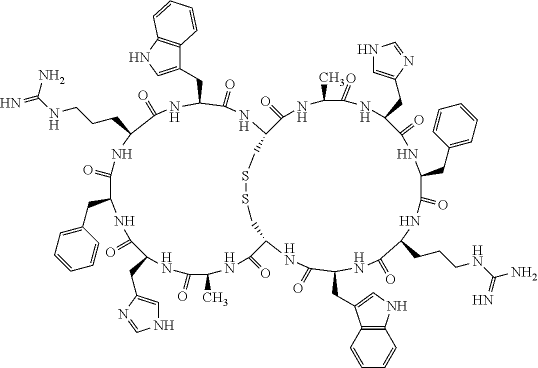 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@@H](Cc1ccccc1)C(=O)N[C@@H](CCCNC(=N)N)C(=O)N[C@@H](Cc1c[nH]c3ccccc13)C(=O)N2] | Chem. 56 (SEQ ID NO: 56) c[c[Cys-Ala-His-Phe-Arg-Trp-Cys]- Ala-His-Phe-Arg-Trp] |
| 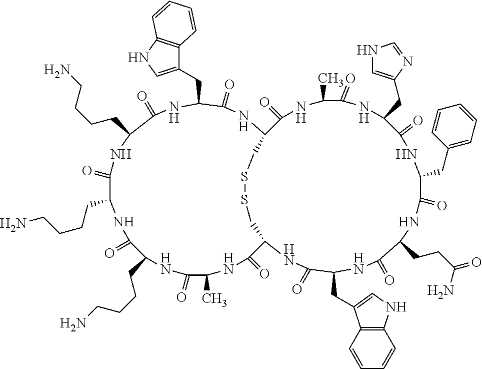 [SMILES: C[C@@H]1NC(=O)[C@@H]2CSSC[C@H](NC(=O)[C@H](Cc3c[nH]c4ccccc34)NC(=O)[C@H](CCC(N)=O)NC(=O)[C@@H](Cc3ccccc3)NC(=O)[C@H](Cc3c[nH]cn3)NC1=O)C(=O)N[C@@H](C)C(=O)N[C@@H](CCCCN)C(=O)N[C@H](CCCCN)C(=O)N[C@@H](CCCCN)C(=O)N[C@@H](Cc1c[nH]c3ccccc13)C(=O)N2] | Chem. 57 (SEQ ID NO: 57) c[c[Cys-Ala-His-D-Phe-Gln-Trp-Cys]- Ala-Lys-D-Lys-Lys-Trp] |
| 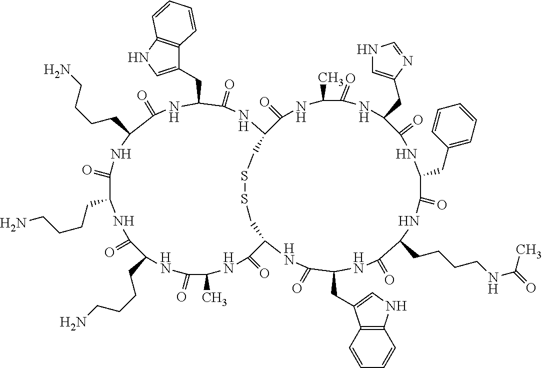 [SMILES: CC(=O)NCCCC[C@@H]1NC(=O)[C@@H](Cc2ccccc2)NC(=O)[C@H](Cc2c[nH]cn2)NC(=O)[C@H](C)NC(=O)[C@@H]2CSSC[C@H](NC(=O)[C@H](Cc3c[nH]c4ccccc34)NC1=O)C(=O)N[C@@H](C)C(=O)N[C@@H](CCCCN)C(=O)N[C@H](CCCCN)C(=O)N[C@@H](CCCCN)C(=O)N[C@@H](Cc1c[nH]c3ccccc13)C(=O)N2] | Chem. 58 (SEQ ID NO: 58) c[c[Cys-Ala-His-D-Phe-AcLys-Trp- Cys]-Ala-Lys-D-Lys-Lys-Trp] |
| 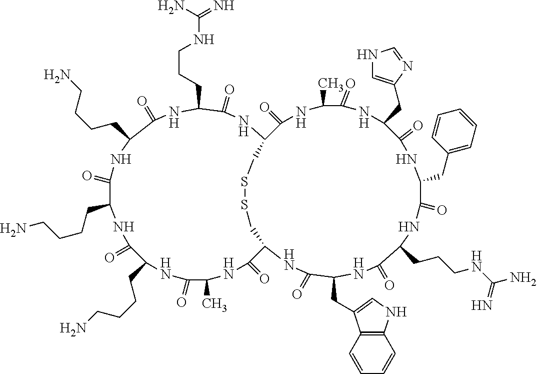 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](CCCCN)C(=O)N[C@@H](CCCCN)C(=O)N[C@@H](CCCCN)C(=O)N[C@@H](CCCNC(=N)N)C(=O)N2] | Chem. 59 (SEQ ID NO: 59) c[c[Cys-Ala-His-D-Phe-Arg-Trp-Cys]- Ala-Lys-Lys-Lys-Arg] |
| 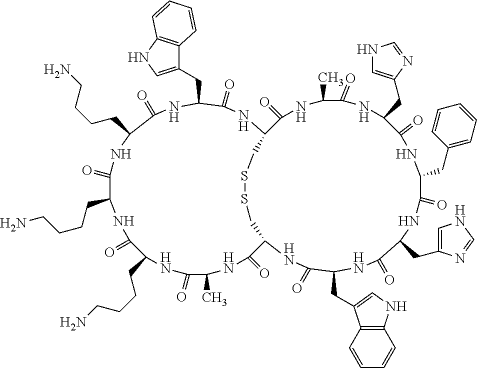 [SMILES: C[C@@H]1NC(=O)[C@@H]2CSSC[C@H](NC(=O)[C@H](Cc3c[nH]c4ccccc34)NC(=O)[C@H](Cc3c[nH]cn3)NC(=O)[C@@H](Cc3ccccc3)NC(=O)[C@H](Cc3c[nH]cn3)NC1=O)C(=O)N[C@@H](C)C(=O)N[C@@H](CCCCN)C(=O)N[C@@H](CCCCN)C(=O)N[C@@H](CCCCN)C(=O)N[C@@H](Cc1c[nH]c3ccccc13)C(=O)N2] | Chem. 60 (SEQ ID NO: 60) c[c[Cys-Ala-His-D-Phe-His-Trp-Cys]- Ala-Lys-Lys-Lys-Trp] |
| 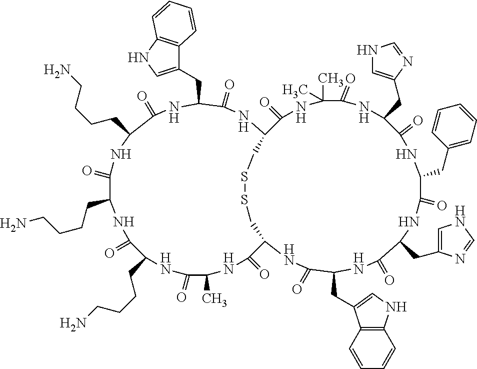 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)NC(C)(C)C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 61 (SEQ ID NO: 61) c[c[Cys-Aib-His-D-Phe-His-Trp-Cys]- Ala-Lys-Lys-Lys-Trp] |
| 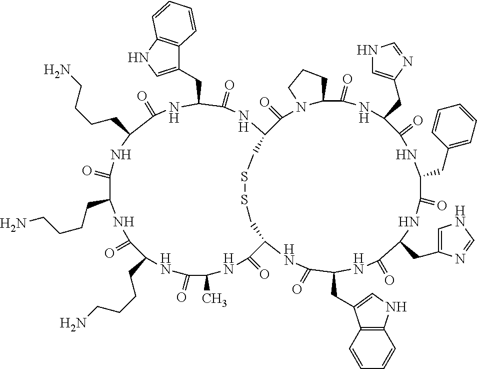 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N1CCC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 62 (SEQ ID NO: 62) c[c[Cys-Pro-His-D-Phe-His-Trp-Cys]- Ala-Lys-Lys-Lys-Trp] |
| 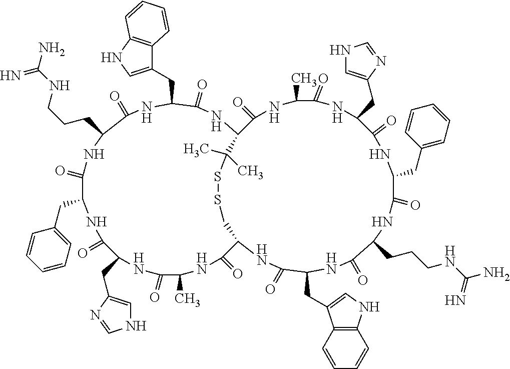 [SMILES: C[C@@H]1NC(=O)[C@@H]2CSSC(C)(C)[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 63 (SEQ ID NO: 63) c[c[Pen-Ala-His-D-Phe-Arg-Trp-Cys]- Ala-His-D-Phe-Arg-Trp] |
| 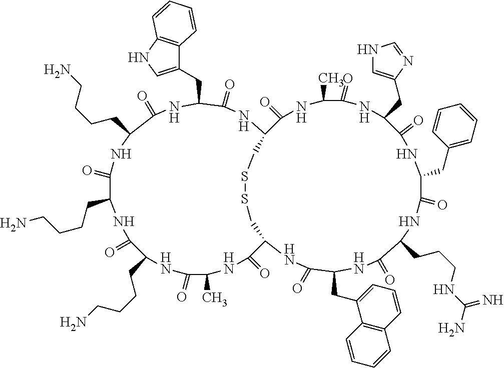 [SMILES: C[C@@H]1NC(=O)[C@@H]2CSSC[C@H](NC(=O)[C@H](Cc3cccc4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](CCCCN)C(=O)N[C@@H](CCCCN)C(=O)N[C@@H](CCCCN)C(=O)N[C@@H](Cc1c[nH]c3ccccc13)C(=O)N2] | Chem. 64 (SEQ ID NO: 64) c[c[Cys-Ala-His-D-Phe-Arg-1-Nal- Cys]-Ala-Lys-Lys-Lys-Trp] |
| 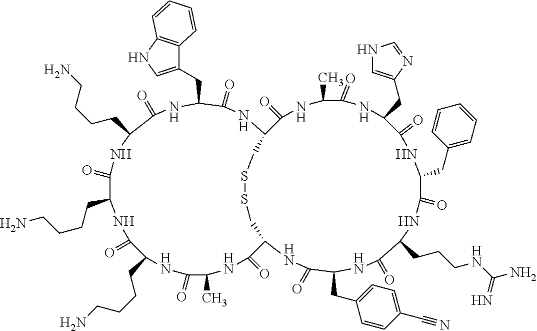 [SMILES: C[C@@H]1NC(=O)[C@@H]2CSSC[C@H](NC(=O)[C@H](Cc3ccc(C#N)cc3)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](CCCCN)C(=O)N[C@@H](CCCCN)C(=O)N[C@@H](CCCCN)C(=O)N[C@@H](Cc1c[nH]c3ccccc13)C(=O)N2] | Chem. 65 (SEQ ID NO: 65) c[c[Cys-Ala-His-D-Phe-Arg-4-CN- Phe-Cys]-Ala-Lys-Lys-Lys-Trp] |
| 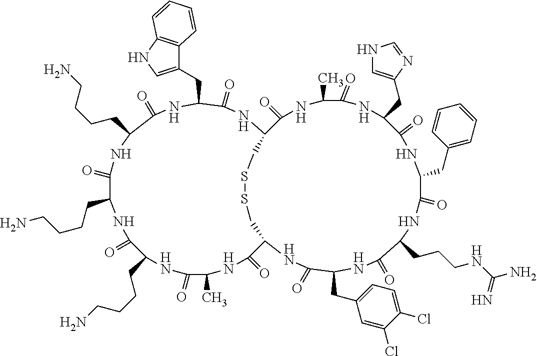 [SMILES: C[C@@H]1NC(=O)[C@@H]2CSSC[C@H](NC(=O)[C@H](Cc3ccc(Cl)c(Cl)c3)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](CCCCN)C(=O)N[C@@H](CCCCN)C(=O)N[C@@H](CCCCN)C(=O)N[C@@H](Cc1c[nH]c3ccccc13)C(=O)N2] | Chem. 66 (SEQ ID NO: 66) c[c[Cys-Ala-His-D-Phe-Arg-3,4-DiCl- Phe-Cys]-Ala-Lys-Lys-Lys-Trp] |
| 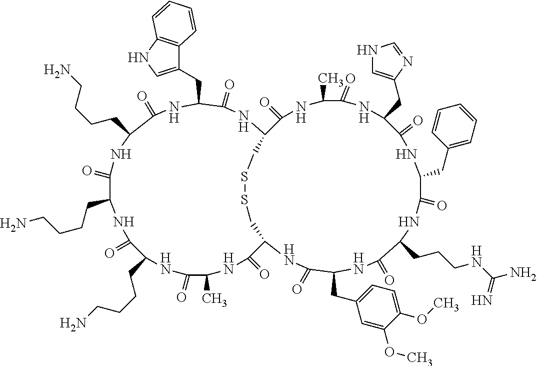 [SMILES: COc1ccc(C[C@@H]2NC(=O)[C@H](CCCNC(=N)N)NC(=O)[C@@H](Cc3ccccc3)NC(=O)[C@H](Cc3c[nH]cn3)NC(=O)[C@H](C)NC(=O)[C@@H]3CSSC[C@H](NC2=O)C(=O)N[C@@H](C)C(=O)N[C@@H](CCCCN)C(=O)N[C@@H](CCCCN)C(=O)N[C@@H](CCCCN)C(=O)N[C@@H](Cc2c[nH]c4ccccc24)C(=O)N3)cc1OC] | Chem. 67 (SEQ ID NO: 67) c[c[Cys-Ala-His-D-Phe-Arg-3,4- DiMeo-Phe-Cys]-Ala-Lys-Lys-Lys- Trp] |
| 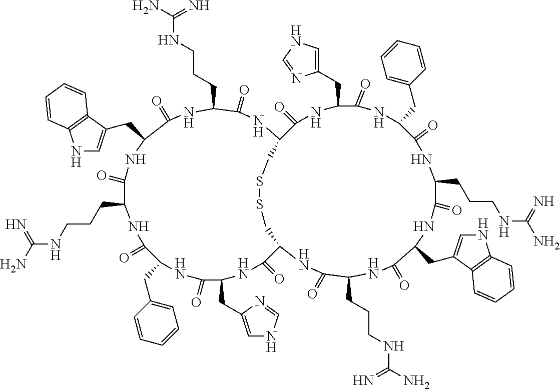 [SMILES: N=C(N)NCCC[C@@H]1NC(=O)[C@H](Cc2c[nH]c3ccccc23)NC(=O)[C@H](CCCNC(=N)N)NC(=O)[C@@H](Cc2ccccc2)NC(=O)[C@H](Cc2c[nH]cn2)NC(=O)[C@@H]2CSSC[C@H](NC1=O)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N[C@@H](CCCNC(=N)N)C(=O)N2] | Chem. 68 (SEQ ID NO: 68) c[c[Cys-His-D-Phe-Arg-Trp-Arg-Cys]- His-D-Phe-Arg-Trp-Arg] |
| 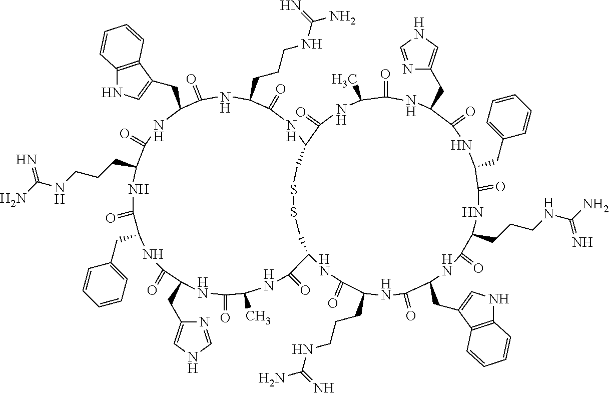 [SMILES: C[C@@H]1NC(=O)[C@@H]2CSSC[C@H](NC(=O)[C@H](CCCNC(=N)N)NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N[C@@H](CCCNC(=N)N)C(=O)N2] | Chem. 69 (SEQ ID NO: 69) c[c[Cys-Ala-His-D-Phe-Arg-Trp-Arg- Cys]-Ala-His-D-Phe-Arg-Trp-Arg] |
| 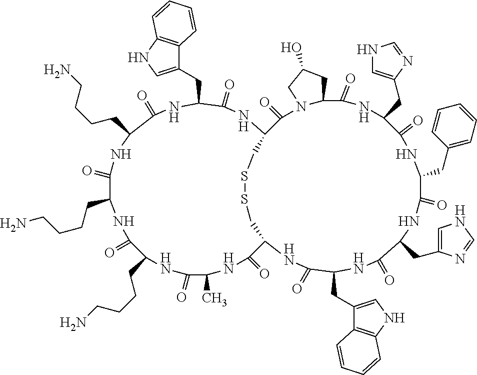 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N1C[C@H](O)C[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 70 (SEQ ID NO: 70) c[c[Cys-Hyp-His-D-Phe-His-Trp-Cys]- Ala-Lys-Lys-Lys-Trp] |
| 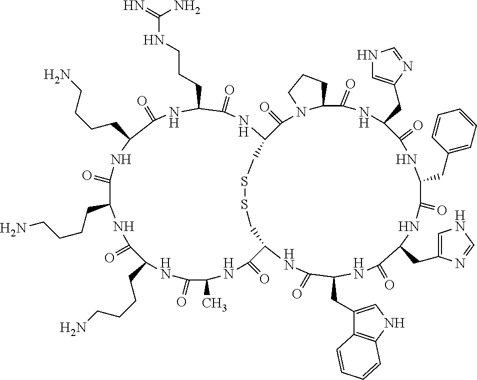 [SMILES: C[C@@H]1NC(=O)[C@@H]2CSSC[C@H](NC(=O)[C@H](CCCNC(=N)N)NC(=O)[C@H](CCCCN)NC(=O)[C@H](CCCCN)NC(=O)[C@H](CCCCN)NC1=O)C(=O)N1CCC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 71 (SEQ ID NO: 71) c[c[Cys-Pro-His-D-Phe-His-Trp-Cys]- Ala-Lys-Lys-Lys-Arg] |
| 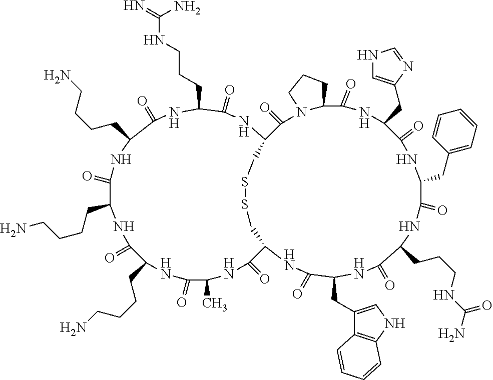 [SMILES: C[C@@H]1NC(=O)[C@@H]2CSSC[C@H](NC(=O)[C@H](CCCNC(=N)N)NC(=O)[C@H](CCCCN)NC(=O)[C@H](CCCCN)NC(=O)[C@H](CCCCN)NC1=O)C(=O)N1CCC[C@H]1C(=O)N[C@@H](Cc1c[nH]cn1)C(=O)N[C@H](Cc1ccccc1)C(=O)N[C@@H](CCCNC(N)=O)C(=O)N[C@@H](Cc1c[nH]c3ccccc13)C(=O)N2] | Chem. 72 (SEQ ID NO: 72) c[c[Cys-Pro-His-D-Phe-Cit-Trp-Cys]- Ala-Lys-Lys-Lys-Arg] |
| 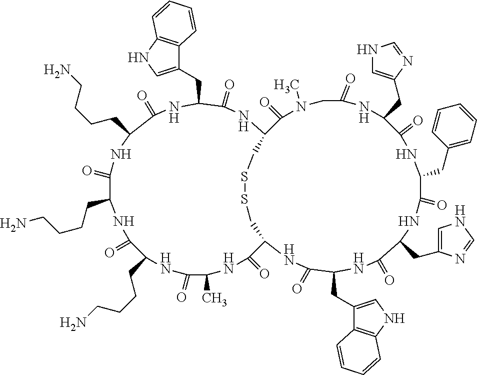 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N(C)CC(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 73 (SEQ ID NO: 73) c[c[Cys-Sar-His-D-Phe-His-Trp-Cys]- Ala-Lys-Lys-Lys-Trp] |
| 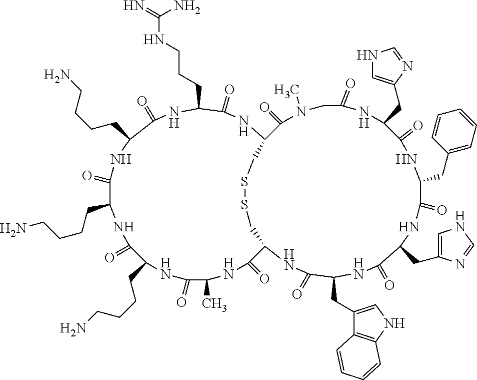 [SMILES: C[C@@H]1NC(=O)[C@@H]2CSSC[C@H](NC(=O)[C@H](CCCNC(=N)N)NC(=O)[C@H](CCCCN)NC(=O)[C@H](CCCCN)NC(=O)[C@H](CCCCN)NC1=O)C(=O)N(C)CC(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 74 (SEQ ID NO: 74) c[c[Cys-Sar-His-D-Phe-His-Trp-Cys]- Ala-Lys-Lys-Lys-Arg] |
| 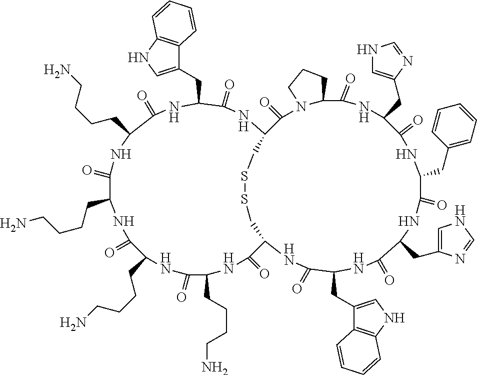 [SMILES: NCCCC[C@@H]1NC(=O)[C@H](CCCCN)NC(=O)[C@@H]2CSSC[C@H](NC(=O)[C@H](Cc3c[nH]c4ccccc34)NC(=O)[C@H](CCCCN)NC(=O)[C@H](CCCCN)NC1=O)C(=O)N1CCC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 75 (SEQ ID NO: 75) c[c[Cys-Pro-His-D-Phe-His-Trp-Cys]- Lys-Lys-Lys-Lys-Trp] |
| 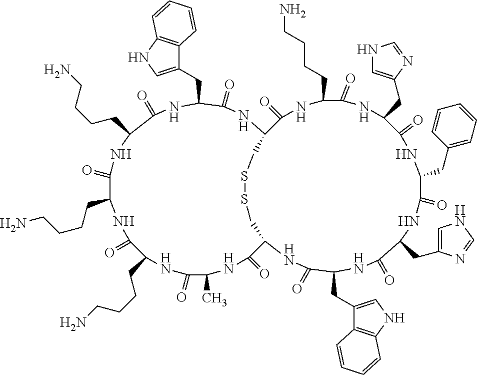 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N[C@@H](CCCCN)C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 76 (SEQ ID NO: 76) c[c[Cys-Lys-His-D-Phe-His-Trp-Cys]- Ala-Lys-Lys-Lys-Trp] |
| 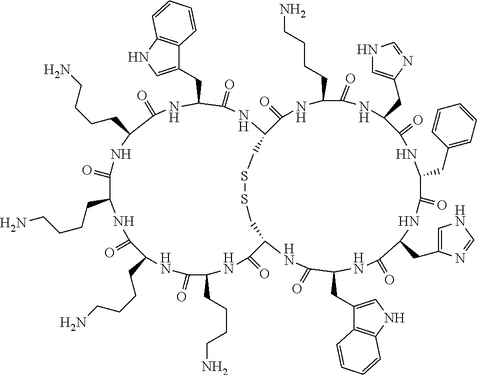 [SMILES: NCCCC[C@@H]1NC(=O)[C@H](CCCCN)NC(=O)[C@H](CCCCN)NC(=O)[C@@H]2CSSC[C@H](NC(=O)[C@H](Cc3c[nH]c4ccccc34)NC(=O)[C@H](CCCCN)NC1=O)C(=O)N[C@@H](CCCCN)C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 77 (SEQ ID NO: 77) c[c[Cys-Lys-His-D-Phe-His-Trp-Cys]- Lys-Lys-Lys-Lys-Trp] |
| 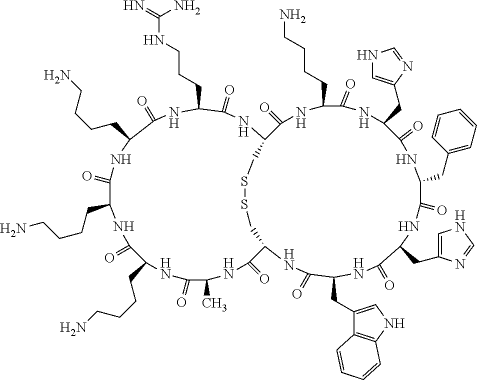 [SMILES: C[C@@H]1NC(=O)[C@@H]2CSSC[C@H](NC(=O)[C@H](CCCNC(=N)N)NC(=O)[C@H](CCCCN)NC(=O)[C@H](CCCCN)NC(=O)[C@H](CCCCN)NC1=O)C(=O)N[C@@H](CCCCN)C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 78 (SEQ ID NO: 78) c[c[Cys-Lys-His-D-Phe-His-Trp-Cys]- Ala-Lys-Lys-Lys-Arg] |
| 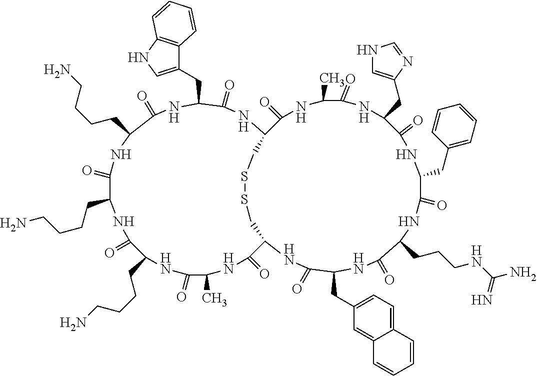 [SMILES: C[C@@H]1NC(=O)[C@@H]2CSSC[C@H](NC(=O)[C@H](Cc3ccc4ccccc4c3)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](CCCCN)C(=O)N[C@@H](CCCCN)C(=O)N[C@@H](CCCCN)C(=O)N[C@@H](Cc1c[nH]c3ccccc13)C(=O)N2] | Chem. 79 (SEQ ID NO: 79) c[c[Cys-Ala-His-D-Phe-Arg-2-Nal- Cys]-Ala-Lys-Lys-Lys-Trp] |
| 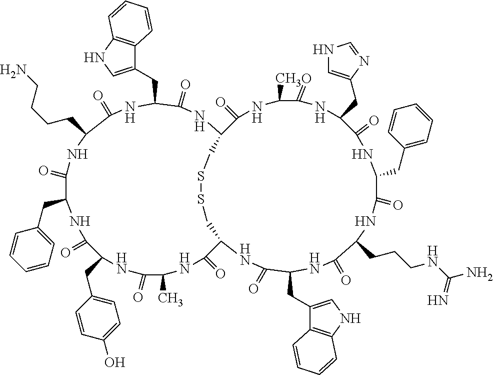 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](Cc1ccc(O)cc1)C(=O)N[C@@H](Cc1ccccc1)C(=O)N[C@@H](CCCCN)C(=O)N[C@@H](Cc1c[nH]c3ccccc13)C(=O)N2] | Chem. 80 (SEQ ID NO: 80) c[c[Cys-Ala-His-D-Phe-Arg-Trp-Cys]- Ala-Tyr-Phe-Lys-Trp] |
| 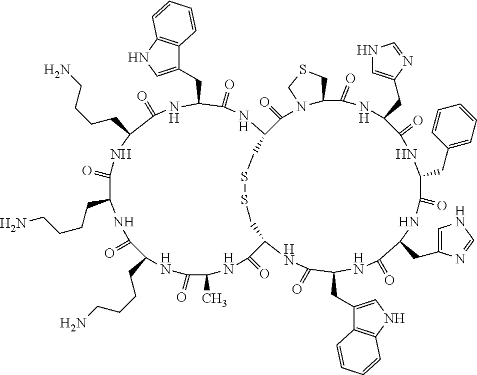 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 81 (SEQ ID NO: 81) c[c[Cys-THAZ-His-D-Phe-His-Trp- Cys]-Ala-Lys-Lys-Lys-Trp] |
| 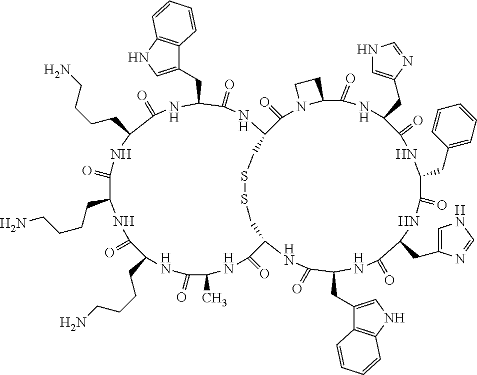 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N1CC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 82 (SEQ ID NO: 82) c[c[Cys-AZE-His-D-Phe-His-Trp-Cys]- Ala-Lys-Lys-Lys-Trp] |
| 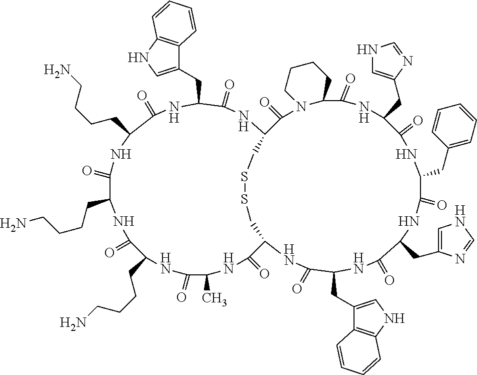 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N1CCCC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 83 (SEQ ID NO: 83) c[c[Cys-PIP-His-D-Phe-His-Trp-Cys]- Ala-Lys-Lys-Lys-Trp] |
| 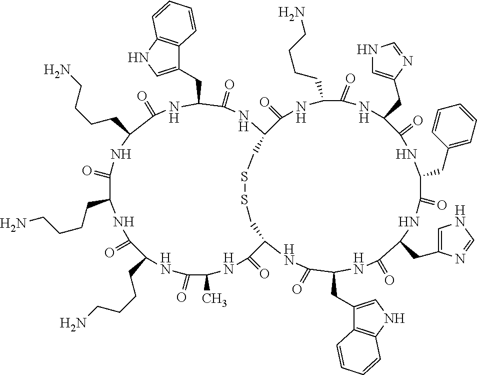 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N[C@H](CCCCN)C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 84 (SEQ ID NO: 84) c[c[Cys-D-Lys-His-D-Phe-His-Trp- Cys]-Ala-Lys-Lys-Lys-Trp] |
| 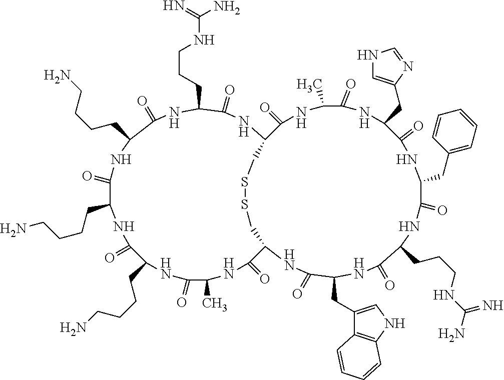 [SMILES: C[C@@H]1NC(=O)[C@@H]2CSSC[C@H](NC(=O)[C@H](CCCNC(=N)N)NC(=O)[C@H](CCCCN)NC(=O)[C@H](CCCCN)NC(=O)[C@H](CCCCN)NC1=O)C(=O)N[C@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 85 (SEQ ID NO: 85) c[c[Cys-D-Ala-His-D-Phe-Arg-Trp- Cys]-Ala-Lys-Lys-Lys-Arg] |
| 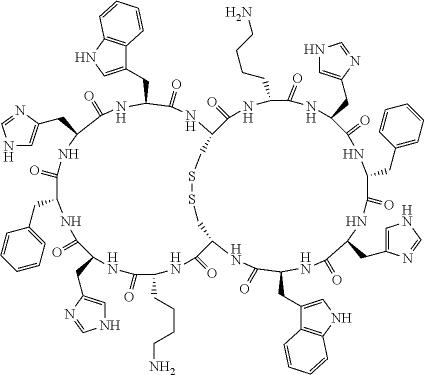 [SMILES: NCCCC[C@H]1NC(=O)[C@@H]2CSSC[C@H](NC(=O)[C@H](Cc3c[nH]c4ccccc34)NC(=O)[C@H](Cc3c[nH]cn3)NC(=O)[C@@H](Cc3ccccc3)NC(=O)[C@H](Cc3c[nH]cn3)NC1=O)C(=O)N[C@H](CCCCN)C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 86 (SEQ ID NO: 86) c[c[Cys-D-Lys-His-D-Phe-His-Trp- Cys]-D-Lys-His-D-Phe-His-Trp] |
| 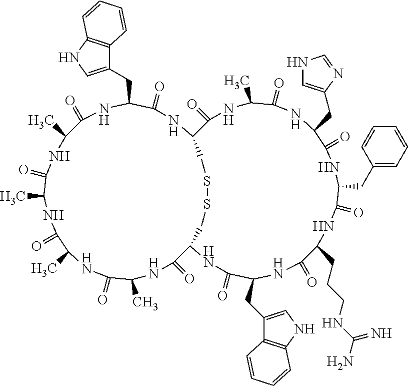 [SMILES: C[C@@H]1NC(=O)[C@H](C)NC(=O)[C@H](C)NC(=O)[C@@H]2CSSC[C@H](NC(=O)[C@H](Cc3c[nH]c4ccccc34)NC(=O)[C@H](C)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 87 (SEQ ID NO: 87) c[c[Cys-Ala-His-D-Phe-Arg-Trp-Cys]- Ala-Ala-Ala-Ala-Trp] |
| 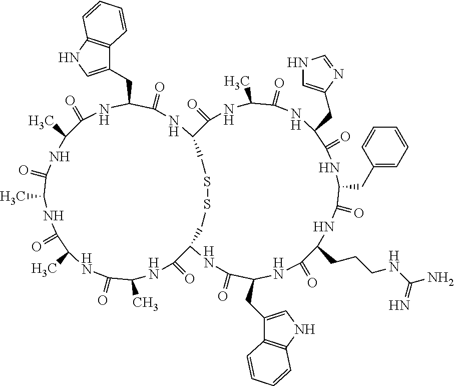 [SMILES: C[C@@H]1NC(=O)[C@H](C)NC(=O)[C@@H]2CSSC[C@H](NC(=O)[C@H](Cc3c[nH]c4ccccc34)NC(=O)[C@H](C)NC(=O)[C@@H](C)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 88 (SEQ ID NO: 88) c[c[Cys-Ala-His-D-Phe-Arg-Trp-Cys]- Ala-Ala-D-Ala-Ala-Trp] |
| 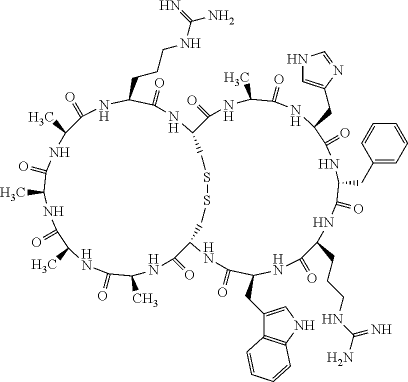 [SMILES: C[C@@H]1NC(=O)[C@H](C)NC(=O)[C@H](C)NC(=O)[C@@H]2CSSC[C@H](NC(=O)[C@H](CCCNC(=N)N)NC(=O)[C@H](C)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 89 (SEQ ID NO: 89) c[c[Cys-Ala-His-D-Phe-Arg-Trp-Cys]- Ala-Ala-Ala-Ala-Arg] |
| 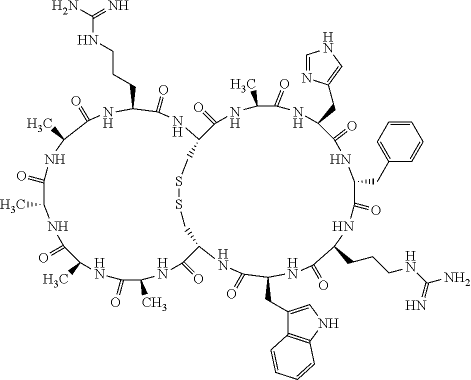 [SMILES: C[C@@H]1NC(=O)[C@H](C)NC(=O)[C@@H]2CSSC[C@H](NC(=O)[C@H](CCCNC(=N)N)NC(=O)[C@H](C)NC(=O)[C@@H](C)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 90 (SEQ ID NO: 90) c[c[Cys-Ala-His-D-Phe-Arg-Trp-Cys]- Ala-Ala-D-Ala-Ala-Arg] |
| 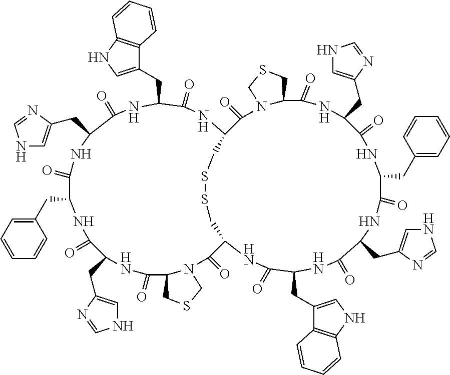 [SMILES: O=C1N[C@H]2CSSC[C@H](NC(=O)[C@H](Cc3c[nH]c4ccccc34)NC(=O)[C@H](Cc3c[nH]cn3)NC(=O)[C@@H](Cc3ccccc3)NC(=O)[C@H](Cc3c[nH]cn3)NC(=O)[C@@H]3CSCN3C2=O)C(=O)N2CSC[C@H]2C(=O)N[C@@H](Cc2c[nH]cn2)C(=O)N[C@H](Cc2ccccc2)C(=O)N[C@@H](Cc2c[nH]cn2)C(=O)N[C@H]1Cc1c[nH]c2ccccc12] | Chem. 91 (SEQ ID NO: 91) c[c[Cys-THAZ-His-D-Phe-His-Trp- Cys]-THAZ-His-D-Phe-His-Trp] |
| 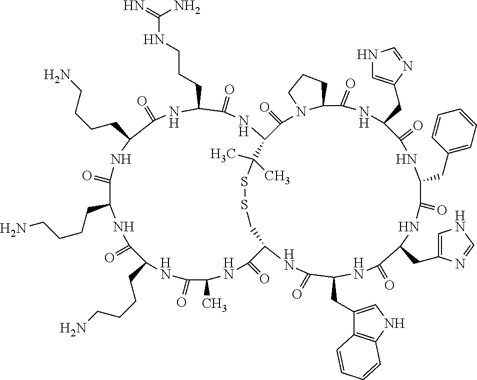 [SMILES: C[C@@H]1NC(=O)[C@@H]2CSSC(C)(C)[C@H](NC(=O)[C@H](CCCNC(=N)N)NC(=O)[C@H](CCCCN)NC(=O)[C@H](CCCCN)NC(=O)[C@H](CCCCN)NC1=O)C(=O)N1CCC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 92 (SEQ ID NO: 92) c[c[Pen-Pro-His-D-Phe-His-Trp-Cys]- Ala-Lys-Lys-Lys-Arg] |
| 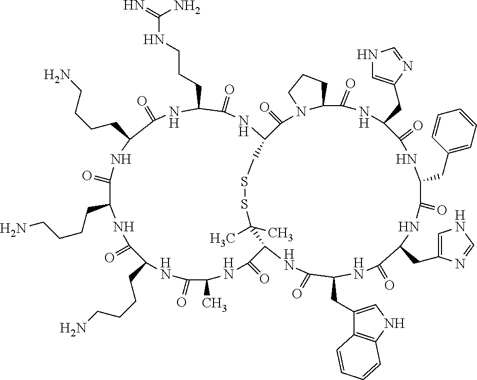 [SMILES: C[C@@H]1NC(=O)[C@H]2NC(=O)[C@H](Cc3c[nH]c4ccccc34)NC(=O)[C@H](Cc3c[nH]cn3)NC(=O)[C@@H](Cc3ccccc3)NC(=O)[C@H](Cc3c[nH]cn3)NC(=O)[C@@H]3CCCN3C(=O)[C@H](CSSC2(C)C)NC(=O)[C@H](CCCNC(=N)N)NC(=O)[C@H](CCCCN)NC(=O)[C@H](CCCCN)NC(=O)[C@H](CCCCN)NC1=O] | Chem. 93 (SEQ ID NO: 93) c[c[Cys-Pro-His-D-Phe-His-Trp-Pen]- Ala-Lys-Lys-Lys-Arg] |
| 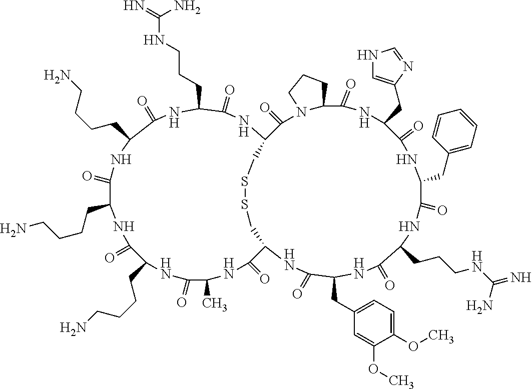 [SMILES: COc1ccc(C[C@@H]2NC(=O)[C@H](CCCNC(=N)N)NC(=O)[C@@H](Cc3ccccc3)NC(=O)[C@H](Cc3c[nH]cn3)NC(=O)[C@@H]3CCCN3C(=O)[C@@H]3CSSC[C@H](NC2=O)C(=O)N[C@@H](C)C(=O)N[C@@H](CCCCN)C(=O)N[C@@H](CCCCN)C(=O)N[C@@H](CCCCN)C(=O)N[C@@H](CCCNC(=N)N)C(=O)N3)cc1OC] | Chem. 94 (SEQ ID NO: 94) c[c[Cys-Pro-His-D-Phe-Arg-3,4- DiMeO-Phe-Cys]-Ala-Lys-Lys-Lys- Arg] |
| 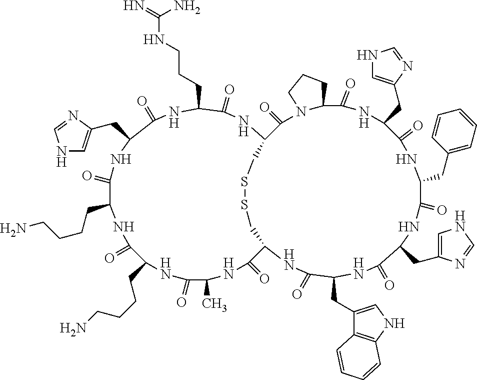 [SMILES: C[C@@H]1NC(=O)[C@@H]2CSSC[C@H](NC(=O)[C@H](CCCNC(=N)N)NC(=O)[C@H](Cc3c[nH]cn3)NC(=O)[C@H](CCCCN)NC(=O)[C@H](CCCCN)NC1=O)C(=O)N1CCC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 95 (SEQ ID NO: 95) c[c[Cys-Pro-His-D-Phe-His-Trp-Cys]- Ala-Lys-Lys-His-Arg] |
| 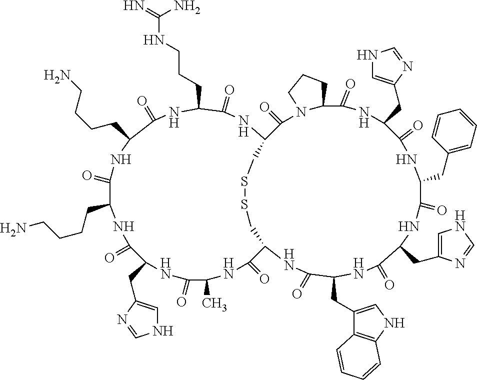 [SMILES: C[C@@H]1NC(=O)[C@@H]2CSSC[C@H](NC(=O)[C@H](CCCNC(=N)N)NC(=O)[C@H](CCCCN)NC(=O)[C@H](CCCCN)NC(=O)[C@H](Cc3c[nH]cn3)NC1=O)C(=O)N1CCC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 96 (SEQ ID NO: 96) c[c[Cys-Pro-His-D-Phe-His-Trp-Cys]- Ala-His-Lys-Lys-Arg] |
| 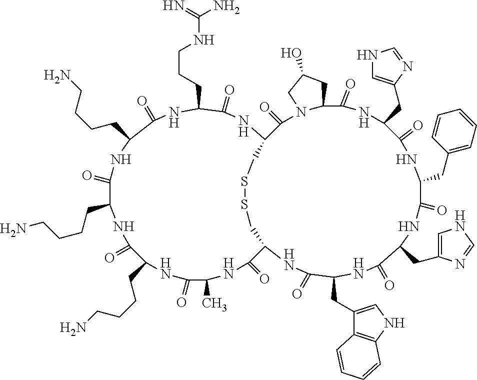 [SMILES: C[C@@H]1NC(=O)[C@@H]2CSSC[C@H](NC(=O)[C@H](CCCNC(=N)N)NC(=O)[C@H](CCCCN)NC(=O)[C@H](CCCCN)NC(=O)[C@H](CCCCN)NC1=O)C(=O)N1C[C@H](O)C[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 97 (SEQ ID NO: 97) c[c[Cys-Hyp-His-D-Phe-His-Trp-Cys]- Ala-Lys-Lys-Lys-Arg] |
| 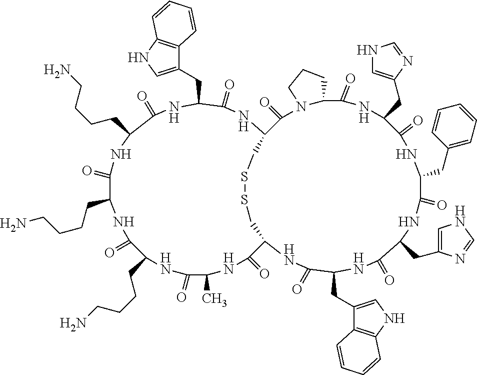 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N1CCC[C@@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 98 (SEQ ID NO: 98) c[c[Cys-D-Pro-His-D-Phe-His-Trp- Cys]-Ala-Lys-Lys-Lys-Trp] |
| 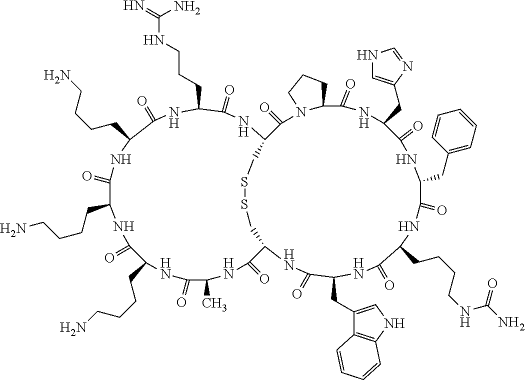 [SMILES: C[C@@H]1NC(=O)[C@@H]2CSSC[C@H](NC(=O)[C@H](CCCNC(=N)N)NC(=O)[C@H](CCCCN)NC(=O)[C@H](CCCCN)NC(=O)[C@H](CCCCN)NC1=O)C(=O)N1CCC[C@H]1C(=O)N[C@@H](Cc1c[nH]cn1)C(=O)N[C@H](Cc1ccccc1)C(=O)N[C@@H](CCCCNC(N)=O)C(=O)N[C@@H](Cc1c[nH]c3ccccc13)C(=O)N2] | Chem. 99 (SEQ ID NO: 99) c[c[Cys-Pro-His-D-Phe-Cit-Trp-Cys]- Ala-Lys-Lys-Lys-Arg] |
| 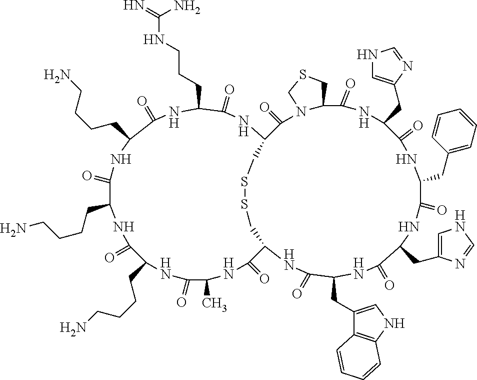 [SMILES: C[C@@H]1NC(=O)[C@@H]2CSSC[C@H](NC(=O)[C@H](CCCNC(=N)N)NC(=O)[C@H](CCCCN)NC(=O)[C@H](CCCCN)NC(=O)[C@H](CCCCN)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 100 (SEQ ID NO: 100) c[c[Cys-THAZ-His-D-Phe-His-Trp- Cys]-Ala-Lys-Lys-Lys-Arg] |
| 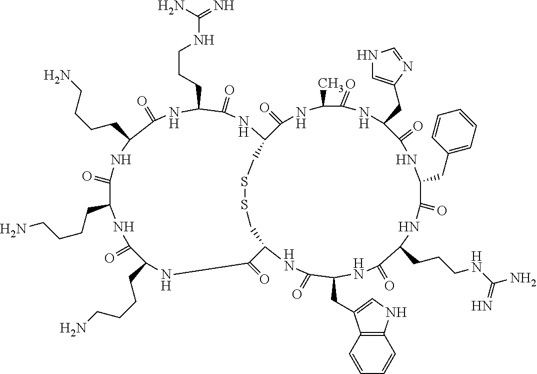 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](CCCCN)C(=O)N[C@@H](CCCCN)C(=O)N[C@@H](CCCCN)C(=O)N[C@@H](CCCNC(=N)N)C(=O)N2] | Chem. 101 (SEQ ID NO: 101) c[c[Cys-Ala-His-D-Phe-Arg-Trp-Cys]- Lys-Lys-Lys-Arg] |
| 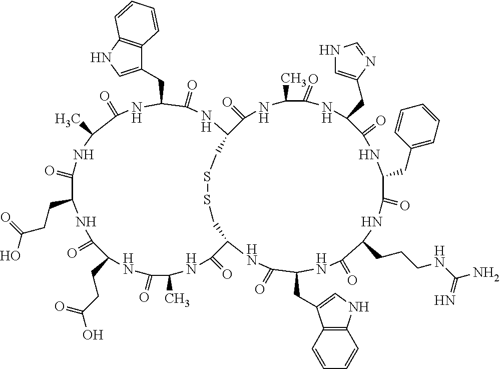 [SMILES: C[C@@H]1NC(=O)[C@H](CCC(=O)O)NC(=O)[C@H](CCC(=O)O)NC(=O)[C@H](C)NC(=O)[C@@H]2CSSC[C@H](NC(=O)[C@H](Cc3c[nH]c4ccccc34)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 102 (SEQ ID NO: 102) c[c[Cys-Ala-His-D-Phe-Arg-Trp-Cys]- Ala-Glu-Glu-Ala-Trp] |
| 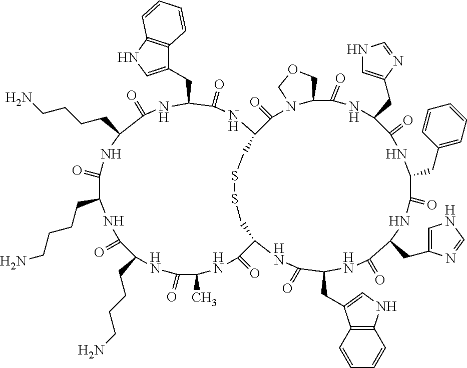 [SMILES: C[C@@H]1NC(=O)[C@@H]2CSSC[C@H](NC(=O)[C@H](Cc3c[nH]c4ccccc34)NC(=O)[C@H](CCCCN)NC(=O)[C@H](CCCCN)NC(=O)[C@H](CCCCN)NC1=O)C(=O)N1COC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 103 (SEQ ID NO: 103) c[c[Cys-OXA-His-D-Phe-His-Trp- Cys]-Ala-Lys-Lys-Lys-Trp] |
| 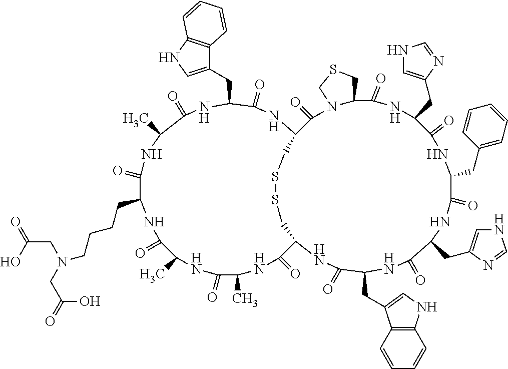 [SMILES: C[C@@H]1NC(=O)[C@H](C)NC(=O)[C@@H]2CSSC[C@H](NC(=O)[C@H](Cc3c[nH]c4ccccc34)NC(=O)[C@H](C)NC(=O)[C@H](CCCCN(CC(=O)O)CC(=O)O)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 104 (SEQ ID NO: 104) c[c[Cys-THAZ-His-D-Phe-His-Trp- Cys]-Ala-Ala-Lys(CH2COOH)₂-Ala- Trp] |
| 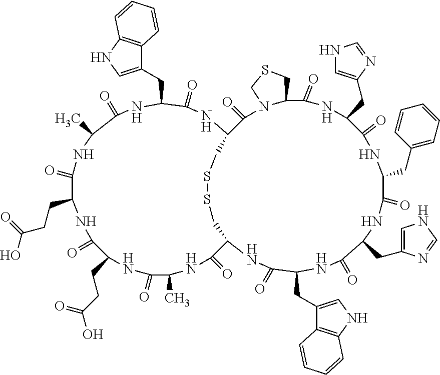 [SMILES: C[C@@H]1NC(=O)[C@H](CCC(=O)O)NC(=O)[C@H](CCC(=O)O)NC(=O)[C@H](C)NC(=O)[C@@H]2CSSC[C@H](NC(=O)[C@H](Cc3c[nH]c4ccccc34)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 105 (SEQ ID NO: 105) c[c[Cys-THAZ-His-D-Phe-His-Trp- Cys]-Ala-Glu-Glu-Ala-Trp] |
| 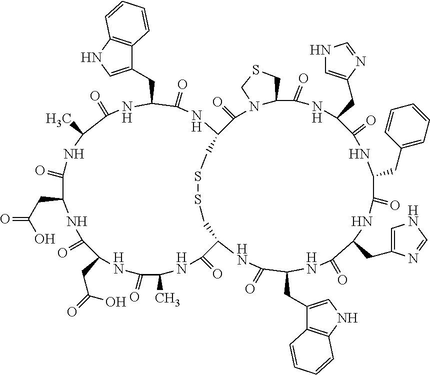 [SMILES: C[C@@H]1NC(=O)[C@H](CC(=O)O)NC(=O)[C@H](CC(=O)O)NC(=O)[C@H](C)NC(=O)[C@@H]2CSSC[C@H](NC(=O)[C@H](Cc3c[nH]c4ccccc34)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 106 (SEQ ID NO: 106) c[c[Cys-THAZ-His-D-Phe-His-Trp- Cys]-Ala-Asp-Asp-Ala-Trp] |
| 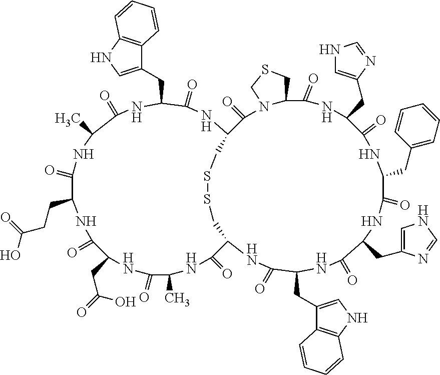 [SMILES: C[C@@H]1NC(=O)[C@H](CCC(=O)O)NC(=O)[C@H](CC(=O)O)NC(=O)[C@H](C)NC(=O)[C@@H]2CSSC[C@H](NC(=O)[C@H](Cc3c[nH]c4ccccc34)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 107 (SEQ ID NO: 107) c[c[Cys-THAZ-His-D-Phe-His-Trp- Cys]-Ala-Asp-Glu-Ala-Trp] |
| 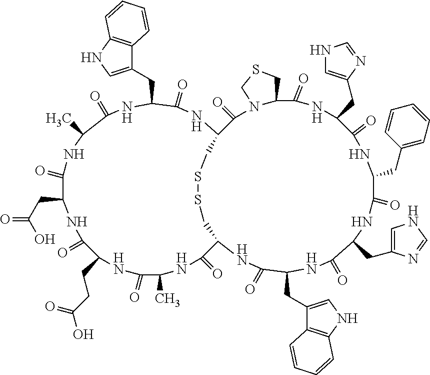 [SMILES: C[C@@H]1NC(=O)[C@H](CC(=O)O)NC(=O)[C@H](CCC(=O)O)NC(=O)[C@H](C)NC(=O)[C@@H]2CSSC[C@H](NC(=O)[C@H](Cc3c[nH]c4ccccc34)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 108 (SEQ ID NO: 108) c[c[Cys-THAZ-His-D-Phe-His-Trp- Cys]-Ala-Glu-Asp-Ala-Trp] |
| 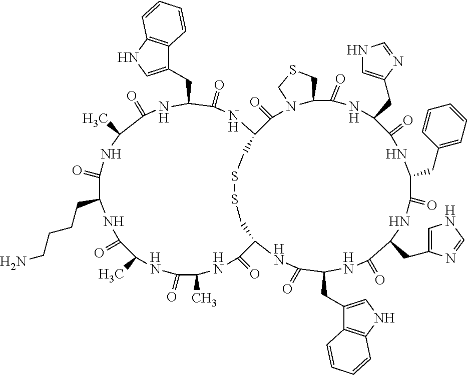 [SMILES: C[C@@H]1NC(=O)[C@H](C)NC(=O)[C@@H]2CSSC[C@H](NC(=O)[C@H](Cc3c[nH]c4ccccc34)NC(=O)[C@H](C)NC(=O)[C@H](CCCCN)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 109 (SEQ ID NO: 109) c[c[Cys-THAZ-His-D-Phe-His-Trp- Cys]-Ala-Ala-Lys-Ala-Trp] |
| 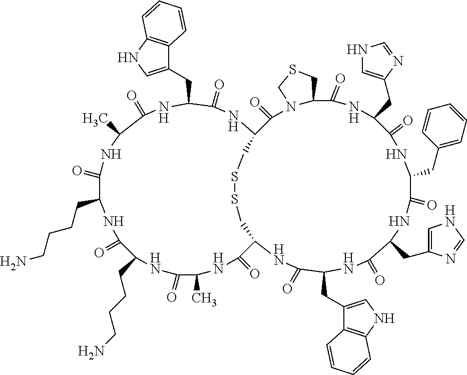 [SMILES: C[C@@H]1NC(=O)[C@H](CCCCN)NC(=O)[C@H](CCCCN)NC(=O)[C@H](C)NC(=O)[C@@H]2CSSC[C@H](NC(=O)[C@H](Cc3c[nH]c4ccccc34)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 110 (SEQ ID NO: 110) c[c[Cys-THAZ-His-D-Phe-His-Trp- Cys]-Ala-Lys-Lys-Ala-Trp] |
| 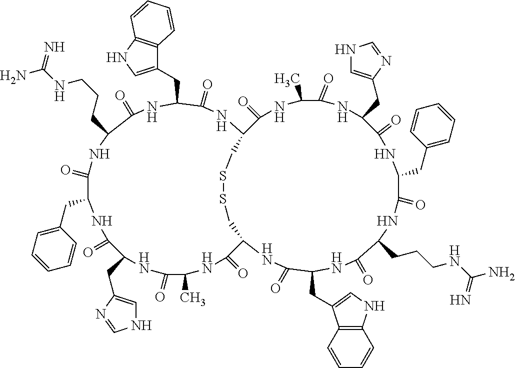 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N[C@@H](C)C(=O)N[C@@H](Cc1c[nH]cn1)C(=O)N[C@H](Cc1ccccc1)C(=O)N[C@@H](CCCNC(=N)N)C(=O)N[C@@H](Cc1c[nH]c3ccccc13)C(=O)N2] | Chem. 111 (SEQ ID NO: 111) c[c[Cys-Ala-His-D-Phe-Arg-Trp-Cys]- Ala-His-D-Phe-Arg-Trp] |
| 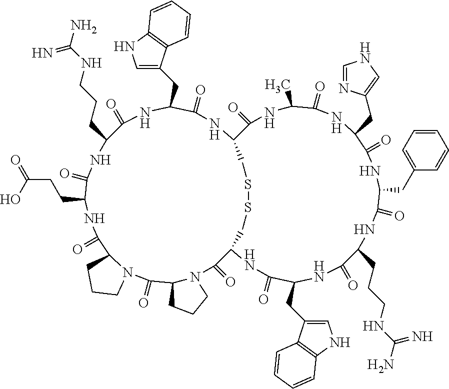 [SMILES: C[C@@H]1NC(=O)[C@@H]2CSSC[C@H](NC(=O)[C@H](Cc3c[nH]c4ccccc34)NC(=O)[C@H](CCCNC(=N)N)NC(=O)[C@@H](Cc3ccccc3)NC(=O)[C@H](Cc3c[nH]cn3)NC1=O)C(=O)N1CCC[C@H]1C(=O)N1CCC[C@H]1C(=O)N[C@@H](CCC(=O)O)C(=O)N[C@@H](CCCNC(=N)N)C(=O)N[C@@H](Cc1c[nH]c3ccccc13)C(=O)N2] | Chem. 112 (SEQ ID NO: 112) c[c[Cys-Ala-His-D-Phe-Arg-Trp-Cys]- Pro-Pro-Glu-Arg-Trp] |
| 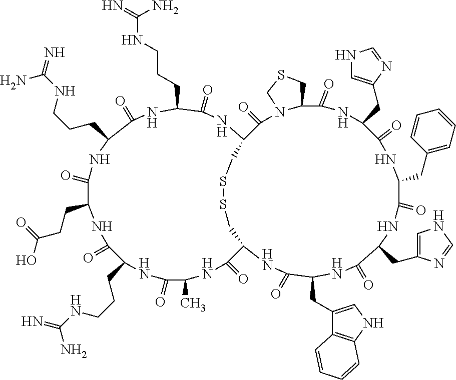 [SMILES: C[C@@H]1NC(=O)[C@@H]2CSSC[C@H](NC(=O)[C@H](CCCNC(=N)N)NC(=O)[C@H](CCCNC(=N)N)NC(=O)[C@H](CCC(=O)O)NC(=O)[C@H](CCCNC(=N)N)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 113 (SEQ ID NO: 113) c[c[Cys-THAZ-His-D-Phe-His-Trp- Cys]-Ala-Arg-Glu-Arg-Arg] |
| 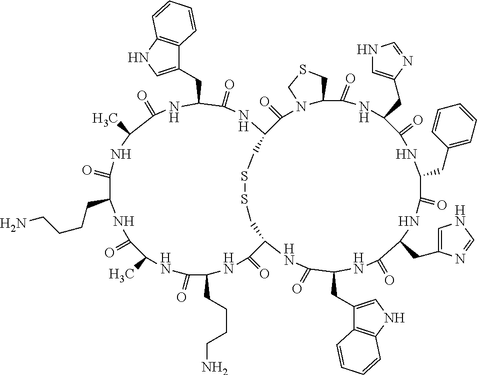 [SMILES: C[C@@H]1NC(=O)[C@H](CCCCN)NC(=O)[C@H](C)NC(=O)[C@H](CCCCN)NC(=O)[C@@H]2CSSC[C@H](NC(=O)[C@H](Cc3c[nH]c4ccccc34)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 114 (SEQ ID NO: 114) c[c[Cys-THAZ-His-D-Phe-His-Trp- Cys]-Lys-Ala-D-Lys-Ala-Trp] |
| 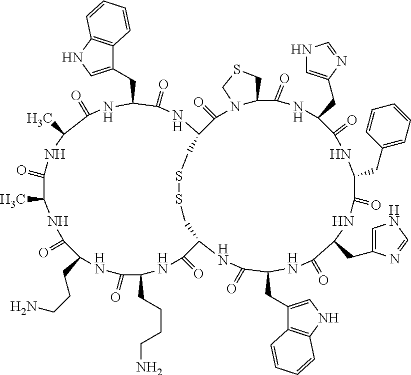 [SMILES: C[C@@H]1NC(=O)[C@H](C)NC(=O)[C@H](CCCN)NC(=O)[C@H](CCCCN)NC(=O)[C@@H]2CSSC[C@H](NC(=O)[C@H](Cc3c[nH]c4ccccc34)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 115 (SEQ ID NO: 115) c[c[Cys-THAZ-His-D-Phe-His-Trp-C ys]-Lys-Orn-Ala-Ala-Trp] |
| 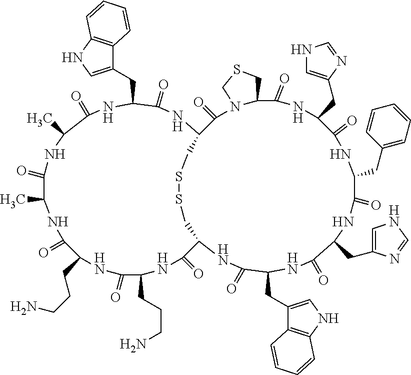 [SMILES: C[C@@H]1NC(=O)[C@H](C)NC(=O)[C@H](CCCN)NC(=O)[C@H](CCCN)NC(=O)[C@@H]2CSSC[C@H](NC(=O)[C@H](Cc3c[nH]c4ccccc34)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 116 (SEQ ID NO: 116) c[c[Cys-THAZ-His-D-Phe-His-Trp- Cys]-Orn-Orn-Ala-Ala-Trp] |
| 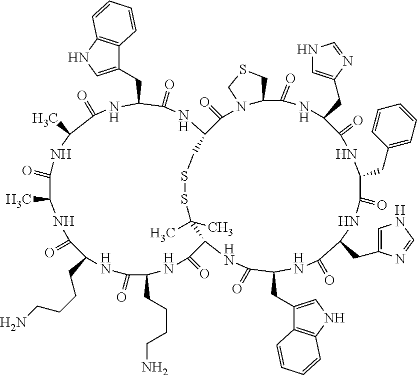 [SMILES: C[C@@H]1NC(=O)[C@H](C)NC(=O)[C@H](CCCCN)NC(=O)[C@H](CCCCN)NC(=O)[C@H]2NC(=O)[C@H](Cc3c[nH]c4ccccc34)NC(=O)[C@H](Cc3c[nH]cn3)NC(=O)[C@@H](Cc3ccccc3)NC(=O)[C@H](Cc3c[nH]cn3)NC(=O)[C@@H]3CSCN3C(=O)[C@H](CSSC2(C)C)NC(=O)[C@H](Cc2c[nH]c3ccccc23)NC1=O] | Chem. 117 (SEQ ID NO: 117) c[c[Cys-THAZ-His-D-Phe-His-Trp- Pen]-Lys-Lys-Ala-Ala-Trp] |
| 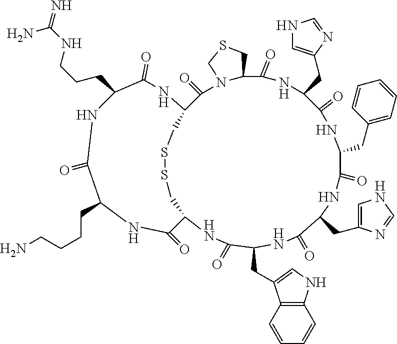 [SMILES: N=C(N)NCCC[C@@H]1NC(=O)[C@H](CCCCN)NC(=O)[C@@H]2CSSC[C@H](NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 118 (SEQ ID NO: 118) c[c[Cys-THAZ-His-D-Phe-His-Trp- Cys]-Lys-Arg] |
| 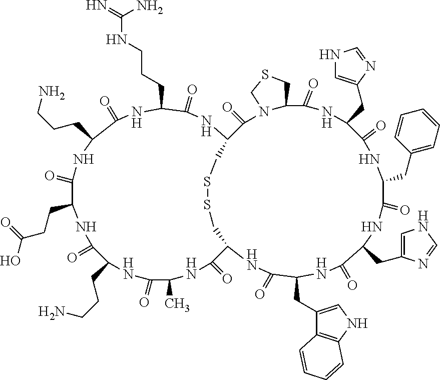 [SMILES: C[C@@H]1NC(=O)[C@@H]2CSSC[C@H](NC(=O)[C@H](CCCNC(=N)N)NC(=O)[C@H](CCCN)NC(=O)[C@H](CCC(=O)O)NC(=O)[C@H](CCCN)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 119 (SEQ ID NO: 119) c[c[Cys-THAZ-His-D-Phe-His-Trp- Cys]-Ala-Orn-Glu-Orn-Arg] |
| 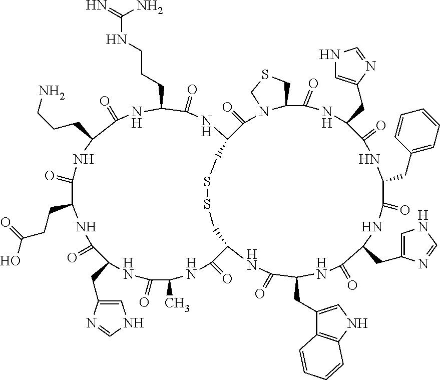 [SMILES: C[C@@H]1NC(=O)[C@@H]2CSSC[C@H](NC(=O)[C@H](CCCNC(=N)N)NC(=O)[C@H](CCCN)NC(=O)[C@H](CCC(=O)O)NC(=O)[C@H](Cc3c[nH]cn3)NC1=O)C(=O)N1CSC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 120 (SEQ ID NO: 120) c[c[Cys-THAZ-His-D-Phe-His-Trp- Cys]-Ala-His-Glu-Orn-Arg] |
| 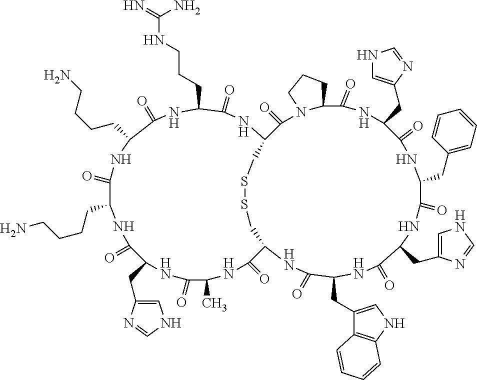 [SMILES: C[C@@H]1NC(=O)[C@@H]2CSSC[C@H](NC(=O)[C@H](CCCNC(=N)N)NC(=O)[C@@H](CCCCN)NC(=O)[C@@H](CCCCN)NC(=O)[C@H](Cc3c[nH]cn3)NC1=O)C(=O)N1CCC[C@H]1C(=O)N[C@@H](Cc1c[nH]cn1)C(=O)N[C@H](Cc1ccccc1)C(=O)N[C@@H](Cc1c[nH]cn1)C(=O)N[C@@H](Cc1c[nH]c3ccccc13)C(=O)N2] | Chem. 121 (SEQ ID NO: 121) c[c[Cys-Pro-His-D-Phe-His-Trp-Cys]- Ala-His-D-Lys-D-Lys-Arg] |

22. The bicyclic compound according to any one of embodiments 1-21, wherein said compound is a compound according to SEQ ID NO:114 (chem. 114) optionally comprising 1, 2 or 3 amino acid substitutions.
23. The bicyclic compound according to any one of embodiments 1-21, wherein said compound is a compound according to SEQ ID NO:120 (chem. 120) optionally comprising 1, 2 or 3 amino acid substitutions.
24. The bicyclic compound according to embodiment 22 or 23 wherein said substitution is a conservative substitution.
25. The bicyclic compound according to any one of embodiments 22-24, wherein said compound comprises 1, 2 or 3 amino acid deletions and/or 1, 2 or 3 amino acid insertions.
26. The bicyclic compound according to any one of embodiments 1-25 wherein said compound is a MC4R agonist having a MC4R binding Ki of less than 100, less than 90, less than 80, less than 70, less than 60, less than 50, less than 40, less than 30, less than 20 or less than 10 nM as measured in a human MC4R competitive binding assay as described herein.
27. The bicyclic compound according to any one of embodiments 1-25 wherein said compound is capable of reducing food intake by about 5, 10, 15, 20, 25, 30, 35, 40, 45, 50, 55, 60, 65, 70 or 75% as determined in an in vivo obese model as described herein.
28. The bicyclic compound according to any one of embodiments 1-25 wherein said compound is capable of reducing food intake by 5-75%, 10-75%, 15-75%, 20-75%, 25-75%, 30-75%, 5-50%, 10-50%, 15-50%, 20-50%, 25-50%, 30-50%, 15-40% or 20-40%.
29. The bicyclic compound according to any one of embodiments 1-25 wherein said compound is capable of reducing body weight by 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% as determined using an in vivo obese model as described herein.
30. The bicyclic compound according to embodiment 29 wherein said compound is capable of reducing body weight by 1-30%.
31. The bicyclic compound according to any one of embodiments 1-21, wherein said compound is a compound according to SEQ ID NO:114 (chem. 114) optionally comprising 1, 2 or 3 amino acid substitutions.
32. The bicyclic compound according to any one of embodiments 1-21, wherein said compound is a compound according to SEQ ID NO:120 (chem. 120) optionally comprising 1, 2 or 3 amino acid substitutions.
33. The bicyclic compound according to embodiment 22 or 23 wherein said substitution is a conservative substitution.
34. The bicyclic compound according to any one of embodiments 22-24, wherein said compound comprises 1, 2 or 3 amino acid deletions and/or 1, 2 or 3 amino acid insertions.
35. The bicyclic compound according to any one of embodiments 1-25 wherein said compound is a MC4R agonist having a MC4R binding Ki of less than 100, less than 90, less than 80, less than 70, less than 60, less than 50, less than 40, less than 30, less than 20 or less than 10 nM as measured in a human MC4R competitive binding assay as described herein.
36. The bicyclic compound according to any one of embodiments 1-25 wherein said compound is capable of reducing food intake by about 5, 10, 15, 20, 25, 30, 35, 40, 45, 50, 55, 60, 65, 70 or 75% as determined in an in vivo obese model as described herein.
37. The bicyclic compound according to any one of embodiments 1-25 wherein said compound is capable of reducing food intake by 5-75%, 10-75%, 15-75%, 20-75%, 25-75%, 30-75%, 5-50%, 10-50%, 15-50%, 20-50%, 25-50%, 30-50%, 15-40% or 20-40%.
38. The bicyclic compound according to any one of embodiments 1-25 wherein said compound is capable of reducing body weight by 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% as determined using an in vivo obese model as described herein.
39. The bicyclic compound according to embodiment 29 wherein said compound is capable of reducing body weight by 1-30%.
40. The bicyclic compound according to embodiment 30 wherein said compound is capable of reducing body weight by 15-30%.
41. A method of reducing food intake wherein a compound according to any one of embodiments 1-25 is administered to a patient in need thereof.
42. The method according to embodiment 32 wherein said compound is capable of reducing food intake by 5, 10, 15, 20, 25, 30, 35, 40, 45, 50, 55, 60, 65, 70 or 75% as determined in an in vivo obese model as described herein.
43. The method according to embodiment 32 wherein said compound is capable of reducing food intake by 5-75%, 10-75%, 15-75%. 20-75%, 25-75%, 30-75%, 5-50%, 10-50%, 15-50%. 20-50%, 25-50%, 30-50%, 15-40% or 20-40%.
44. A method of reducing body weight wherein a compound according to any one of embodiments 1-25 is administrated to a patient in need thereof.
45. The method according to embodiment 35 wherein body weight is reduced by 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% as determined using an in vivo obese model as described herein.
46. The method according to embodiment 35 wherein body weight is reduced by 1-30%.
47. The method according to embodiment 37 wherein body weight is reduced by 15-30%.
48. A method of treating obesity or overweight comprising administering to a patient in need thereof an effective amount of a compound according to any one of embodiments 1-25.
49. A method of treating, in an obese patient, a disease or state selected from atherosclerosis, hypertension, type 2 diabetes, IGT, dyslipidemia, coronary heart disease, gallbladder disease, gall stone, osteoarthritis, cancer, sexual dysfunction, hypothalamic amenorrhea, risk of premature death, neuronal protection, effect in ischemic heart disease or anti-inflammatory effects, comprising administering to an obese patient in need thereof an effective amount of a compound according to any one of embodiments 1-25.
50. A method of treating a disease or state selected from Nonalcoholic Fatty Liver Disease (NAFLD) and Nonalcoholic Steatohepatitis (NASH) comprising administering an effective amount of a compound according to any one of embodiments 1-25.
51. The compound according to any one of embodiments 1-25 for use in medicine.
52. The compound according to any one of embodiments 1-25 for use in the treatment of obesity or overweight.
53. The compound according to any one of embodiments 1-25 for use in the treatment of a disease or state related to overweight or obesity; treating bulimia; treating binge-eating; treating atherosclerosis, hypertension, type 2 diabetes, IGT, dyslipidemia, coronary heart disease, gallbladder disease, gall stone, osteoarthritis, cancer, sexual dysfunction, hypothalamic amenorrhea or risk of premature death; or treating, in an obese patient, a disease or state selected from type 2 diabetes, IGT, coronary heart disease, gallbladder disease, gall stone, osteoarthritis, cancer, sexual dysfunction, risk of premature death; for providing neuronal protection, for having an effect on ischemic heart disease, cerebral ischemia or anti-inflammatory effects and for the treatment of autoimmune diseases, e.g. multiple sclerosis.
54. The compound according to any one of embodiments 1-25 for use in the treatment of Nonalcoholic Fatty Liver Disease (NAFLD) and Nonalcoholic Steatohepatitis (NASH).
55. Use of a compound according to any one of embodiments 1-25 for the manufacture of a medicament for use in the treatment of obesity or overweight.
56. Use of a compound according to any one of embodiments 1-25 for the manufacture of a medicament for use in the treatment of a disease or state related to overweight or obesity; the treatment of bulimia; the treatment of binge-eating; the treatment of atherosclerosis, hypertension, type 2 diabetes, IGT, dyslipidemia, coronary heart disease, gallbladder disease, gall stone, osteoarthritis, cancer, sexual dysfunction, hypothalamic amenorrhea or risk of premature death; or treating, in an obese patient, a disease or state selected from type 2 diabetes, IGT, coronary heart disease, gallbladder disease, gall stone, osteoarthritis, cancer, sexual dysfunction, risk of premature death; for providing neuronal protection, for having an effect on ischemic heart disease, cerebral ischemia or anti-inflammatory effects and for the treatment of autoimmune diseases, e.g. multiple sclerosis.
57. Use of a compound according to any one of embodiments 1-25 for the manufacture of a medicament for use in the treatment of Nonalcoholic Fatty Liver Disease (NAFLD) and Nonalcoholic Steatohepatitis (NASH).
58. A pharmaceutical composition comprising a compound according to any one of embodiments 1-25.
59. The pharmaceutical composition according to embodiment 49, further comprising one or more additional therapeutically active compounds or substances.
60. The pharmaceutical composition according to embodiment 50 wherein said additional therapeutically active compound or substance is selected from anti-diabetic agents, anti-hyperlipidemic agents, anti-obesity agents, anti-hypertensive agents and agents for the treatment of complications resulting from, or associated with, diabetes.
61. The pharmaceutical composition according to any one of embodiments 49-51, wherein the pH is neutral to weakly basic.
62. The pharmaceutical composition according to any one of embodiments 49-51, wherein the pH is neutral to weakly acidic.
63. The pharmaceutical composition according to any one of embodiments 49-51, wherein the pH is from about 7.0 to about 8.0.
64. The pharmaceutical composition according to any one of embodiments 49-51, wherein the pH is about 6.0 to about 7.0.
65. The pharmaceutical composition according to embodiment 55, wherein the pH is about 6.
66. The pharmaceutical composition according to any one of embodiments 49-56, wherein said composition is in a unit dosage form comprising from about 0.05 mg to about 1000 mg of said compound.
67. The method, use, compound or composition according to any one of the previous embodiments, wherein said compound or composition is administered once daily.
68. The method, use, compound or composition according to any one of the previous embodiments, wherein said compound or composition is administered once every second day, every third day, every fourth day or every fifth day.
69. The method, use, compound or composition according to any one of the previous embodiments, wherein said compound or composition is administered once weekly.
70. The method, use, compound or composition according to any one of the previous embodiments, wherein said compound or composition is administered parenterally, orally, nasally, buccally or sublingually.
71. The method, use, compound or composition according to embodiment 61, wherein said compound or composition is administered parenterally.
72. A method of activating MC4R in a subject, the method comprising administering to said subject an effective amount of a compound according to any one of embodiments 1-25.

Further Non-Limiting Embodiments are Listed Below:

In one embodiment the compounds as disclosed herein are not linked to a half-life extending (protracting) moiety.

In one embodiment the compounds as disclosed herein are highly selective for the MC4R as compared to the MC1R.

In one such embodiment the compounds as disclosed herein exhibit an affinity for MC4R which is 50-, 100-, 200-, 300-, 400-, 500-, 600-, 700-, 800-, 900-, 1000-, 2000-, 3000-, 4000-, 5000-, 6000-, 7000-, 8000-, 9000- or 10000-fold (or more) higher than that for MC1R (based on Ki (nM)).

In one embodiment the compounds as disclosed herein are highly potent in relation to MC4R activation.

In one embodiment the compounds as disclosed herein are highly potent in relation to MC4R activation and significantly less potent in relation to MC1R activation.

In one embodiment the compounds as disclosed herein exhibit a significantly lower EC₅₀ value in relation to MC4R activation as compared to MC1R activation (based on cAMP signalling (nM)).

In some embodiments the compounds as disclosed herein exhibit an EC₅₀ in relation to MC4R activation which is 5-, 10-, 20-, 30-, 40-, 50-, 60-, 70-, 80-, 90-, 100-, 150-, 200-, 250-, 300-, 350-, 400-, 450-, 500-, 600-, 700-, 800-, 900- or 1000-fold (or more) lower than the EC₅₀ in relation to MC1R activation (based on cAMP signalling (nM) as as described in Example 3 herein).

In one embodiment the compounds as disclosed herein exhibit a MC1R/MC4R EC₅₀ ratio is larger than 20, 50, 100, 200, 300, 400, 500, 600, 700, 800, 900 or 1000.

In one embodiment a compound of the invention is a bicyclic MC4R agonist comprising a backbone wherein said backbone comprises a sequence represented by the following amino acid residues Cys-X-His-D-Phe-His-Trp-Cys, wherein X is Pro or THAZ. In one such embodiment the bicyclic compound is has a length of 8, 9, 10, 11, 12, 13, 14 or 15 amino acid residues. In one such embodiment the compound comprises a disulphide bond or methylene bridge. In one such embodiment the bicyclic compound is suitable for use in the treatment of obesity or overweight. The present invention also encompasses combinations of two or more embodiments of compounds of the invention as outlined above.

Sequences listed in the accompanying (electronic) sequence listing are all bicyclic compounds with the exception of SEQ ID NO:7 (Chem. 7).

### EXAMPLES

### Abbreviations

Aib: 2-amino isobutyric acid

### DCM: Dichloromethane

### DIC: Diisopropylcarbodiimide

DIO: Diet-induced obese

### DMF: Dimethylformamide

### LCMS: Liquid Chromatography Mass Spectrometry

### MALDI-MS: Matrix-assisted Laser Desorption/Ionization Mass Spectrometry

MC4R: Melanocortin 4 receptor
NMP: N-methyl pyrrolidin-2-one

### RP-HPLC: Reversed Phase High Performance Liquid Chromatography

### RT: Room Temperature

### SPPS: Solid Phase Peptide Synthesis

OXYMA PURE: Cyano-hydroxyimino-acetic acid ethyl ester
TFA: Trifluoroacetic acid
TIS: Triisopropyl silane

### UPLC: Ultra Performance Liquid Chromatography

### Example 1: General Methods of Preparation

Methods of Preparation

The compounds of the invention may be prepared as is known in the art.

In one aspect the pharmaceutical compounds and formulations may be prepared as described in the examples herein.

The production of peptides, e.g., bicyclic peptides, as disclosed herein is well known in the art. The bicyclic peptide may for instance be produced by classical peptide synthesis, e.g., solid phase peptide synthesis using Boc or Fmoc chemistry or other well established techniques, see, e.g., Greene and Wuts, “Protective Groups in Organic Synthesis”, John Wiley & Sons, 1999, Florencio Zaragoza Dorwald, “Organic Synthesis on solid Phase”, Wiley-VCH Verlag GmbH, 2000, and “Fmoc Solid Phase Peptide Synthesis”, Edited by W. C. Chan and P. D. White, Oxford University Press, 2000.

Method of the Introduction of a Methylene Bridge into a Disulfide Bridge

A methylene bridge or thioacetal may be introduced as described in Kourra C. M. B. K., Cramer N. Converting disulfide bridges in native peptides to stable methylene thioacetals. Chem Sci. 2016; 7(12):7007-7012.

General Methods of Preparation Using Solid Phase Peptide Synthesis

This section relates to methods for solid phase peptide synthesis (SPPS methods, including methods for de-protection of amino acids, methods for cleaving the peptide from the resin, and for its purification), as well as methods for detecting and characterising the resulting peptide (LCMS, MALDI-MS, and UPLC methods). The solid phase synthesis of peptides may in some cases be improved by the use of di-peptides protected on the di-peptide amide bond with a group that can be cleaved under acidic conditions such as, but not limited to, 2-Fmoc-oxy-4-methoxybenzyl, or 2,4,6-trimethoxybenzyl. In cases where a serine or a threonine is present in the peptide, pseudoproline di-peptides may be used (available from, e.g., Novabiochem, see also W. R. Sampson (1999), J. Pep. Sci. 5, 403). The Fmoc-protected amino acid derivatives used were the standard recommended: Fmoc-Ala-OH, Fmoc-Arg(Pbf)-OH, Fmoc-Asn(Trt)-OH, Fmoc-Asp(OtBu)—OH, Fmoc-Cys(Trt)-OH, Fmoc-Gln(Trt)-OH, Fmoc-Glu(OtBu)—OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)—OH, Fmoc-Thr(tBu)—OH, Fmoc-Trp(Boc)-OH, Fmoc-Tyr(tBu)—OH, or, Fmoc-Val-OH etc. supplied from e.g. Anaspec, Bachem, Iris Biotech, or Novabiochem. Where nothing else is specified the natural L-configuration of the amino acids are used.

Synthesis of Resin Bound Protected Peptide Hydrazides

Preparation of Hydrazine-Resin

2-Chlorotrityl chloride resin (25 g, 1.70 mmol/g) was swollen in DMF (125 ml) for 30 min under nitrogen, and then cooled to 0° C. A mixture of hydrazine.hydrate (7.7 ml, 160 mmol) and triethylamine (6.7 ml, 48 mmol) in DMF (10 ml) was added dropwise and the suspension was stirred 30 min at 25° C. The suspension was then cooled to 0° C. followed by dropwise addition of a mixture of hydrazine.hydrate (7.7 ml, 160 mmol) and triethylamine (6.7 ml, 48 mmol) in DMF (10 ml). The suspension was stirred 30 min at 25° C. and then filtered and washed with NMP (6×), water (6×), NMP (6×), methanol (6×) and DCM (6×). The resin was then dried in vacuo overnight to afford 22.60 g light-yellow resin.

Method: SPPS

SPPS was performed on a Prelude Solid Phase Peptide Synthesizer from Protein Technologies (Tucson, Ariz. 85714 U.S.A.) at 250-μmol or 400-μmol scale using six fold excess of Fmoc-amino acids (300 mM in NMP with 300 mM OXYMA PURE®) relative to resin loading (typical loading of hydrazine-resin was 0.3 mmol/g). Fmoc-deprotection was performed using 20% piperidine in NMP. Coupling was performed using 3:3:3:4 amino acid/OXYMA PURE®/DIC/collidine in NMP. NMP and DCM top washes (7 ml, 0.5 min, 2×2 each) were performed between deprotection and coupling steps. Coupling times were generally 60 minutes. Some amino acids including, but not limited to Fmoc-Arg(Pbf)-OH, Fmoc-Aib-OH, Fmoc-Cys(Trt)-OH or Boc-His(Trt)-OH were “double coupled”, meaning that after the first coupling (e.g. 60 min), the resin was drained and more reagents were added (amino acid, OXYMA PURE®, DIC, and collidine), and the mixture allowed to react again (e.g. 60 min).

Cleavage of Resin Bound Peptide Hydrazides and Purification

After synthesis the resin was washed with DCM, and the peptide was cleaved from the resin by a 3 hour treatment with TFA/TIS/2-mercaptoethanol/water (87.5/5/5/2.5) followed by precipitation with diethylether. The peptide was dissolved in a suitable solvent (such as e.g., 10/90 acetic acid/water) and purified by standard RP-HPLC on a C18, 5 μM column, using acetonitrile/water/TFA. The fractions were analysed by a combination of UPLC, MALDI-MS and LCMS methods, and the appropriate fractions were pooled.

Cyclization of Peptide Hydrazides, Disulfide Oxidation and Purification

Method: NCL_M1

The pooled fractions from RP-HPLC purification of the peptide hydrazides were diluted with water to 80:20 water/acetonitrile. Disodium phosphate was added to a final concentration of 0.2 M and the pH was adjusted to 3.0 with concentrated hydrochloric acid (aq). The mixture was cooled to 0° C. and sodium nitrite (10 eq, 0.2 M in water) was added, and the mixture was stirred for 20 minutes at 0° C. Sodium 2-mercaptoethanesulfonate (20 eq) was added and the pH was adjusted to 7.0 with 1 M NaOH (aq). The reaction mixture was stirred at 25° C. for 16 hours and then purified by standard RP-HPLC on a C18, 5 μM column, using acetonitrile/water/TFA. The fractions were analysed by a combination of UPLC, MALDI-MS and LCMS methods, and the appropriate fractions were pooled and lyophilized.

General Methods of Detection and Characterisation

LCMS was performed on a setup consisting of Waters Acquity UPLC system and LCT Premier XE mass spectrometer from Micromass. Eluents: A: 0.1% Formic acid in water, B: 0.1% Formic acid in acetonitrile.

The analysis was performed at room temperature (RT) by injecting an appropriate volume of the sample (preferably 2-10 μl) onto the column which was eluted with a gradient of A and B. The UPLC conditions, detector settings and mass spectrometer settings were:

Column: Waters Acquity UPLC BEH, C-18, 1.7 μm, 2.1 mm×50 mm.

Gradient: Linear 5%-95% acetonitrile during 4.0 min at 0.4 ml/min.

Detection: 214 nm (analogue output from TUV (Tunable UV detector))

MS ionisation mode: API-ES

Scan: 100-2000 amu (alternatively 500-2000 amu), step 0.1 amu.

The reverse phase-analysis was performed using a Waters UPLC system fitted with a dual band detector. UV detections at 214 nm and 254 nm were collected using an ACQUITY UPLC BEH, C18, 1.7 μm, 2.1 mm×150 mm column, 40° C. The UPLC system was connected to two eluent reservoirs containing: A: 99.95% H₂O, 0.05% TFA; B: 99.95% CH₃CN, 0.05% TFA. The following linear gradient was used: 95% A, 5% B to 5% A, 95% B over 16 minutes at a flow-rate of 0.40 ml/min.

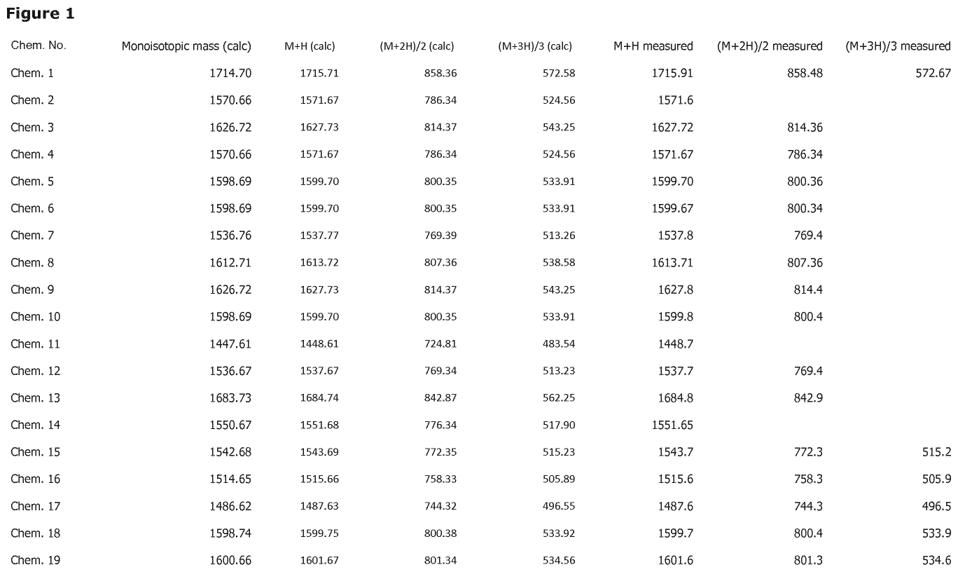

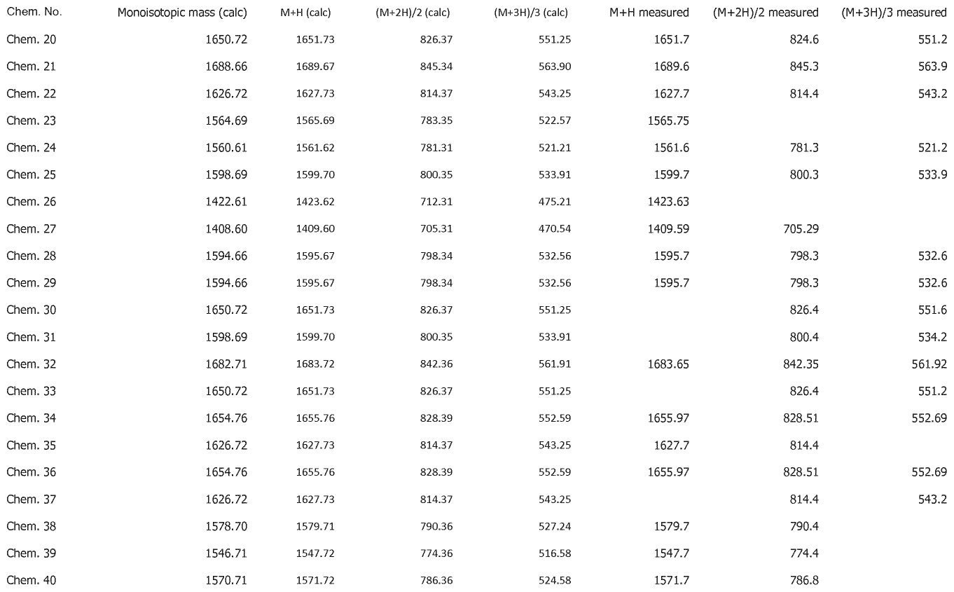

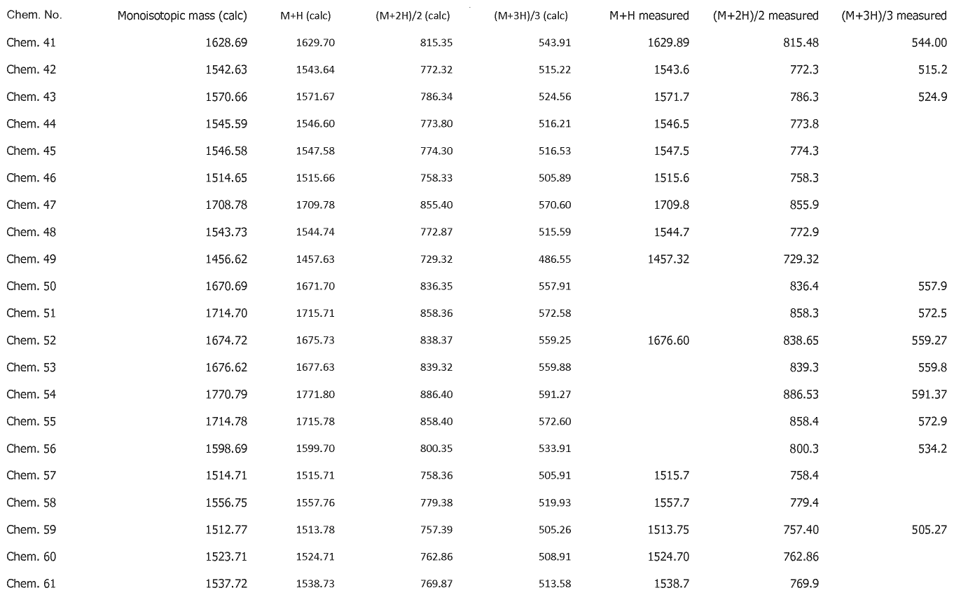

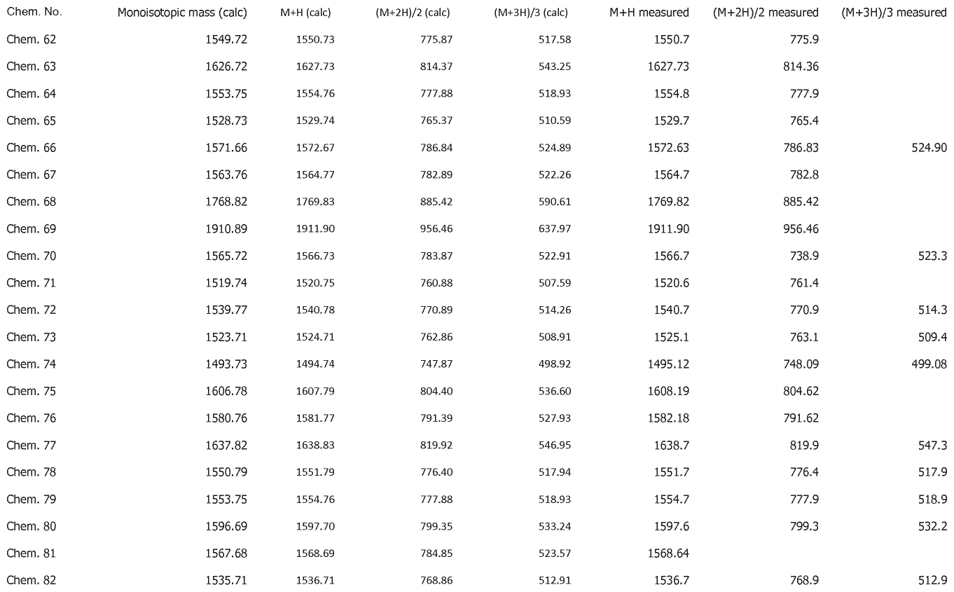

FIG. 1 shows mass spectrometry data for the compounds disclosed herein.

### Example 2: Melanocortin 4 Receptor (MC4R) Binding Assay

In Vitro ¹²⁵NDP-α-MSH Binding to Recombinant BHK Cells Expressing Human MC4 Receptor (Filtration Assay)

In order to assess the binding affinity of the selected compounds for the human MC4R competitive binding assays was performed with radiolabeled NDP-alpha MSH (¹²⁵NDP-α-MSH).

The assay was performed in 96-well filterplates (Unifilter) using BHK cells stably expressing the human MC4 receptor.

The BHK cell membranes were prepared from frozen or fresh cells that were homogenized in 20 mM HEPES pH 7.1, 5 mM MgCl₂, 1 mg/ml bacitracin (buffer 1) and centrifuged at 15000 rpm at 4° C., 10 min in a Sorvall RC 5B plus, SS-34 rotor. The supernatant was discarded, and the pellets were re-suspended in buffer 1, homogenized and centrifuged two more times. The final pellets were re-suspended in buffer 1 and the protein concentration was measured and adjusted with buffer 1 to 5 mg/ml and the membrane preparation was kept at −80° C. until assay. The assay was run on a dilution of this cell membrane suspension without any further preparation. The suspension was diluted to give maximally 10% specific binding, i.e. to approx. 50-100 fold dilution, optimized after each membrane preparation. The assay was performed in a total volume of 200 μl: 50 μl of cell suspension, 50 μl of ¹²⁵NDP-α-MSH (apx. 79 μM in final concentration), 50 μl of test compound and 50 μl binding buffer were mixed and incubated for 2 h at 25° C. (binding buffer: 25 mM HEPES, pH 7.0, 1 mM CaCl₂), 1 mM MgSO₄, 1 mM EGTA, 0.005% Tween™ 20 and 0.1% HSA or, alternatively, 0.1% ovalbumin (Sigma; catalogue No. A-5503)). Test compounds were dissolved in 100% DMSO and diluted in binding buffer in a dose range from 1 μM to 0.001 nM. Radiolabelled ligand and membranes were diluted in binding buffer. The incubation was stopped by dilution with 8×100 μl ice-cold 0.9% NaCl. The radioactivity retained on the filters was counted using a Packard Topcounter. The data were analysed by non-linear regression analysis of binding curves, using the Windows™ program GraphPad™ Prism (GraphPad Software, USA).

Results are presented in column 2 of table 1 in Example 4 below.

The experiment shows that the listed compounds generally show binding affinities towards the MC4R in the nanomolar range.

### Example 3: Functional Human Melanocortin Receptor 1 and 4 Assays

In order to assess the potency of the compounds on human MC1R and human MC4R cAMP signalling was measured.

cAMP induction was measured by c-AMPdynamic2 assay, HTRF 62AM4PEC system from CisBio for the human MC1R and human MC4R according to the protocol provided by the vendor. BHK cells expressing the human MC1 or MC4 receptor were generated by stable transfection with expression vectors encoding the cDNA of either MC1R or MC4R.

In details; 25 μl compound—or standard solution were added in the respective wells. Compound was reconstituted in buffer (DMEM w/o phenol red, 10 mM

Hepes, lx Glutamine, 0.1% OV-albumin1, 1 mM IBMX) and serially diluted starting at 2 μM, 10-fold seven times. Dilutions were carried out in the same aforementioned buffer to the desired working concentration ranges (2× higher than the final concentration). 25 μl cell solution was then added and allowed to incubate for 30 min while lightly shaking, at 25° C. If the cAMP response did not reach saturation level when starting at 1 uM, the compound was re-tested with dilutions starting at 20 μM. The provided components for the assay were reconstituted according to the CisBio assay protocol “3.1 supplied reagents and preparation procedure” in MilliQ purification system H₂O and diluted 1:20 in lysis buffer. 25 μl cAMP-d2 and 25 μl Cryptate conjugate were added to all wells.

Plates were centrifuged at 1500 rpm for 30 sec and incubated for 1 h, lightly shaking, at 25° C. in a hotel stacker. The plates were read on Mithras LB 940 provided by Berthold Technologies The data were analysed by non-linear regression analysis of binding curves, using the Windows™ program GraphPad™ Prism (GraphPad Software, USA).

Data is listed in the table as Ki or EC₅₀ values. EC₅₀ is a measure of the potency of a given compound and is defined herein as the half maximal effective concentration of the compound which induces a response (cAMP induction) halfway between the baseline and maximum response value after a fixed reaction time. Data listed as >, indicate that the highest concentration tested had no effect, where “no effect” is defined as the highest compound concentration with agonistic effect ≤5% of maximal response of the reference compound NDP-alpha-MSH. The latter is used when no full curve can be obtained in the concentration ranges indicated above. Results are presented in columns 3 and 4 of table 1 in Example 4 below. These experiments show that the majority of the listed compounds have potency values for activation of MC4R in the sub-nanomolar range and that the compounds have variable potency on MC1R.

### Example 4: Results from Examples 2 and 3

Table 1 below shows the results from Example 2 (column 2) and Example 3 (columns 3 and 4)

TABLE 1
Results

| | MC4R | | | MC1R/MC4R |
|---|---|---|---|---|
| | binding | MC4R cAMP | MC1R cAMP | cAMP EC₅₀ |
| Compound | Kᵢ (nM) | EC₅₀ (nM) | EC₅₀ (nM) | ratio |
| Chem. 1 | 47.9 | 14.94 | 198 | 13 |
| Chem. 2 | 0.07 | 0.01 | 1.54 | 154 |
| Chem. 3 | 0.42 | 0.26 | 7.29 | 28 |
| Chem. 4 | 0.69 | 0.09 | 0.67 | 7 |
| Chem. 5 | 0.99 | 0.21 | 6.47 | 31 |
| Chem. 6 | 0.19 | 0.06 | 2.35 | 39 |
| Chem. 7 | 0.47 | 0.06 | 4.56 | 76 |
| Chem. 8 | 0.08 | 0.02 | 1.76 | 88 |
| Chem. 9 | 0.06 | 0.02 | 4.02 | 201 |
| Chem. 10 | 0.13 | 0.05 | 4.88 | 98 |
| Chem. 11 | 0.25 | 0.03 | 2.17 | 72 |
| Chem. 12 | 0.28 | 0.07 | 3.97 | 57 |
| Chem. 13 | 0.26 | 0.1 | 6.36 | 64 |
| Chem. 14 | 30.19 | 3.55 | >10 | >3 |
| Chem. 15 | 0.12 | 0.09 | 5.43 | 60 |
| Chem. 16 | 0.34 | 0.21 | >1 | >5 |
| Chem. 17 | 0.83 | 0.36 | 103.84 | 288 |
| Chem. 18 | 0.38 | 0.12 | 4.22 | 35 |
| Chem. 19 | 1.49 | 0.13 | >1 | >8 |
| Chem. 20 | 0.15 | 0.04 | 4.97 | 124 |
| Chem. 21 | 3.79 | 0.43 | >4 | >9 |
| Chem. 22 | 0.06 | 0.02 | 0.22 | 11 |
| Chem. 23 | 13.62 | 3.26 | >10 | >3 |
| Chem. 24 | 0.55 | 0.49 | 178.35 | 364 |
| Chem. 25 | 0.04 | 0.02 | 0.34 | 17 |
| Chem. 26 | 0.44 | 0.18 | 17.2 | 96 |
| Chem. 27 | 1.01 | 0.24 | 50.94 | 212 |
| Chem. 28 | 892.5 | 518 | >100 | — |
| Chem. 29 | 1208 | 1017 | >100 | — |
| Chem. 30 | 0.09 | 0.03 | 0.61 | 5 |
| Chem. 31 | 1.67 | 0.29 | 7.78 | 27 |
| Chem. 32 | 0.37 | 0.12 | 1.98 | 17 |
| Chem. 33 | 27.75 | 19.82 | >1 | — |
| Chem. 34 | 0.88 | 0.12 | 2.6 | 22 |
| Chem. 35 | 0.03 | 0.01 | 0.28 | 28 |
| Chem. 36 | 0.12 | 0.04 | 1.05 | 26 |
| Chem. 37 | 3.31 | 1.57 | 13.06 | 8 |
| Chem. 38 | 5.64 | 4.22 | >3.7 | — |
| Chem. 39 | 1.8 | 1.54 | 56.36 | 37 |
| Chem. 40 | 0.31 | 0.29 | 11.1 | 38 |
| Chem. 41 | 5.39 | 1.34 | 137.03 | 102 |
| Chem. 42 | 10000 | >1000 | >100 | — |
| Chem. 43 | 2.61 | 0.44 | 46.9 | 107 |
| Chem. 44 | 50.85 | 1.53 | 31.93 | 21 |
| Chem. 45 | 1003 | 45.7 | >100 | >2 |
| Chem. 46 | 7.86 | 5.65 | >5.5 | >1 |
| Chem. 47 | 0.72 | 0.12 | 2.79 | 23 |
| Chem. 48 | 0.15 | 0.06 | 9.14 | 152 |
| Chem. 49 | 3.92 | 1.28 | >5.5 | >4 |
| Chem. 50 | 19.95 | 1.17 | 66.16 | 56 |
| Chem. 51 | 0.5 | 0.2 | 2.69 | 13 |
| Chem. 52 | 0.76 | 0.23 | 47.69 | 207 |
| Chem. 53 | 9.47 | 10.98 | >10 | >1 |
| Chem. 54 | 0.21 | 0.04 | 0.29 | 7 |
| Chem. 55 | 0.33 | 0.1 | 4.14 | 41 |
| Chem. 56 | 1.82 | 0.44 | 35.33 | 80 |
| Chem. 57 | 0.21 | 0.19 | 10.44 | 55 |
| Chem. 58 | 0.09 | 0.11 | 3.93 | 36 |
| Chem. 59 | 0.01 | 0.004 | 0.16 | 40 |
| Chem. 60 | 0.1 | 0.17 | 53.27 | 313 |
| Chem. 61 | 0.22 | 0.31 | >1 | >3 |
| Chem. 62 | 0.13 | 0.17 | >1 | >6 |
| Chem. 63 | 0.08 | 0.02 | 0.84 | 42 |
| Chem. 64 | 0.09 | 0.03 | 4.23 | 141 |
| Chem. 65 | 0.47 | 0.42 | 10.75 | 26 |
| Chem. 66 | 0.03 | 0.01 | 0.22 | 22 |
| Chem. 67 | 0.95 | 2.99 | >55 | >18 |
| Chem. 68 | 10.44 | 4.35 | 4.87 | 1 |
| Chem. 69 | 4.65 | 1.52 | 6.9 | 5 |
| Chem. 70 | 0.07 | 0.38 | 75.67 | 199 |
| Chem. 71 | 0.05 | 0.08 | 33.21 | 415 |
| Chem. 72 | 0.03 | 0.03 | 5.86 | 195 |
| Chem. 73 | 0.3 | 1.26 | >50 | >40 |
| Chem. 74 | 0.08 | 0.29 | >5.5 | 19 |
| Chem. 75 | 0.11 | 0.44 | 68.05 | 155 |
| Chem. 76 | 0.09 | 0.16 | 21.78 | 136 |
| Chem. 77 | 0.72 | 0.46 | 49.8 | 108 |
| Chem. 78 | 0.02 | 0.04 | 3.8 | 95 |
| Chem. 79 | 0.03 | 0.02 | 1.16 | 58 |
| Chem. 80 | 0.16 | 0.15 | 8.65 | 58 |
| Chem. 81 | 0.03 | 0.07 | 67.02 | 957 |
| Chem. 82 | 0.07 | 0.53 | 94.29 | 178 |
| Chem. 83 | 0.17 | 2.15 | >10 | >5 |
| Chem. 84 | 0.19 | 0.59 | 43.4 | 74 |
| Chem. 85 | 0.02 | 0.01 | 0.22 | 22 |
| Chem. 86 | 9.6 | 14.47 | 146.65 | 10 |
| Chem. 87 | 0.17 | 0.04 | 1.39 | 35 |
| Chem. 88 | 0.2 | 0.02 | 1.31 | 66 |
| Chem. 89 | 0.07 | 0.02 | 0.59 | 30 |
| Chem. 90 | 0.11 | 0.01 | 0.65 | 65 |
| Chem. 91 | 0.51 | 0.71 | >10 | >14 |
| Chem. 92 | 0.04 | 0.13 | 47.12 | 362 |
| Chem. 93 | 0.03 | 0.04 | 18.24 | 456 |
| Chem. 94 | 0.25 | 4.38 | 113.0 | 26 |
| Chem. 95 | 0.07 | 0.22 | 87.7 | 399 |
| Chem. 96 | 0.04 | 0.04 | 57.66 | 1442 |
| Chem. 97 | 0.05 | 0.19 | 23.14 | 122 |
| Chem. 98 | 0.6 | 2.7 | >55 | >20 |
| Chem. 99 | 0.1 | 0.39 | 5.29 | 14 |
| Chem. 100 | 0.04 | 0.02 | 18.49 | 925 |
| Chem. 101 | 0.05 | 0.04 | 0.19 | 5 |
| Chem. 102 | 10.1 | 0.49 | 14.32 | 29 |
| Chem. 103 | 0.06 | 0.05 | >70 | >1400 |
| Chem. 104 | 8.87 | 0.6 | >100 | 167 |
| Chem. 105 | 43.05 | 6.62 | >100 | >15 |
| Chem. 106 | 42.8 | 10.59 | >100 | >10 |
| Chem. 107 | 39.15 | 10.58 | >100 | >10 |
| Chem. 108 | 23.3 | 2.52 | >100 | >40 |
| Chem. 109 | 0.3 | 0.15 | >7 | >47 |
| Chem. 110 | 0.09 | 0.05 | 63.28 | 1266 |
| Chem. 111 | 0.04 | 0.01 | 0.31 | 31 |
| Chem. 112 | 1.21 | 0.19 | 30.20 | 159 |
| Chem. 113 | — | 0.02 | 42.23 | 2112 |
| Chem. 114 | — | 0.04 | 113.75 | 2844 |
| Chem. 115 | — | 0.10 | 134.00 | 1340 |
| Chem. 116 | — | 0.12 | 231.25 | 1927 |
| Chem. 117 | — | 0.20 | 168.25 | 841 |
| Chem. 118 | — | 3.53 | 4375.50 | 1240 |
| Chem. 119 | — | 0.06 | 99.58 | 1660 |
| Chem. 120 | — | 0.08 | 293.83 | 3673 |
| Chem. 121 | — | 0.24 | 188.75 | 786 |

Values (in nM) denoted by ‘>’ means highest dose as used in the assay with no response and is used when no meaningful EC₅₀ value can be calculated.

### Example 5: An In Vivo Food Intake Model

The MC4R system is central to appetite regulation and agonists are known inhibit food intake. In order to screen compounds in a relevant in vivo model, efficacy was assessed by determining the effect of the compound on reducing food intake acutely in mice.

Animals and Diet

All animal protocols were approved by an Institutional Animal Care and Use Committee and Ethical Review Committee of Novo Nordisk. Animals were housed according to Novo Nordisk rodent housing standards, and were given ad libitum access to food and water under controlled lighting (12 h:12 h light/dark cycle; lights off 11:00-23:00), temperature (23±2° C.) and relative humidity (50±20%) conditions. For each acute food intake study C57BL/6J male mice (25-30 grams body weight) were acclimated for two weeks to the appropriate light cycle and to single-housing (one mouse per cage) in cages linked to a BioDAQ automated food intake system (Research Diets Inc., New Brunswick, N.J. USA). The animals were also acclimated to new food (Research Diets 12450B, 10% kcal fat) during this time.

Dosing and Food Intake Measurements

The BioDAQ is a computer automated scientific instrument for Biological Data Acquisition of food intake in rodents and measures food intake from each cage every 15 minutes. A total of 32 animals can be accommodated in the system per study, therefore, animals were randomized into 4 groups of n=8 for each study. A 20 hour baseline food intake recording was taken prior to compound dosing in order to assess that all animals exhibited a normal food intake pattern. Animals were fasted ˜4 hr prior to dosing. Vehicle (10 mM phosphate; 140 mM sodium chloride) or compound was dosed at a volume of 10 ml per kg subcutaneously approximately 20 minutes prior to the start of the dark cycle (compound chem no. and dosages are indicated in table 2 below). After dosing, food intake was recorded for approximately 24 hours.

Results

The inhibitory effect of compounds on food intake was generally well-correlated with in vitro binding affinity on the human MC4 receptor and with plasma stability of the compound. Most studies were conducted as dose response studies with vehicle and the compound at 3 doses (0.3, 1 and 3 mg/kg). Some studies were done for the purpose of dose-range finding, while others were done to comparatively assess the effect of 3 compounds at the same dose. Maximal effects on food intake occurred within the first 1-5 hours post-dose. The comparative effect of compounds across studies on food intake was assessed by normalizing measurements as % effect on cumulative food intake after a single subcutaneous dose, relative to vehicle. Results are reported in Table 2 as % effect on food intake at 3 hours post dose.

TABLE 2
% effect on food intake at 3 hours post dose

| | | % Effect on food |
|---|---|---|
| | | intake at 3 hours |
| | | post dose, |
| | Dose | relative to |
| Chem no. | (mg/kg) | vehicle |
| 52 | 1 | −66 ± 9 |
| 70 | 1 | −39 ± 10 |
| 81 | 1 | −72 ± 7 |
| 96 | 1 | −17 ± 14 |
| 110 | 1 | −26 ± 15 |
| 111 | 1 | −63 ± 4 |
| 114 | 1 | −64 ± 6 |
| 120 | 1 | −28 ± 12 |

### Example 6: An In Vivo Obese Model

Stimulation of MC4R signaling is reported to modulate energy intake and expenditure in both humans and rodents. In order to test the effect of MC4R agonists on food intake and body weight, diet-induced obese (DIO) mice were treated once daily with compounds to assess weight loss efficacy.

Animals and Diet

All animal protocols were approved by an Institutional Animal Care and Use Committee and Ethical Review Committee of Novo Nordisk. Animals were housed according to Novo Nordisk rodent housing standards, and were given ad libitum access to food and water under controlled lighting (12 h:12 h light/dark cycle; lights off 17:00-05:00), temperature (23±2° C.) and relative humidity (50±20%) conditions. Diet-induced obese (DIO) male C57BL/6J mice maintained on a high fat diet (60% kcal fat, 5.24 kcal/gram; RD12492, Research Diets, New Brunswick, N.J., USA) for 22 weeks were obtained from Charles River (France). Upon arrival, the mice were single-housed (one mouse per cage) and allowed to acclimate to their new environment for two weeks prior to the start of treatment.

Group Allocation and Dosing

Prior to initiation of the study, animals were single-housed and acclimated to handling for 7 days. The DIO mice were distributed into groups (n=8/group) such that statistical variations in the mean and standard deviations of fat mass and body weight were minimized between groups. Animals were dosed once daily, subcutaneously at 16:00 with either vehicle or compound

Body Weight and Food Intake

Body weight and food intake were measured immediately prior to dosing each day. The average starting body weight of the mice prior to start of treatment was 47.2±0.2 grams.

Results: Effects of an MC4R Agonist on Weight Loss and Food Intake

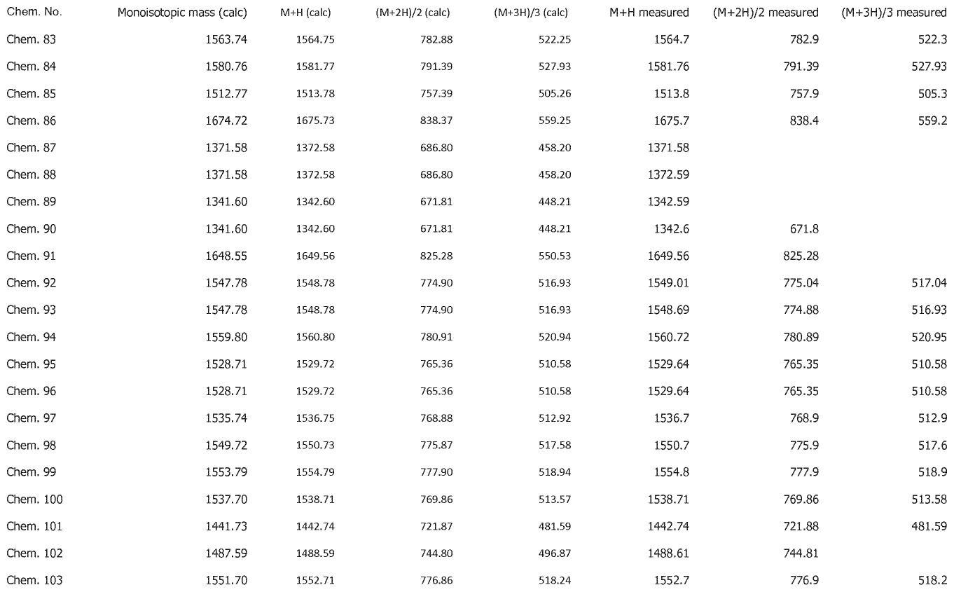

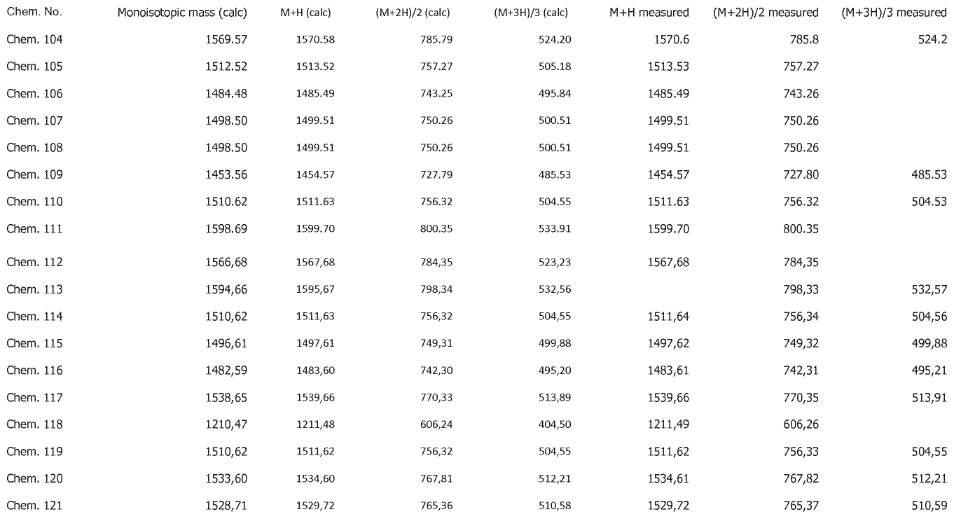

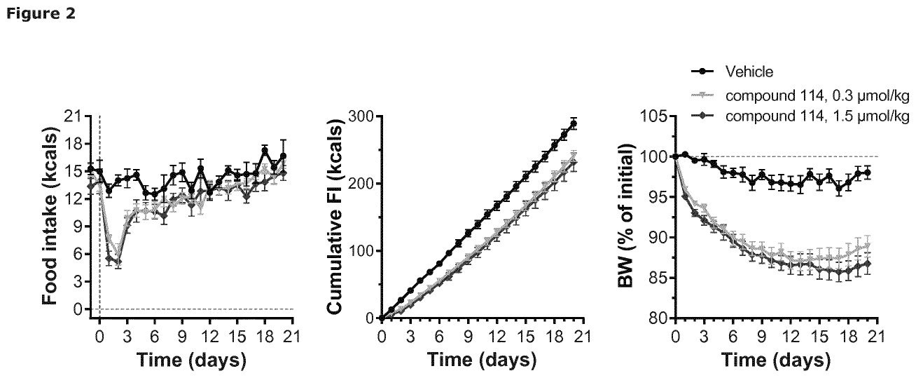

Results are shown in table 3 below and in FIG. 2. Once-daily subcutaneous treatment with the MC4R agonist, chem. 114 induced a 11.1±1.3% (0.3 μmol/kg) and 13.2±1.3% (1.5 μmol/kg) reduction in body weight, relative to initial body weight (100*BW_{day x}/BW_{day 0}). The reduction in body weight was concurrent with a transient reduction in food intake that returned to baseline levels during the second week of treatment. By the end of the study food intake was normalized to the level of vehicle controls and body weight loss was maintained relative to vehicle-treated animals.

TABLE 3
Daily and cumulative food intake, and relative body weight in DIO mice treated
once daily with vehicle or compound

| | Daily food intake, kcals | | | Cumulative food intake, kcals | | | Relative body weight, % initial | | |
|---|---|---|---|---|---|---|---|---|---|
| | | chem 114, | chem 114, | | chem 114, | chem 114, | | chem 114, | chem 114, |
| Time | | 0.3 | 1.5 | | 0.3 | 1.5 | | 0.3 | 1.5 |
| (days) | Vehicle | μmol/kg | μmol/kg | Vehicle | μmol/kg | μmol/kg | Vehicle | μmol/kg | μmol/kg |
| 0 | 15 ± 1.2 | 13.4 ± 0.6 | 13.9 ± 1.3 | — | — | — | — | — | — |
| 1 | 12.9 ± 0.7 | 7.7 ± 0.5 | 5.5 ± 0.8 | 12.9 ± 0.7 | 7.7 ± 0.5 | 5.5 ± 0.8 | 100.3 ± 0.3 | 96 ± 0.3 | 95.1 ± 0.4 |
| 2 | 14 ± 0.6 | 6 ± 1.2 | 5.2 ± 0.8 | 26.9 ± 1 | 13.6 ± 1.5 | 10.7 ± 1.4 | 99.5 ± 0.3 | 94.2 ± 0.3 | 93 ± 0.4 |
| 3 | 14.2 ± 1 | 9.9 ± 0.9 | 9.1 ± 0.8 | 41.1 ± 1.9 | 23.5 ± 2.1 | 19.8 ± 2.2 | 99.6 ± 0.7 | 93.6 ± 0.5 | 92.1 ± 0.6 |
| 4 | 14.6 ± 0.6 | 10.7 ± 0.7 | 10.7 ± 1.2 | 55.7 ± 2 | 34.3 ± 2.5 | 30.5 ± 3.1 | 99.1 ± 0.4 | 91.8 ± 0.7 | 91.3 ± 0.7 |
| 5 | 12.7 ± 0.8 | 10.5 ± 0.8 | 10.7 ± 0.9 | 68.3 ± 2.6 | 44.8 ± 2.9 | 41.1 ± 4 | 98.1 ± 0.8 | 91.1 ± 0.6 | 90.6 ± 0.9 |
| 6 | 12.5 ± 0.7 | 10.8 ± 0.9 | 10.6 ± 0.8 | 80.8 ± 3 | 55.6 ± 3.7 | 51.7 ± 4.6 | 98 ± 0.6 | 89.9 ± 0.9 | 89.6 ± 0.8 |
| 7 | 13.1 ± 1.5 | 12.1 ± 0.9 | 10.2 ± 1 | 96.6 ± 4.3 | 67.7 ± 4.3 | 61.9 ± 5.4 | 97.8 ± 0.9 | 89.5 ± 0.9 | 88.6 ± 0.9 |
| 8 | 14.6 ± 1.1 | 11.1 ± 0.6 | 11.9 ± 0.7 | 111.2 ± 5.1 | 78.8 ± 4.5 | 73.8 ± 5.8 | 96.7 ± 0.9 | 88.6 ± 0.9 | 87.8 ± 0.8 |
| 9 | 14.9 ± 1 | 12.1 ± 0.6 | 12.4 ± 0.8 | 126.1 ± 5.4 | 90.8 ± 4.7 | 86.2 ± 6.4 | 97.8 ± 0.6 | 88.6 ± 0.9 | 87.8 ± 1 |
| 10 | 12.9 ± 0.9 | 12.3 ± 0.8 | 11.3 ± 1.2 | 139 ± 6 | 103.2 ± 5.3 | 97.5 ± 7.5 | 96.8 ± 0.9 | 87.7 ± 1.1 | 87.1 ± 0.9 |
| 11 | 15.3 ± 1 | 11 ± 0.7 | 12.8 ± 1.4 | 154.3 ± 6.6 | 114.2 ± 5.8 | 110.4 ± 8.4 | 96.8 ± 1 | 88.2 ± 1.1 | 86.9 ± 0.9 |
| 12 | 12.7 ± 0.9 | 13.4 ± 0.6 | 12.9 ± 0.9 | 166.9 ± 6.8 | 127.6 ± 6.1 | 123.3 ± 9.2 | 96.6 ± 0.9 | 87.2 ± 1.1 | 86.6 ± 1.1 |
| 13 | 13.8 ± 0.6 | 13.2 ± 0.7 | 13.2 ± 0.7 | 180.8 ± 7.2 | 140.8 ± 6.4 | 136.4 ± 9.8 | 96.5 ± 1.1 | 87.1 ± 1.2 | 86.7 ± 1.3 |
| 14 | 15.1 ± 0.5 | 13.2 ± 0.7 | 12.9 ± 1.1 | 195.9 ± 7.2 | 153.9 ± 6.9 | 149.3 ± 10.7 | 97.8 ± 0.7 | 87.3 ± 1.3 | 86.7 ± 1.3 |
| 15 | 14.6 ± 0.6 | 13.6 ± 0.7 | 13.5 ± 0.6 | 210.4 ± 7.3 | 167.5 ± 7.5 | 162.8 ± 11.1 | 96.8 ± 1 | 87.4 ± 1.3 | 86 ± 1.2 |
| 16 | 14.7 ± 1.3 | 13.7 ± 0.5 | 12.2 ± 0.7 | 225.1 ± 8.1 | 181.2 ± 7.8 | 175.1 ± 11.7 | 97.6 ± 0.9 | 87.5 ± 1.3 | 86 ± 1.3 |
| 17 | 14.8 ± 1.1 | 14.5 ± 0.5 | 13.6 ± 0.8 | 239.9 ± 7.7 | 195.8 ± 7.9 | 188.7 ± 12.2 | 96 ± 1 | 87.4 ± 1.3 | 85.7 ± 1.2 |
| 18 | 17.3 ± 0.6 | 15.3 ± 0.6 | 13.8 ± 1 | 257.2 ± 7.9 | 211 ± 8.3 | 202.5 ± 13.1 | 96.8 ± 1 | 87.9 ± 1.4 | 85.9 ± 1.2 |
| 19 | 15.4 ± 0.8 | 14.3 ± 0.8 | 14.5 ± 0.9 | 272.6 ± 8.3 | 225.4 ± 8.7 | 217 ± 13.9 | 97.9 ± 0.9 | 88.6 ± 1.4 | 86.4 ± 1.3 |
| 20 | 16.7 ± 1.8 | 15.9 ± 0.5 | 14.8 ± 0.8 | 289.2 ± 8.8 | 241.2 ± 8.5 | 231.8 ± 13.6 | 98 ± 0.8 | 88.9 ± 1.3 | 86.8 ± 1.3 |

Data are mean ± SEM.

1. A bicyclic compound according to Formula I: 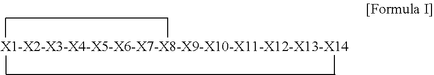
wherein
X1 and X8 are joined and X1 and X14 are joined;
X1 is Cys, HCys or Pen;
X2 is Ala, Pro, Hyp, THAZ, Aib, D-Ala, βAla, Sar, D-Pro, Val, D-Val, ACBC, GABA, ACP, Glu, Lys, D-Lys, Arg, AZE, PIP, OXA, Gly or absent;
X3 is His, Pro or Hyp;
X4 is D-Phe or Phe;
X5 is Arg, Lys, HArg, His, Dab, Dap, Cit, Orn, Arg(NO₂), N-MeArg, 4cis-GuaPro, 4trans-GuaPro, AGP, AcLys, Gln or Lys(Me)₂;
X6 is Trp, 2-Nal, 4-CN-Phe, 3,4-DiCl-Phe, 3,4-DiMeO-Phe or 1-Nal;
X7 is Ala, Glu, Gly, D-Ala, Arg or absent;
X8 is Cys, HCys or Pen;
X9 is Ala, Pro, Hyp, THAZ, Aib, D-Ala, βAla, Sar, D-Pro, Val, D-Val, ACBC, GABA, Arg, ACP, Glu, Lys, AZE, PIP, Orn, Gly, D-Lys or absent;
X10 is His, Pro, Hyp, Phe, Glu, Lys, D-Lys, Tyr, Ala, D-Ala, Asp, Arg or Orn;
X11 is D-Phe, Phe, Glu, Lys, Lys(CH₂COOH)₂, D-Lys, D-Ala, Ala, Arg, D-Arg or Asp;
X12 is Arg, Lys, HArg, His, Dab, Dap, Cit, Ala, Orn, Arg(NO₂), N-MeArg, 4cis-GuaPro, 4trans-GuaPro, AGP, Glu, Ala, Orn, D-Lys, Lys(Me)₂, Asn or absent;
X13 is Trp, Arg, 2-Nal, 4-CN-Phe, 3,4-DiCl-Phe, 3,4-DiMeO-Phe, 1-Nal, Ala, Phe or absent;
X14 is Ala, Glu, Gly, D-Ala, Arg, Phe or absent;
including all enantiomers and diastereomers thereof, or a pharmaceutically acceptable salt of any of the foregoing.

2. The bicyclic compound according to claim 1 wherein
X1 and X8 are joined by a disulphide bond or a methylene bridge, and
X1 and X14 are joined by an amide bond between the alpha amine of X1 and the alpha carboxylic group of X14.

3. The bicyclic compound according to claim 1 wherein said compound comprises 9-14 amino acid residues.

4. The bicyclic compound according to claim 1 wherein said compound is selected from the group consisting of chem. 1-121 (SEQ ID NOs:1-121).

5. The bicyclic compound according to claim 1 wherein
X1 and X8 are joined by a disulphide bond, and wherein
X1 is Cys, HCys or Pen;
X2 is THAZ or Pro;
X3 is His;
X4 is D-Phe;
X5 is His, Dab, or Dap;
X6 is Trp;
X7 is absent;
X8 is Cys, HCys or Pen;
X9 is Lys or Arg;
X10 is Ala;
X11 is D-Lys, D-Arg, or Arg;
X12 is Ala;
X13 is Trp; and
X14 is absent
including all enantiomers and diastereomers thereof, or a pharmaceutically acceptable salt of any of the foregoing.

6. The bicyclic compound according to claim 1 wherein said compound is selected from the group consisting of:
c[c[Cys-THAZ-His-D-Phe-His-Trp-Cys]-Lys-Ala-D-Lys-Ala-Trp] (SEQ ID NO:114);
and
c[c[Cys-THAZ-His-D-Phe-His-Trp-Cys]-Ala-His-Glu-Orn-Arg] (SEQ ID NO:120)
including all enantiomers and diastereomers thereof or a pharmaceutically acceptable salt of any of the foregoing.

7. (canceled)

8. (canceled)

9. (canceled)

10. (canceled)

11. A method of treating obesity or overweight, comprising administering to a patient in need thereof an effective amount of the bicyclic compound according to claim 1, optionally in combination with one or more additional therapeutically active compounds.

12. A method of regulating appetite, comprising administering to a patient in need thereof an effective amount of the bicyclic compound according to claim 1, optionally in combination with one or more additional therapeutically active compounds.

13. A method of preventing or reducing weight gain after successfully having lost weight, comprising administering to a patient in need thereof an effective amount of the bicyclic compound according to claim 1, optionally in combination with one or more additional therapeutically active compounds.

14. A method of treating a disease or state related to overweight or obesity, comprising administering to a patient in need thereof an effective amount of the bicyclic compound according to claim 1, optionally in combination with one or more additional therapeutically active compounds.

15. A pharmaceutical composition comprising a bicyclic compound according to claim 1 and one or more excipients.

16. A pharmaceutical composition comprising a bicyclic compound according to claim 6 and one or more excipients.

17. The bicyclic compound according to claim 2, wherein said compound comprises 9-14 amino acid residues.


SEQUENCE LISTING (121 sequences)

SEQ ID NO: 1 - protein, 12 aa, Artificial sequence
  Synthetic
  MISC_FEATURE (1)..(12): /note=“Cyclic peptide”
  DISULFID (1)..(7)
  MOD_RES (3)..(3): D-amino acid
  MOD_RES (9)..(9): D-amino acid
CHFRWECHFRWE

SEQ ID NO: 2 - protein, 12 aa, Artificial sequence
  Synthetic
  MISC_FEATURE (1)..(12): /note=“Cyclic peptide”
  DISULFID (1)..(7)
  MOD_RES (4)..(4): D-amino acid
  MOD_RES (10)..(10): D-amino acid
CGHFRWCGHFRW

SEQ ID NO: 3 - protein, 12 aa, Artificial sequence
  Synthetic
  MISC_FEATURE (1)..(12): /note=“Cyclic peptide”
  DISULFID (1)..(7)
  MOD_RES (1)..(1): Homocysteine
  MOD_RES (4)..(4): D-amino acid
  MOD_RES (7)..(7): Homocysteine
  MOD_RES (10)..(10): D-amino acid
XAHFRWXAHFRW

SEQ ID NO: 4 - protein, 12 aa, Artificial sequence
  Synthetic
  MISC_FEATURE (1)..(12): /note=“Cyclic peptide”
  DISULFID (1)..(7)
  MOD_RES (3)..(3): D-amino acid
  MOD_RES (9)..(9): D-amino acid
CHFRWGCHFRWG

SEQ ID NO: 5 - protein, 12 aa, Artificial sequence
  Synthetic
  MISC_FEATURE (1)..(12): /note=“Cyclic peptide”
  DISULFID (1)..(7)
  MOD_RES (3)..(3): D-amino acid
  MOD_RES (9)..(9): D-amino acid
CHFRWACHFRWA

SEQ ID NO: 6 - protein, 12 aa, Artificial sequence
  Synthetic
  DISULFID (1)..(7)
  MISC_FEATURE (1)..(12): /note=“Cyclic peptide”
  MOD_RES (3)..(3): D-amino acid
  MOD_RES (6)..(6): D-amino acid
  MOD_RES (9)..(9): D-amino acid
  MOD_RES (12)..(12): D-amino acid
CHFRWACHFRWA

SEQ ID NO: 7 - protein, 12 aa, Artificial sequence
  Synthetic
  MISC_FEATURE (1)..(12): /note=“Cyclic peptide”
  MOD_RES (4)..(4): D-amino acid
  MOD_RES (10)..(10): D-amino acid
AAHFRWAAHFRW

SEQ ID NO: 8 - protein, 12 aa, Artificial sequence
  Synthetic
  MISC_FEATURE (1)..(7): /note=“Methylene bridge”
  MISC_FEATURE (1)..(12): /note=“Cyclic peptide”
  MOD_RES (4)..(4): D-amino acid
  MOD_RES (10)..(10): D-amino acid
CAHFRWCAHFRW

SEQ ID NO: 9 - protein, 12 aa, Artificial sequence
  Synthetic
  MISC_FEATURE (1)..(12): /note=“Cyclic peptide”
  DISULFID (1)..(7)
  MOD_RES (2)..(2): 2-amino isobutyric acid
  MOD_RES (4)..(4): D-amino acid
  MOD_RES (8)..(8): 2-amino isobutyric acid
  MOD_RES (10)..(10): D-amino acid
CXHFRWCXHFRW

SEQ ID NO: 10 - protein, 12 aa, Artificial sequence
  Synthetic
  MISC_FEATURE (1)..(12): /note=“Cyclic peptide”
  DISULFID (1)..(7)
  MOD_RES (2)..(2): D-amino acid
  MOD_RES (4)..(4): D-amino acid
  MOD_RES (8)..(8): D-amino acid
  MOD_RES (10)..(10): D-amino acid
CAHFRWCAHFRW

SEQ ID NO: 11 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CAHFRWCAAFAW

SEQ ID NO: 12 - protein, 12 aa, Artificial sequence
  Synthetic
  MISC_FEATURE (1)..(12): /note=“Cyclic peptide”
  DISULFID (1)..(7)
  MOD_RES (4)..(4): D-amino acid
CAHFRWCRFFNA

SEQ ID NO: 13 - protein, 13 aa, Artificial sequence
  Synthetic
  MISC_FEATURE (1)..(13): /note=“Cyclic peptide”
  DISULFID (1)..(7)
  MOD_RES (4)..(4): D-amino acid
CAHFRWCRFFNAF

SEQ ID NO: 14 - protein, 12 aa, Artificial sequence
  Synthetic
  DISULFID (1)..(7)
  MISC_FEATURE (1)..(12): /note=“Cyclic peptide”
  MOD_RES (3)..(3): Hydroxyproline
  MOD_RES (4)..(4): D-amino acid
  MOD_RES (10)..(10): D-amino acid
CAXFRWCAXFRW

SEQ ID NO: 15 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CAHFKWCAHFKW

SEQ ID NO: 16 - protein, 12 aa, Artificial sequence
  Synthetic
  DISULFID (1)..(7)
  MISC_FEATURE (1)..(12)
  MOD_RES (4)..(4): D-amino acid
  MOD_RES (5)..(5): Ornithine
  MOD_RES (10)..(10): D-amino acid
  MOD_RES (11)..(11): Ornithine
CAHFXWCAHFXW

SEQ ID NO: 17 - protein, 12 aa, Artificial sequence
  Synthetic
  DISULFID (1)..(7)
  MISC_FEATURE (1)..(12)
  MOD_RES (4)..(4): D-amino acid
  MOD_RES (5)..(5): Diamino butyric acid
  MOD_RES (10)..(10): D-amino acid
  MOD_RES (11)..(11): Diamino butyric acid
CAHFXWCAHFXW

SEQ ID NO: 18 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): N,N-dimethyl-lysine
  MOD_RES (10)..(10): D-amino acid
  MOD_RES (11)..(11): N,N-dimethyl-lysine
CAHFXWCAHFXW

SEQ ID NO: 19 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): Citrulline
  MOD_RES (10)..(10): D-amino acid
  MOD_RES (11)..(11): Citrulline
CAHFXWCAHFXW

SEQ ID NO: 20 - protein, 12 aa, Artificial sequence
  Synthetic
  DISULFID (1)..(7)
  MISC_FEATURE (1)..(12): /note=“Cyclic peptide”
  MOD_RES (2)..(2): 1-amino cyclobutane carboxylic acid
  MOD_RES (4)..(4): D-amino acid
  MOD_RES (8)..(8): 1-amino cyclobutane carboxylic acid
  MOD_RES (10)..(10): D-amino acid
CXHFRWCXHFRW

SEQ ID NO: 21 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): Omega nitro arginine
  MOD_RES (10)..(10): D-amino acid
  MOD_RES (11)..(11): Omega nitro arginine
CAHFXWCAHFXW

SEQ ID NO: 22 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): Homo-arginine
  MOD_RES (10)..(10): D-amino acid
  MOD_RES (11)..(11): Homo-arginine
CAHFXWCAHFXW

SEQ ID NO: 23 - protein, 12 aa, Artificial sequence
  Synthetic
  MISC_FEATURE (1)..(7): /note=“Methylene bridge”
  MISC_FEATURE (1)..(12): /note=“Cyclic peptide”
  MOD_RES (3)..(3): Hydroxyproline
  MOD_RES (4)..(4): D-amino acid
  MOD_RES (9)..(9): Hydroxyproline
  MOD_RES (10)..(10): D-amino acid
CAXFRWCAXFRW

SEQ ID NO: 24 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CAHFHWCAHFHW

SEQ ID NO: 25 - protein, 12 aa, Artificial sequence
  Synthetic
  DISULFID (1)..(7)
  MISC_FEATURE (1)..(12): /note=“Cyclic peptide”
  MOD_RES (2)..(2): Beta alanine
  MOD_RES (4)..(4): D-amino acid
  MOD_RES (8)..(8): Beta alanine
  MOD_RES (10)..(10): D-amino acid
CXHFRWCXHFRW

SEQ ID NO: 26 - protein, 10 aa, Artificial sequence
  Synthetic
  MISC_FEATURE (1)..(10): /note=“Cyclic peptide”
  MISC_FEATURE (1)..(6): /note=“Methylene bridge”
  MOD_RES (2)..(2): Hydroxyproline
  MOD_RES (3)..(3): D-amino acid
  MOD_RES (7)..(7): Hydroxyproline
  MOD_RES (8)..(8): D-amino acid
CXFRWCXFRW

SEQ ID NO: 27 - protein, 10 aa, Artificial sequence
  Synthetic
  DISULFID (1)..(6)
  MISC_FEATURE (1)..(10): /note=“Cyclic peptide”
  MOD_RES (2)..(2): Hydroxyproline
  MOD_RES (3)..(3): D-amino acid
  MOD_RES (7)..(7): Hydroxyproline
  MOD_RES (8)..(8): D-amino acid
CXFRWCXFRW

SEQ ID NO: 28 - protein, 12 aa, Artificial sequence
  Synthetic
  DISULFID (1)..(7)
  DISULFID (1)..(12): /note=“Cyclic peptide”
  MOD_RES (4)..(4): D-amino acid
  MOD_RES (5)..(5): (2S,4R)-4-guanidinopyrrolidine-2-carboxylic acid
  MOD_RES (10)..(10): D-amino acid
  MOD_RES (11)..(11): (2S,4R)-4-guanidinopyrrolidine-2-carboxylic acid
CAHFXWCAHFXW

SEQ ID NO: 29 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): (2S,4S)-4-guanidinopyrrolidine-2-carboxylic acid
  MOD_RES (10)..(10): D-amino acid
  MOD_RES (11)..(11): (2S,4S)-4-guanidinopyrrolidine-2-carboxylic acid
CAHFXWCAHFXW

SEQ ID NO: 30 - protein, 12 aa, Artificial sequence
  Stnthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CPHFRWCPHFRW

SEQ ID NO: 31 - protein, 12 aa, Artificial sequence
  Synthetic
  DISULFID (1)..(7)
  MISC_FEATURE (1)..(12): /note=“Cyclic peptide”
  MOD_RES (2)..(2): Sarcosine
  MOD_RES (4)..(4): D-amino acid
  MOD_RES (8)..(8): Sarcosine
  MOD_RES (10)..(10): D-amino acid
CXHFRWCXHFRW

SEQ ID NO: 32 - protein, 12 aa, Artificial sequence
  Synthetic
  DISULFID (1)..(7)
  MISC_FEATURE (1)..(12): /note=“Cyclic peptide”
  MOD_RES (2)..(2): Hydroxyproline
  MOD_RES (4)..(4): D-amino acid
  MOD_RES (8)..(8): Hydroxyproline
  MOD_RES (10)..(10): D-amino acid
CXHFRWCXHFRW

SEQ ID NO: 33 - protein, 12 aa, Artificial sequence
  Synthetic
  DISULFID (1)..(7)
  MISC_FEATURE (1)..(12): /note=“Cyclic peptide”
  MOD_RES (2)..(2): D-amino acid
  MOD_RES (4)..(4): D-amino acid
  MOD_RES (8)..(8): D-amino acid
  MOD_RES (10)..(10): D-amino acid
CPHFRWCPHFRW

SEQ ID NO: 34 - protein, 12 aa, Artificial sequence
  Synthetic
  DISULFID (1)..(7)
  MISC_FEATURE (1)..(12): /note=“Cyclic peptide”
  MOD_RES (2)..(2): D-amino acid
  MOD_RES (4)..(4): D-amino acid
  MOD_RES (8)..(8): D-amino acid
  MOD_RES (10)..(10): D-amino acid
CVHFRWCVHFRW

SEQ ID NO: 35 - protein, 12 aa, Artificial sequence
  Synthetic
  DISULFID (1)..(7)
  MISC_FEATURE (1)..(12): /note=“Cyclic peptide”
  MOD_RES (2)..(2): Gamma amino butyric acid
  MOD_RES (4)..(4): D-amino acid
  MOD_RES (8)..(8): Gamma amino butyric acid
  MOD_RES (10)..(10): D-amino acid
CXHFRWCXHFRW

SEQ ID NO: 36 - protein, 12 aa, Artificial sequence
  Synthetic
  MISC_FEATURE (1)..(12): /note=“Cyclic peptide”
  DISULFID (1)..(7)
  MOD_RES (4)..(4): D-amino acid
  MOD_RES (10)..(10): D-amino acid
CVHFRWCVHFRW

SEQ ID NO: 37 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): N-methyl arginine
  MOD_RES (10)..(10): D-amino acid
  MOD_RES (11)..(11): N-methyl arginine
CAHFXWCAHFXW

SEQ ID NO: 38 - protein, 12 aa, Artificial sequence
  Synthetic
  DISULFID (1)..(7)
  MISC_FEATURE (1)..(12): /note=“Cyclic peptide”
  MOD_RES (2)..(2): 2-amino isobutyric acid
  MOD_RES (3)..(3): Hydroxyproline
  MOD_RES (4)..(4): D-amino acid
  MOD_RES (8)..(8): 2-amino isobutyric acid
  MOD_RES (9)..(9): Hydroxyproline
  MOD_RES (10)..(10): D-amino acid
CXXFRWCXXFRW

SEQ ID NO: 39 - protein, 12 aa, Artificial sequence
  Synthetic
  DISULFID (1)..(7)
  MISC_FEATURE (1)..(12): /note=“Cyclic peptide”
  MOD_RES (2)..(2): 2-amino isobutyric acid
  MOD_RES (4)..(4): D-amino acid
  MOD_RES (8)..(8): 2-amino isobutyric acid
  MOD_RES (10)..(10): D-amino acid
CXPFRWCXPFRW

SEQ ID NO: 40 - protein, 12 aa, Artificial sequence
  Synthetic
  DISULFID (1)..(7)
  MISC_FEATURE (1)..(12): /note=“Cyclic peptide”
  MOD_RES (2)..(2): 2-amino isobutyric acid
  MOD_RES (4)..(4): D-amino acid
  MOD_RES (8)..(8): 2-amino isobutyric acid
  MOD_RES (10)..(10): D-amino acid
CXHFKWCXHFKW

SEQ ID NO: 41 - protein, 12 aa, Artificial sequence
  Synthetic
  DISULFID (1)..(7)
  MISC_FEATURE (1)..(12): /note=“Cyclic peptide”
  MOD_RES (2)..(2): 2-amino isobutyric acid
  MOD_RES (4)..(4): D-amino acid
  MOD_RES (5)..(5): Citrulline
  MOD_RES (8)..(8): 2-amino isobutyric acid
  MOD_RES (10)..(10): D-amino acid
  MOD_RES (11)..(11): Citrulline
CXHFXWCXHFXW

SEQ ID NO: 42 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): 2-amino-3-guanidino-propionic acid
  MOD_RES (10)..(10): D-amino acid
  MOD_RES (11)..(11): 2-amino-3-guanidino-propionic acid
CAHFXWCAHFXW

SEQ ID NO: 43 - protein, 12 aa, Artificial sequence
  Synthetic
  DISULFID (1)..(7)
  MISC_FEATURE (1)..(12): /note=“Cyclic peptide”
  MOD_RES (2)..(2): 2-amino isobutyric acid
  MOD_RES (4)..(4): D-amino acid
  MOD_RES (5)..(5): 2-amino-3-guanidino-propionic acid
  MOD_RES (8)..(8): 2-amino isobutyric acid
  MOD_RES (10)..(10): D-amino acid
  MOD_RES (11)..(11): 2-amino-3-guanidino-propionic acid
CXHFXWCXHFXW

SEQ ID NO: 44 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CAHFRWCAEEEW

SEQ ID NO: 45 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): Citrulline
CAHFXWCAEEEW

SEQ ID NO: 46 - protein, 12 aa, Artificial sequence
  Synthetic
  DISULFID (1)..(7)
  MISC_FEATURE (1)..(12): /note=“Cyclic peptide”
  MOD_RES (2)..(2): 2-amino isobutyric acid
  MOD_RES (4)..(4): D-amino acid
  MOD_RES (5)..(5): Diamino butyric acid
  MOD_RES (8)..(8): 2-amino isobutyric acid
  MOD_RES (10)..(10): D-amino acid
  MOD_RES (11)..(11): Diamino butyric acid
CXHFXWCXHFXW

SEQ ID NO: 47 - protein, 12 aa, Artificial sequence
  Synthetic
  DISULFID (1)..(7)
  MISC_FEATURE (1)..(12): /note=“Cyclic peptide”
  MOD_RES (2)..(2): 4-aminopiperidine-4-carboxylic acid
  MOD_RES (4)..(4): D-amino acid
  MOD_RES (8)..(8): 4-aminopiperidine-4-carboxylic acid
  MOD_RES (10)..(10): D-amino acid
CXHFRWCXHFRW

SEQ ID NO: 48 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): Citrulline
CAHFXWCAKKKW

SEQ ID NO: 49 - protein, 10 aa, Artificial sequence
  Synthetic
  DISULFID (1)..(6)
  MISC_FEATURE (1)..(12): /note=“Cyclic peptide”
  MOD_RES (3)..(3): D-amino acid
  MOD_RES (8)..(8): D-amino acid
CHFRWCHFRW

SEQ ID NO: 50 - protein, 12 aa, Artificial sequence
  Synthetic
  DISULFID (1)..(7)
  MISC_FEATURE (1)..(12): /note=“Cyclic peptide”
  MOD_RES (2)..(2): 4-aminopiperidine-4-carboxylic acid
  MOD_RES (4)..(4): D-amino acid
  MOD_RES (8)..(8): 4-aminopiperidine-4-carboxylic acid
  MOD_RES (10)..(10): D-amino acid
CXHFHWCXHFHW

SEQ ID NO: 51 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CEHFRWCEHFRW

SEQ ID NO: 52 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CKHFHWCKHFHW

SEQ ID NO: 53 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CEHFHWCEHFHW

SEQ ID NO: 54 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): Citrulline
  MOD_RES (10)..(10): D-amino acid
  MOD_RES (11)..(11): Citrulline
CRHFXWCRHFXW

SEQ ID NO: 55 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): Citrulline
  MOD_RES (10)..(10): D-amino acid
  MOD_RES (11)..(11): Citrulline
CKHFXWCKHFXW

SEQ ID NO: 56 - protein, 12 aa, Artificial sequence
  Synthetic
  DISULFID (1)..(7)
  MISC_FEATURE (1)..(12): /note=“Cyclic peptide”
CAHFRWCAHFRW

SEQ ID NO: 57 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CAHFQWCAKKKW

SEQ ID NO: 58 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): N-acetyl-lysine
  MOD_RES (10)..(10): D-amino acid
CAHFXWCAKKKW

SEQ ID NO: 59 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CAHFRWCAKKKR

SEQ ID NO: 60 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CAHFHWCAKKKW

SEQ ID NO: 61 - protein, 12 aa, Artificial sequence
  Synthetic
  DISULFID (1)..(7)
  MISC_FEATURE (1)..(12): /note=“Cyclic peptide”
  MOD_RES (2)..(2): 2-amino isobutyric acid
  MOD_RES (4)..(4): D-amino acid
CXHFHWCAKKKW

SEQ ID NO: 62 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CPHFHWCAKKKW

SEQ ID NO: 63 - protein, 12 aa, Artificial sequence
  Synthetic
  MOD_RES (1)..(1): Penicillamine
  DISULFID (1)..(7)
  MISC_FEATURE (1)..(12): /note=“Cyclic peptide”
  MOD_RES (4)..(4): D-amino acid
  MOD_RES (10)..(10): D-amino acid
XAHFRWCAHXRW

SEQ ID NO: 64 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (6)..(6): 1-naphthylalanine
CAHFRXCAKKKW

SEQ ID NO: 65 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (6)..(6): 4-cyano-phenylalanine
CAHFRXCAKKKW

SEQ ID NO: 66 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (6)..(6): 3,4-Dichloro-phenylalanine
CAHFRXCAKKKW

SEQ ID NO: 67 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (6)..(6): 3,4-dimethoxy-phenylalanine
CAHFRXCAKKKW

SEQ ID NO: 68 - protein, 12 aa, Artificial sequence
  Synthetic
  DISULFID (1)..(7)
  MISC_FEATURE (1)..(12): /note=“Cyclic peptide”
  MOD_RES (3)..(3): D-amino acid
  MOD_RES (9)..(9): D-amino acid
CHFRWRCHFRWR

SEQ ID NO: 69 - protein, 14 aa, Artificial sequence
  Synthetic
  DISULFID (1)..(8)
  MISC_FEATURE (1)..(14): /note=“Cyclic peptide”
  MOD_RES (4)..(4): D-amino acid
  MOD_RES (11)..(11): D-amino acid
CAHFRWRCAHFRWR

SEQ ID NO: 70 - protein, 12 aa, Artificial sequence
  Synthetic
  DISULFID (1)..(7)
  MISC_FEATURE (1)..(12): /note=“Cyclic peptide”
  MOD_RES (2)..(2): Hydroxyproline
  MOD_RES (4)..(4): D-amino acid
CXHFHWCAKKKW

SEQ ID NO: 71 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CPHFHWCAKKKR

SEQ ID NO: 72 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): Citrulline
CPHFXWCAKKKR

SEQ ID NO: 73 - protein, 12 aa, Artificial sequence
  Synthetic
  DISULFID (1)..(7)
  MISC_FEATURE (1)..(12): /note=“Cyclic peptide”
  MOD_RES (2)..(2): Sarcosine
  MOD_RES (4)..(4): D-amino acid
CXHFHWCAKKKW

SEQ ID NO: 74 - protein, 12 aa, Artificial sequence
  Synthetic
  DISULFID (1)..(7)
  MISC_FEATURE (1)..(12): /note=“Cyclic peptide”
  MOD_RES (2)..(2): Sarcosine
  MOD_RES (4)..(4): D-amino acid
CXHFHWCAKKKR

SEQ ID NO: 75 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CPHFHWCKKKKW

SEQ ID NO: 76 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CKHFHWCAKKKW

SEQ ID NO: 77 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CKHFHWCKKKKW

SEQ ID NO: 78 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CKHFHWCAKKKR

SEQ ID NO: 79 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (6)..(6): 2-naphthylalanine
CAHFRXCAKKKW

SEQ ID NO: 80 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CAHFRWCAYFKW

SEQ ID NO: 81 - protein, 12 aa, Artificial sequence
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
CXHFHWCAKKKW

SEQ ID NO: 82 - protein, 12 aa, Artificial sequence
  Synthetic
  DISULFID (1)..(7)
  MISC_FEATURE (1)..(12): /note=“Cyclic peptide”
  MOD_RES (2)..(2): Azetidine-2-carboxylic acid
  MOD_RES (4)..(4): D-amino acid
CXHFHWCAKKKW

SEQ ID NO: 83 - protein, 12 aa, Artificial sequence
  Synthetic
  DISULFID (1)..(7)
  MISC_FEATURE (1)..(12): /note=“Cyclic peptide”
  MOD_RES (2)..(2): Piperidine-2-carboxylic acid
  MOD_RES (4)..(4): D-amino acid
CXHFHWCAKKKW

SEQ ID NO: 84 - protein, 12 aa, Artificial sequence
  Synthetic
  DISULFID (1)..(7)
  MISC_FEATURE (1)..(12): /note=“Cyclic peptide”
  MOD_RES (2)..(2): D-amino acid
  MOD_RES (4)..(4): D-amino acid
CKHFHWCAKKKW

SEQ ID NO: 85 - protein, 12 aa, Artificial sequence
  Synthetic
  DISULFID (1)..(7)
  MISC_FEATURE (1)..(12): /note=“Cyclic peptide”
  MOD_RES (2)..(2): D-amino acid
  MOD_RES (4)..(4): D-amino acid
CAHFRWCAKKKR

SEQ ID NO: 86 - protein, 12 aa, Artificial sequence
  Synthetic
  DISULFID (1)..(7)
  MISC_FEATURE (1)..(12): /note=“Cyclic peptide”
  MOD_RES (2)..(2): D-amino acid
  MOD_RES (4)..(4): D-amino acid
  MOD_RES (8)..(8): D-amino acid
  MOD_RES (10)..(10): D-amino acid
CKHFHWCKHFHW

SEQ ID NO: 87 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CAHFRWCAAAAW

SEQ ID NO: 88 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CAHFRWCAAAAW

SEQ ID NO: 89 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CAHFRWCAAAAR

SEQ ID NO: 90 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CAHFRWCAAAAR

SEQ ID NO: 91 - protein, 12 aa, Artificial sequence
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
  MOD_RES (8)..(8): Thiazolidine-4-carboxylic acid
  MOD_RES (10)..(10): D-amino acid
CXHFHWCXHFHW

SEQ ID NO: 92 - protein, 12 aa, Artificial sequence
  Synthetic
  MOD_RES (1)..(1): Penicillamine
  DISULFID (1)..(7)
  MISC_FEATURE (1)..(12): /note=“Cyclic peptide”
  MOD_RES (4)..(4): D-amino acid
XPHFHWCAKKKR

SEQ ID NO: 93 - protein, 12 aa, Artificial sequence
  Synthetic
  MISC_FEATURE (1)..(12): /note=“Cyclic peptide”
  DISULFID (1)..(7)
  MOD_RES (4)..(4)
  MOD_RES (7)..(7): Penicillamine
CPHFHWXAKKKR

SEQ ID NO: 94 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (6)..(6): 3,4-dimethoxy-phenylalanine
CPHFRXCAKKKR

SEQ ID NO: 95 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CPHFHWCAKKHR

SEQ ID NO: 96 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CPHFHWCAHKKR

SEQ ID NO: 97 - protein, 12 aa, Artificial sequence
  Synthetic
  DISULFID (1)..(7)
  MISC_FEATURE (1)..(12): /note=“Cyclic peptide”
  MOD_RES (2)..(2): Hydroxyproline
  MOD_RES (4)..(4): D-amino acid
CXHFHWCAKKKR

SEQ ID NO: 98 - protein, 12 aa, Artificial sequence
  Synthetic
  DISULFID (1)..(7)
  MISC_FEATURE (1)..(12)
  MOD_RES (2)..(2): D-amino acid
  MOD_RES (4)..(4): D-amino acid
CPHFHWCAKKKW

SEQ ID NO: 99 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (5)..(5): Citrulline
CPHFXWCAKKKR

SEQ ID NO: 100 - protein, 12 aa, Artificial sequence
  Synthetic
  MISC_FEATURE (1)..(12): /note=“Cyclic peptide”
  DISULFID (1)..(7)
  MOD_RES (2)..(2): Thiazolidine-4-carboxylic acid
  MOD_RES (4)..(4): D-amino acid
CXHFHWCAKKKR

SEQ ID NO: 101 - protein, 11 aa, Artificial sequence
  Synthetic
  DISULFID (1)..(7)
  MISC_FEATURE (1)..(12): /note=“Cyclic peptide”
  MISC_FEATURE (1)..(11): /note=“Cyclic peptide”
  MOD_RES (4)..(4): D-amino acid
CAHFRWCKKKR

SEQ ID NO: 102 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
CAHFRWCAEEAW

SEQ ID NO: 103 - protein, 12 aa, Artificial sequence
  Synthetic
  DISULFID (1)..(7)
  MISC_FEATURE (1)..(12): /note=“Cyclic peptide”
  MOD_RES (2)..(2): Oxazolidine-4-carboxylic acid
  MOD_RES (4)..(4): D-amino acid
CXHFHWCAKKKW

SEQ ID NO: 104 - protein, 12 aa, Artificial sequence
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
  MOD_RES (10)..(10): N,N-bis(carboxymehtyl)-lysine
  MOD_RES (10)..(10): N,N-bis(carboxymethyl)-lysine
CXHFHWCAAXAW

SEQ ID NO: 105 - protein, 12 aa, Artificial sequence
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
CXHFHWCAEEAW

SEQ ID NO: 106 - protein, 12 aa, Artificial sequence
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
CXHFHWCADDAW

SEQ ID NO: 107 - protein, 12 aa, Artificial sequence
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
CXHFHWCADEAW

SEQ ID NO: 108 - protein, 12 aa, Artificial sequence
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
CXHFHWCAEDAW

SEQ ID NO: 109 - protein, 12 aa, Artificial sequence
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
CXHFHWCAAKAW

SEQ ID NO: 110 - protein, 12 aa, Artificial sequence
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
CXHFHWCAKKAW

SEQ ID NO: 111 - protein, 12 aa, Artificial sequence
  Synthetic
  DISULFID (1)..(7)
  MISC_FEATURE (1)..(12): /note=“Cyclic peptide”
  MOD_RES (4)..(4): D-amino acid
  MOD_RES (10)..(10): D-amino acid
CAHFRWCAHFRW

SEQ ID NO: 112 - protein, 12 aa, Artificial
  Synthetic
  MISC_FEATURE (1)..(12): /note=“Cyclic peptide”
  DISULFID (1)..(7)
  MOD_RES (4)..(4): D-amino acid
CAHFRWCPPERW

SEQ ID NO: 113 - protein, 12 aa, Artificial
  Synthetic
  DISULFID (1)..(7)
  MISC_FEATURE (1)..(12): /note=“Cyclc peptide”
  MOD_RES (2)..(2): Thiazolidine-4-carboxylic acid
  MOD_RES (4)..(4): D-amino acid
CXHFHWCARERR

SEQ ID NO: 114 - protein, 12 aa, Artificial
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
  MOD_RES (10)..(10): D-amino acid
CXHFHWCKAKAW

SEQ ID NO: 115 - protein, 12 aa, Artificial
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
  MOD_RES (9)..(9): Ornithine
CXHFHWCKXAAW

SEQ ID NO: 116 - protein, 12 aa, Artificial
  Synthetic
  DISULFID (1)..(7)
  MISC_FEATURE (1)..(12): /note=“Cylic peptide”
  MOD_RES (2)..(2): Thiazolidine-4-carboxylic acid
  MOD_RES (4)..(4): D-amino acid
  MOD_RES (8)..(8): Ornithine
  MOD_RES (9)..(9): Ornithine
CXHFHWCXXAAW

SEQ ID NO: 117 - protein, 12 aa, Artificial
  Synthetic
  DISULFID (1)..(7)
  MISC_FEATURE (1)..(12): /note=“Cyclic peptide”
  MOD_RES (2)..(2): Thiazolidine-4-carboxylic acid
  MOD_RES (4)..(4): D-amino acid
  MOD_RES (7)..(7): Penicillamine
CXHFHWXKKAAW

SEQ ID NO: 118 - protein, 9 aa, Artificial
  Synthetic
  DISULFID (1)..(7)
  MISC_FEATURE (1)..(9): /note=“Cyclic peptide”
  MOD_RES (2)..(2): Thiazolidine-4-carboxylic acid
  MOD_RES (4)..(4): D-amino acid
CXHFHWCKR

SEQ ID NO: 119 - protein, 12 aa, Artificial
  Synthetic
  DISULFID (1)..(7)
  MISC_FEATURE (1)..(12): /note=Cyclic peptide“
  MOD_RES (2)..(2): Thiazolidine-4-carboxylic acid
  MOD_RES (4)..(4): D-amino acid
  MOD_RES (9)..(9): Ornithine
  MOD_RES (11)..(11): Ornithine
CXHFHWCAXEXR

SEQ ID NO: 120 - protein, 12 aa, Artificial
  Synthetic
  DISULFID (1)..(7)
  MISC_FEATURE (1)..(12): /note=”Cyclic peptide“
  MOD_RES (2)..(2): Thiazolidine-4-carboxylic acid
  MOD_RES (4)..(4): D-amino acid
  MOD_RES (11)..(11): Ornithine
CXHFHWCAHEXR

SEQ ID NO: 121 - protein, 12 aa, Artificial
  Synthetic
  DISULFID (1)..(7)
  MISC_FEATURE (1)..(12): /note=”Cyclic peptide“
  MOD_RES (4)..(4): D-amino acid
  MOD_RES (10)..(10): D-amino acid
  MOD_RES (11)..(11): D-amino acid
CPHFHWCAHKKR